(19) United States
(12) Patent Application Publication
(10) Pub. No.: US 2026/0274807 A1
(43) Pub. Date: Sep. 17, 2026

(54) ERR MODULATORS

(71) Applicant: University of Health Sciences and Pharmacy in St. Louis, St. Louis, MO (US)

(72) Inventors: Bahaa ELGENDY, Ballwin, MO (US); Lamees Shamseldin HEGAZY, Ballwin, MO (US); Thomas Patrick BURRIS, Newberry, FL (US)

(21) Appl. No.: 19/653,509

(22) Filed: Apr. 21, 2026

Related U.S. Application Data
(63) Continuation of application No. PCT/US2024/053753, filed on Oct. 30, 2024.
(60) Provisional application No. 63/546,478, filed on Oct. 30, 2023.

(51) Int. Cl. C07D 271/06; A61K 31/4245; A61K 31/427; A61K 31/433; A61K 31/4439; A61K 31/4709; A61K 31/497; A61K 31/498; A61K 31/69; C07D 285/08; C07D 413/04; C07D 413/10; C07D 417/04; C07D 417/10; C07F 5/02

(52) U.S. Cl. CPC C07D 271/06; A61K 31/4245; A61K 31/427; A61K 31/433; A61K 31/4439; A61K 31/4709; A61K 31/497; A61K 31/498; A61K 31/69; C07D 285/08; C07D 413/04; C07D 413/10; C07D 417/04; C07D 417/10; C07F 5/027

(57) ABSTRACT

In one aspect, the present disclosure describes compounds which may be used to modulate the activity of an estrogen receptor-related orphan receptor (EER). Also described are pharmaceutical formulations, methods of synthesis and uses thereof.

### CROSS-REFERENCE TO RELATED APPLICATIONS

This application is a continuation of International Application No. PCT/US2024/053753, filed on Oct. 30, 2024, which claims the benefit of priority to U.S. Provisional Patent Application No. 63/546,478, filed Oct. 30, 2023, the entire contents of which are incorporated by reference herein.

This work was made with government support under grant number R21AG065657 by the National Institute on Aging of the National Institutes of Health. The government has certain rights in the invention.

### BACKGROUND

### 1. Field

This disclosure relates to the fields of medicine, pharmacology, chemistry, and metabolism. In particular, new compounds, compositions, and methods of treatment, relating to estrogen receptor-related orphan receptor are disclosed.

### 2. Related Art

The nuclear receptor (NR) superfamily constitutes a group of 48 transcription factors in humans, which includes the receptors for steroid hormones, thyroid hormone, lipophilic vitamins, and cholesterol metabolites (Manglesdorf et al., 1995; Evans, R. M., 1988). Approximately half of NRs are classified as orphan receptors since they do not have well characterized ligands (Kliewer et al., 1999; Giguere, V., 1999; Manglesdorf & Evans, 1995; Omalley & Conneely, 1992). Virtually all the NRs that have identified ligands are well-characterized targets for the development of drugs to treat myriad diseases including, diabetes, atherosclerosis, inflammation, and endocrine/reproductive disorders. NRs are proteins composed of multiple domains that provide a range of functions including DNA binding, small molecule ligand binding, and transcriptional regulatory activity (Manglesdorf et al., 1995; Evans, R. M., 1988). NRs function as transcription factors and typically regulate target gene transcription in a ligand-dependent manner. NRs recognize specific DNA response elements in the promoters/enhancers of their cognate target genes where they can respond to ligands by altering their ability to recruit a range of other transcriptional proteins that alter the rate of gene expression. Much about what is known about the mechanism of the ligand-dependent transcriptional regulation by NRs has been revealed by structure-function studies of the carboxy-terminial ligand binding domain (LBD). The LBD is a globular domain composed almost exclusively of α-helices arranged in a three layered “sandwich”. NR ligands bind to a ligand binding pocket (LBP) within the interior of this globular domain consistent with the typical hydrophobic character of NR ligands.

The estrogen receptor-related orphan receptors (ERRs) were the first orphan NRs to be identified. As their name indicates they are quite similar to the estrogen receptors (ERα and ERβ), but do not bind to endogenous ER ligands. Where the ERs function as obligate homodimers, the ERRs (ERRα, ERRβ, and ERRγ) function as monomers and bind to a DNA response element that are quite distinct from that of the ERs (Giguere, V., 2008). ERRα is widely expressed as is ERRγ, but greatest expression is noted in tissues with high energy demand such as the skeletal muscle, heart, adipose tissue, liver, and kidney (Giguere et al., 1988; Chen et al., 1999; Sladek et al., 1997). ERRβ is considerably more restricted in its pattern of expression where low levels of expression are noted in the liver, stomach, skeletal muscle, heart and kidney (Giguere et al., 1988; Chen et al., 1999). Unlike the ERs that require ligand binding to display transcriptional activity, all three ERRs display constitutive transcriptional activity in the absence of any ligand (Giguere, V., 2008). Given the lack of ligands that bind to these receptors, there remains a need to develop new compounds that bind to these ligands.

### SUMMARY

In one aspect, a method of modulating the activity of an estrogen related receptor comprises contacting the estrogen related receptor with a compound of the formula:

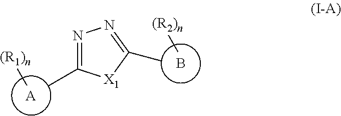

wherein:
X₁ is O or S; A is C₁₋₆ alkyl-aryl or C₆ aryl; B is C₁₋₆ alkyl, C₆₋₁₀ cycloalkyl,
C₁₋₆ alkyl-C₆₋₁₀ aryl, SC(O)-aryl, C₆₋₁₀ aryl, 5-10 membered heteroaryl, or N(H)C(O)-aryl; R₁ is H, halo, OH, N(R₃)₂, C₁₋₆ alkyl, C₁₋₆ haloalkyl, C₁₋₆ alkoxy, C(O)C₁₋₆ alkoxy, or B(OH)₂; R₂ is H, halo, N(R₃)₂, N(R₄)₂, C₁₋₆ alkyl, C₁₋₆ haloalkyl, C₁₋₆ alkoxy, C(O)C₁₋₆ alkoxy, C(O)OH, N(H)C(O)—C₃₋₆ cycloalkyl, or aryl; R₃ is H or O; R₄ is O or C₁₋₆ alkyl; m is 0, 1, or 2; and n is 0, 1, or 2; or a pharmaceutically acceptable salt thereof.

In some embodiments, the compound is further defined as:

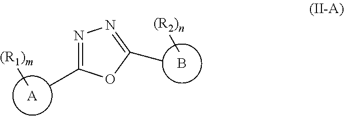

wherein A is C₁₋₆ alkyl-aryl or C₆ aryl; B is C₁₋₆ alkyl, C₁₋₆ alkyl-C₆₋₁₀ aryl, SC(O)-aryl, C₆₋₁₀ aryl, 5-10 membered heteroaryl, or N(H)C(O)-aryl; R₁ is H, halo, OH, N(R₃)₂, C₁₋₆ alkyl, C₁₋₆ haloalkyl, C₁₋₆ alkoxy, C(O)C₁₋₆ alkoxy, or B(OH)₂; R₂ is H, halo, N(R₄)₂, C₁₋₆ alkyl, C₁₋₆ haloalkyl, C₁₋₆ alkoxy, C(O)C₁₋₆ alkoxy, C(O)OH, or N(H)C(O)—C₃₋₆ cycloalkyl; R₃ is H or O; R₄ is 0 or C₁₋₆ alkyl; m is 0, 1, or 2; and n is 0 or 1; or a pharmaceutically acceptable salt thereof.

In some embodiments, the compound is further defined as:

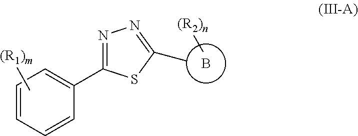

wherein B is C₁₋₆ alkyl-aryl, C₆₋₁₀ cycloalkyl, C₆₋₁₀ aryl, 5-10 membered heteroaryl, or N(H)C(O)-aryl; R₁ is H, halo, OH, N(R₃)₂, C₁₋₆ alkyl, C₁₋₆ haloalkyl, C₁₋₆ alkoxy, or B(OH)₂; R₂ is H, halo, N(R₃)₂, C₁₋₆ alkyl, C₁₋₆ haloalkyl, C₁₋₆ alkoxy, C(O)C₁₋₆ alkoxy, or aryl; R₃ is H or O; m is 0, 1, or 2; and n is 0, 1, or 2; or a pharmaceutically acceptable salt thereof.

In some embodiments, A is phenyl. In some embodiments, B is phenyl. In some embodiments, B is 5-10 membered heteroaryl. In some embodiments, R₁ is C₁₋₆ alkyl. In some embodiments, R₁ is C₁₋₆ haloalkyl. In some embodiments, R₁ is C₁₋₆ alkoxy. In some embodiments, R₁ is halo. In some embodiments, R₁ is B(OH)₂. In some embodiments, R₁ is selected from the group consisting of B(OH)₂, CF₃, CH₃, and OCH₃. In some embodiments, R₁ is B(OH)₂, Br, C(CH₃)₃, CF₃, CH₃, Cl, NH₂, OCH₃, OH, NO₂, or C(O)OCH₃. In some embodiments, R₂ is halo. In some embodiments, R₂ is N(R₃)₂. In some embodiments, R₂ is C₁₋₆ alkyl. In some embodiments, R₂ is C₁₋₆ haloalkyl. In some embodiments, R₂ is
C₁₋₆ alkoxy. In some embodiments, R₂ is C(CH₃)₃, C(O)OCH₃, C(O)OH, CF₃, CH₃, Cl, N(CH₃)₂, N(H)C(O)-cyclobutyl, N(H)C(O)-cyclohexyl, N(H)C(O)-cyclopropyl, NO₂, O(CH₂)₃CH₃, OCH₃, OH, Br, NH₂, or phenyl. In some embodiments, R₃ is O. In some embodiments, R₃ is H. In some embodiments, R₄ is O. In some embodiments, R₄ is C₁₋₆ alkyl.

In some embodiments, the compound is further defined as one selected from Table 1, Table 2, and pharmaceutically acceptable salts thereof.

In another aspect, the compound is in a pharmaceutical composition comprising a compound selected from Table 1, Table 2, and pharmaceutically acceptable salts thereof and one or more pharmaceutically acceptable carriers or vehicles.

In another aspect, the method results in modulation of the estrogen related receptor is sufficient to treat a disease or disorder. In some embodiments, the disease or disorder is associated with estrogen-related receptor α. In some embodiments, the disease or disorder is associated with estrogen-related receptor γ. In some embodiments, the disease or disorder is associated with cellular energy metabolism. In some embodiments, the disease or disorder is associated with glucose metabolism. In some embodiments, the disease or disorder is diabetes. In some embodiments, the disease or disorder is associated with lipid metabolism. In some embodiments, the disease or disorder is cancer. In some embodiments, the disease or disorder is breast cancer. In some embodiments, the disease or disorder is a bone disease or disorder. In some embodiments, the disease or disorder is associated with bone resorption.

In another aspect, a method of inhibiting the activity of an estrogen related receptor comprises administering to the subject of a compound selected from Table 1, Table 2, and pharmaceutically acceptable salts thereof.

In another aspect, a method of treating or lessening the severity of a disease or disorder in a subject comprises administering to the subject a compound selected from Table 1, Table 2, and pharmaceutically acceptable salts thereof. In some embodiments, the disease or disorder is selected from the group consisting of diabetes, breast cancer, bone disease, bone resorption, heart failure, obesity, metabolic disease, muscle wasting and other diseases of muscle function, diseases of mitochondrial disfunction, kidney diseases and disfunction, and neurodegenerative diseases including AD, PD, and ALS.

As provided herein, the present disclosure relates to compounds that modulate the activity of estrogen receptor-related orphan receptors. In some embodiments, the present disclosure provides compounds of the formula:

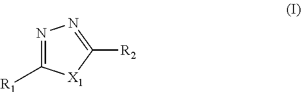

wherein:
   X₁ is O or S wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some aspects, the present disclosure provides methods of modulating the activity of an estrogen related receptor comprising contacting the estrogen related receptor with a compound of the formula:

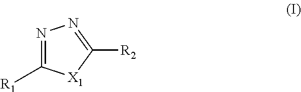

wherein:
   X₁ is O or S, wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some embodiments, the compound is further defined as:

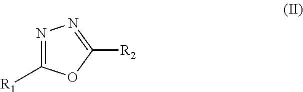

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some embodiments, the compound is further defined as:

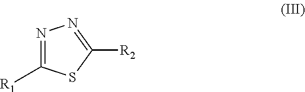

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some embodiments, X₁ is O. In other embodiments, X₁ is S.

In some embodiments, R₁ and R₂ are the same. In other embodiments, R₁ and R₂ are different.

In some embodiments, R₁ is cycloalkyl_{(C≤12)} or substituted cycloalkyl_{(C≤12)}. In some embodiments, R₁ is cycloalkyl_{(C≤12)} such as cyclohexyl or adamantyl. In other embodiments, R₁ is aralkyl_{(C≤12)} or substituted aralkyl_{(C≤12)}. In some embodiments, R₁ is substituted aralkyl_{(C≤12)} such as 2-nitrobenzyl. In other embodiments, R₁ is aryl_{(C≤12)} or substituted aryl_{(C≤12)}. In some embodiments, R₁ is aryl_{(C≤12)} such as phenyl or 4-methylphenyl. In other embodiments, R₁ is substituted aryl_{(C≤12)} such as 4-bromophenyl, 4-chlorophenyl, 2-nitrophenyl, 3-nitrophenyl, 4-nitrophenyl, 4-methoxyphenyl, 3-trifluoromethylphenyl, 4-trifluoromethylphenyl, 2-aminophenyl, 3-aminophenyl, 4-aminophenyl, 2-amino-4-methoxyphenyl, 2-amino-4-bromophenyl, 2-amino-5-bromophenyl, 2-amino-5-methoxyphenyl, 2-amino-3-methoxyphenyl, 2-amino-3-chlorophenyl, 2-nitro-5-bromophenyl, 2-nitro-4-bromophenyl, 2-nitro-4-methoxyphenyl, 2-nitro-5-methoxyphenyl, 2-nitro-3-methoxyphenyl, or 2-nitro-3-chlorophenyl.

In some embodiments, R₂ is alkyl_{(C≤12)} or substituted alkyl_{(C≤12)}. In some embodiments, R₂ is alkyl_{(C≤12)} such as ethyl. In other embodiments, R₂ is aryl_{(C≤12)} or substituted aryl_{(C≤12)}. In some embodiments, R₂ is aryl_{(C≤12)} such as phenyl, 2-methylphenyl, 4-methylphenyl, or 4-t-butylphenyl. In other embodiments, R₂ is substituted aryl_{(C≤12)} such as 2-nitrophenyl, 2-chlorophenyl, 4-chlorophenyl, 4-methoxyphenyl, 3-methoxyphenyl, 3-nitrophenyl, 4-dimethylaminophenyl, 4-trifluorophenyl, 4-bromophenyl, or 4-chlorophenyl. In other embodiments, R₂ is heteroaryl_{(C≤12)} or substituted heteroaryl_{(C≤12)}. In some embodiments, R₂ is heteroaryl_{(C≤12)} such as 2-pyrindinyl, 2-quinolinyl, 4-quinolinyl, furanyl, thiophenyl, or 5-methylfuranyl.

In some embodiments, the methods are performed in vivo. In other embodiments, the methods are performed in vitro. In other embodiments, the methods are performed ex vivo. In some embodiments, the methods result in modulation of the estrogen related receptor is sufficient to treat a disease or disorder. In some embodiments, the disease or disorder is associated with estrogen-related receptor α. In some embodiments, the disease or disorder is associated with estrogen-related receptor β. In other embodiments, the disease or disorder is associated with estrogen-related receptor γ. In some embodiments, the disease or disorder is associated with cellular energy metabolism. In some embodiments, the disease or disorder is associated with glucose metabolism such as diabetes. In other embodiments, the disease or disorder is associated with lipid metabolism. In other embodiments, the disease or disorder is cancer such as breast cancer. In other embodiments, the disease or disorder is a bone disease or disorder. In some embodiments, the disease or disorder is associated with bone resorption.

In yet another aspect, the present disclosure provides methods of treating a disease or disorder in a patient in need thereof comprising administering to the patient a therapeutically effective amount of a compound of the formula:

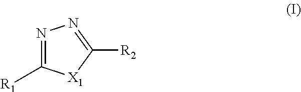

wherein:
   X₁ is O or S, wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some embodiments, the compound is further defined as:

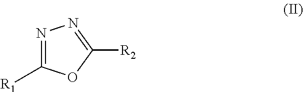

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some embodiments, the compound is further defined as:

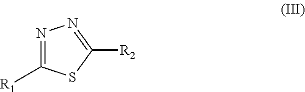

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

In some embodiments, X₁ is O. In other embodiments, X₁ is S. In still other embodiments, X₁ is NRₐ.

In some embodiments, R₁ and R₂ are the same. In other embodiments, R₁ and R₂ are different.

In some embodiments, R₁ is cycloalkyl_{(C≤12)} or substituted cycloalkyl_{(C≤12)}. In some embodiments, R₁ is cycloalkyl_{(C≤12)} such as cyclohexyl or adamantyl. In other embodiments, R₁ is aralkyl_{(C≤12)} or substituted aralkyl_{(C≤12)}. In some embodiments, R₁ is substituted aralkyl_{(C≤12)} such as 2-nitrobenzyl. In other embodiments, R₁ is aryl_{(C≤12)} or substituted aryl_{(C≤12)}. In some embodiments, R₁ is aryl_{(C≤12)} such as phenyl or 4-methylphenyl. In other embodiments, R₁ is substituted aryl_{(C≤12)} such as 4-bromophenyl, 4-chlorophenyl, 2-nitrophenyl, 3-nitrophenyl, 4-nitrophenyl, 4-methoxyphenyl, 3-trifluoromethylphenyl, 4-trifluoromethylphenyl, 2-aminophenyl, 3-aminophenyl, 4-aminophenyl, 2-amino-4-methoxyphenyl, 2-amino-4-bromophenyl, 2-amino-5-bromophenyl, 2-amino-5-methoxyphenyl, 2-amino-3-methoxyphenyl, 2-amino-3-chlorophenyl, 2-nitro-5-bromophenyl, 2-nitro-4-bromophenyl, 2-nitro-4-methoxyphenyl, 2-nitro-5-methoxyphenyl, 2-nitro-3-methoxyphenyl, or 2-nitro-3-chlorophenyl.

In some embodiments, R₂ is alkyl_{(C≤12)} or substituted alkyl_{(C≤12)}. In some embodiments, R₂ is alkyl_{(C≤12)} such as ethyl. In other embodiments, R₂ is aryl_{(C≤12)} or substituted aryl_{(C≤12)}. In some embodiments, R₂ is aryl_{(C≤12)} such as phenyl, 2-methylphenyl, 4-methylphenyl, or 4-t-butylphenyl. In other embodiments, R₂ is substituted aryl_{(C≤12)} such as 2-nitrophenyl, 2-chlorophenyl, 4-chlorophenyl, 4-methoxyphenyl, 3-methoxyphenyl, 3-nitrophenyl, 4-dimethylaminophenyl, 4-trifluorophenyl, 4-bromophenyl, or 4-chlorophenyl. In other embodiments, R₂ is heteroaryl_{(C≤12)} or substituted heteroaryl_{(C≤12)}. In some embodiments, R₂ is heteroaryl_{(C≤12)} such as 2-pyrindinyl, 2-quinolinyl, 4-quinolinyl, furanyl, thiophenyl, or 5-methylfuranyl.

In some embodiments, the disease or disorder is associated with estrogen-related receptor α. In other embodiments, the disease or disorder is associated with estrogen-related receptor β. In other embodiments, the disease or disorder is associated with estrogen-related receptor γ. In some embodiments, the disease or disorder is associated with cellular energy metabolism. In some embodiments, the disease or disorder is associated with glucose metabolism such as diabetes. In other embodiments, the disease or disorder is associated with lipid metabolism. In other embodiments, the disease or disorder is cancer such as breast cancer. In other embodiments, the disease or disorder is a bone disease or disorder. In some embodiments, the disease or disorder is associated with bone resorption.

In some embodiments, the methods further comprise administering a second therapeutic agent. In some embodiments, the compound is formulated in a pharmaceutical composition. In some embodiments, the pharmaceutical composition further comprises an excipient. In some embodiments, the pharmaceutical composition is formulated for administration orally, topically, intravenously, or via injection. In some embodiments, the pharmaceutical composition is formulated as a unit dose. In some embodiments, the patient is a mammal. In some embodiments, the mammal is a human. In some embodiments, the compound is administered once. In other embodiments, the compound is administered two or more times. In some embodiments, the compound is administered daily, weekly, or monthly.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### EXAMPLES

Provided herein are synthetic ligands that modulate the activity of ERRs. Some of these compounds have been shown to be active against ERRα and ERRγ. These compounds may be used to show a micromolar or less activity in the one or more of ERR. These compounds may be used to treat one or more conditions for which modulation of the activity of one or more ERR may be useful. Some of these conditions include bone diseases or disorders, cancers, or metabolic disorders such as diabetes, obesity, lipid metabolism, or muscle wasting disorders. Furthermore, these compounds may represent an improvement over those known in the art as the compounds may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties. These and more details will be discussed in more detail below.

### I. COMPOUNDS AND FORMULATIONS THEREOF

### A. Compounds

The compounds of the present disclosure may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

In yet another aspect, the present disclosure provides methods of treating a disease or disorder in a patient in need thereof comprising administering to the patient a therapeutically effective amount of a compound of the formula:

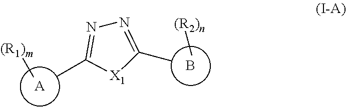

wherein:
   X₁ is O or S;
   A is C₁₋₆ alkyl-aryl or C₆ aryl;
   B is C₁₋₆ alkyl, C₆₋₁₀ cycloalkyl, C₁₋₆ alkyl-C₆₋₁₀ aryl, SC(O)-aryl, C₆₋₁₀ aryl,
   5-10 membered heteroaryl, or N(H)C(O)-aryl;
   R₁ is H, halo, OH, N(R₃)₂, C₁₋₆ alkyl, C₁₋₆ haloalkyl, C₁₋₆ alkoxy, C(O)C₁₋₆ alkoxy, or B(OH)₂;
   R₂ is H, halo, N(R₃)₂, N(R₄)₂, C₁₋₆ alkyl, C₁₋₆ haloalkyl, C₁₋₆ alkoxy, C(O)C₁₋₆ alkoxy, C(O)OH, N(H)C(O)—C₃₋₆ cycloalkyl, or aryl;
   R₃ is H or O;
   R₄ is O or C₁₋₆ alkyl;
   m is 0, 1, or 2; and
   n is 0, 1, or 2;
or a pharmaceutically acceptable salt thereof.

In some embodiments, the disclosure relates to a compound of formula (I-A), wherein X₁ is selected from the group consisting of O or S. In some embodiments, X₁ is O. In some embodiments, X₁ is S.

In some embodiments, the disclosure relates to a compound of formula (I-A), wherein A is selected from the group consisting of C₁₋₆ alkyl-aryl or C₆ aryl. In some embodiments, A is C₁₋₆ alkyl-aryl. In some embodiments, A is C₆ aryl. In some embodiments, A is CH₂-phenyl. In some embodiments, A is phenyl.

In some embodiments, the disclosure relates to a compound of formula (I-A), wherein B is selected from the group consisting of C₁₋₆ alkyl, C₆₋₁₀ cycloalkyl, C₁₋₆ alkyl-C₆₋₁₀ aryl, SC(O)-aryl, C₆₋₁₀ aryl, 5-10 membered heteroaryl, or N(H)C(O)-aryl. In some embodiments, B is C₁₋₆ alkyl. In some embodiments, B is C₆₋₁₀ cycloalkyl. In some embodiments, B is C₁₋₆ alkyl-C₆₋₁₀ aryl. In some embodiments, B is SC(O)-aryl. In some embodiments, B is C₆₋₁₀ aryl. In some embodiments, B is 5-10 membered heteroaryl. In some embodiments, B is N(H)C(O)-aryl. In some embodiments, B is CH₂CH₃. In some embodiments, B is CH₂-napthyl. In some embodiments, B is CH₂-phenyl. In some embodiments, B is furan. In some embodiments, B is N(H)C(O)-phenyl. In some embodiments, B is napthyl. In some embodiments, B is phenyl. In some embodiments, B is pyrazole. In some embodiments, B is pyridine. In some embodiments, B is quinoline. In some embodiments, B is SC(O)-phenyl. In some embodiments, B is thiazole. In some embodiments, B is thiophene. In some embodiments, B is adamantane. In some embodiments, B is cyclohexyl. In some embodiments, B is imidazole. In some embodiments, B is oxazole. In some embodiments, B is pyrazine. In some embodiments, B is pyrimidine. In some embodiments, B is quinoxaline.

In some embodiments, the disclosure relates to a compound of formula (I-A), wherein R₁ is selected from the group consisting of H, halo, OH, N(R₃)₂, C₁₋₆ alkyl, C₁₋₆ haloalkyl, C₁₋₆ alkoxy, C(O)C₁₋₆ alkoxy, or B(OH)₂. In some embodiments, R₁ is H. In some embodiments, R₁ is halo. In some embodiments, R₁ is OH. In some embodiments, R₁ is N(R₃)₂. In some embodiments, R₁ is C₁₋₆ alkyl. In some embodiments, R₁ is C₁₋₆ haloalkyl. In some embodiments, R₁ is C₁₋₆ alkoxy. In some embodiments, R₁ is C(O)C₁₋₆ alkoxy. In some embodiments, R₁ is B(OH)₂. In some embodiments, R₁ is Br. In some embodiments, R₁ is C(CH₃)₃. In some embodiments, R₁ is CF₃. In some embodiments, R₁ is CH₃. In some embodiments, R₁ is Cl. In some embodiments, R₁ is NH₂. In some embodiments, R₁ is OCH₃. In some embodiments, R₁ is OH. In some embodiments, R₁ is NO₂. In some embodiments, R₁ is C(O)OCH₃.

In some embodiments, the disclosure relates to a compound of formula (I-A), wherein R₂ is selected from the group consisting of H, halo, N(R₃)₂, N(R₄)₂, C₁₋₆ alkyl, C₁₋₆ haloalkyl, C₁₋₆ alkoxy, C(O)C₁₋₆ alkoxy, C(O)OH, N(H)C(O)—C₃₋₆ cycloalkyl, or aryl. In some embodiments, R₂ is H. In some embodiments, R₂ is halo. In some embodiments, R₂ is N(R₃)₂. In some embodiments, R₂ is N(R₄)₂. In some embodiments, R₂ is C₁₋₆ alkyl. In some embodiments, R₂ is C₁₋₆ haloalkyl. In some embodiments, R₂ is C₁₋₆ alkoxy. In some embodiments, R₂ is C(O)C₁₋₆ alkoxy. In some embodiments, R₂ is C(O)OH. In some embodiments, R₂ is N(H)C(O)—C₃₋₆ cycloalkyl. In some embodiments, R₂ is aryl. In some embodiments, R₂ is C(CH₃)₃. In some embodiments, R₂ is C(O)OCH₃. In some embodiments, R₂ is C(O)OH. In some embodiments, R₂ is CF₃. In some embodiments, R₂ is CH₃. In some embodiments, R₂ is Cl. In some embodiments, R₂ is N(CH₃)₂. In some embodiments, R₂ is N(H)C(O)-cyclobutyl. In some embodiments, R₂ is N(H)C(O)-cyclohexyl. In some embodiments, R₂ is N(H)C(O)-cyclopropyl. In some embodiments, R₂ is NO₂. In some embodiments, R₂ is O(CH₂)₃CH₃. In some embodiments, R₂ is OCH₃. In some embodiments, R₂ is OH. In some embodiments, R₂ is Br. In some embodiments, R₂ is NH₂. In some embodiments, R₂ is phenyl.

In some embodiments, the disclosure relates to a compound of formula (I-A), wherein R₃ is selected from the group consisting of H or O. In some embodiments, R₃ is H. In some embodiments, R₃ is O.

In some embodiments, the disclosure relates to a compound of formula (I-A), wherein R₄ is selected from the group consisting of O or C₁₋₆ alkyl. In some embodiments, R₄ is C₁₋₆ alkyl. In some embodiments, R₄ is O.

In some embodiments, the disclosure relates to a compound of formula (I-A), wherein n is selected from the group consisting of 0, 1, or 2. In some embodiments, n is 0. In some embodiments, n is 1. In some embodiments, n is 2.

In some embodiments, the disclosure relates to a compound of formula (I-A), wherein m is selected from the group consisting of 0, 1, or 2. In some embodiments, m is 0. In some embodiments, m is 1. In some embodiments, m is 2.

In some embodiments, the compound is further defined as:

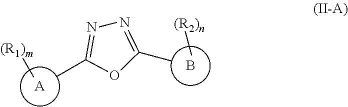

wherein:
   A is C₁₋₆ alkyl-aryl or C₆ aryl;
   B is C₁₋₆ alkyl, C₁₋₆ alkyl-C₆₋₁₀ aryl, SC(O)-aryl, C₆₋₁₀ aryl, 5-10 membered heteroaryl, or N(H)C(O)-aryl;
   R₁ is H, halo, OH, N(R₃)₂, C₁₋₆ alkyl, C₁₋₆ haloalkyl, C₁₋₆ alkoxy, C(O)C₁₋₆ alkoxy, or B(OH)₂;
   R₂ is H, halo, N(R₄)₂, C₁₋₆ alkyl, C₁₋₆ haloalkyl, C₁₋₆ alkoxy, C(O)C₁₋₆ alkoxy, C(O)OH, or N(H)C(O)—C₃₋₆ cycloalkyl;
   R₃ is H or O;
   R₄ is O or C₁₋₆ alkyl;
   m is 0, 1, or 2; and
   n is 0 or 1;
or a pharmaceutically acceptable salt thereof.

In some embodiments, the disclosure relates to a compound of formula (II-A), wherein A is selected from the group consisting of C₁₋₆ alkyl-aryl or C₆ aryl. In some embodiments, A is C₁₋₆ alkyl-aryl. In some embodiments, A is C₆ aryl. In some embodiments, A is CH₂-phenyl. In some embodiments, A is phenyl.

In some embodiments, the disclosure relates to a compound of formula (II-A), wherein B is selected from the group consisting of C₁₋₆ alkyl, C₁₋₆ alkyl-C₆₋₁₀ aryl, SC(O)-aryl, C₆₋₁₀ aryl, 5-10 membered heteroaryl, or N(H)C(O)-aryl. In some embodiments, B is C₁₋₆ alkyl. In some embodiments, B is C₁₋₆ alkyl-C₆₋₁₀ aryl. In some embodiments, B is SC(O)-aryl. In some embodiments, B is C₆₋₁₀ aryl. In some embodiments, B is 5-10 membered heteroaryl. In some embodiments, B is N(H)C(O)-aryl. In some embodiments, B is CH₂CH₃. In some embodiments, B is CH₂-napthyl. In some embodiments, B is furan. In some embodiments, B is N(H)C(O)-phenyl. In some embodiments, B is napthyl. In some embodiments, B is phenyl. In some embodiments, B is pyrazole. In some embodiments, B is pyridine. In some embodiments, B is quinoline. In some embodiments, B is SC(O)-phenyl. In some embodiments, B is thiazole. In some embodiments, B is thiophene.

In some embodiments, the disclosure relates to a compound of formula (II-A), wherein R₁ is selected from the group consisting of H, halo, OH, N(R₃)₂, C₁₋₆ alkyl, C₁₋₆ haloalkyl, C₁₋₆ alkoxy, C(O)C₁₋₆ alkoxy, or B(OH)₂. In some embodiments, R₁ is H. In some embodiments, R₁ is halo. In some embodiments, R₁ is OH. In some embodiments, R₁ is N(R₃)₂. In some embodiments, R₁ is C₁₋₆ alkyl. In some embodiments, R₁ is C₁₋₆ haloalkyl. In some embodiments, R₁ is C₁₋₆ alkoxy. In some embodiments, R₁ is C(O)C₁₋₆ alkoxy. In some embodiments, R₁ is B(OH)₂. In some embodiments, R₁ is Br. In some embodiments, R₁ is CF₃. In some embodiments, R₁ is CH₃. In some embodiments, R₁ is Cl. In some embodiments, R₁ is NH₂. In some embodiments, R₁ is OCH₃. In some embodiments, R₁ is OH. In some embodiments, R₁ is NO₂. In some embodiments, R₁ is C(O)OCH₃.

In some embodiments, the disclosure relates to a compound of formula (II-A), wherein R₂ is selected from the group consisting of H, halo, N(R₃)₂, N(R₄)₂, C₁₋₆ alkyl, C₁₋₆ haloalkyl, C₁₋₆ alkoxy, C(O)C₁₋₆ alkoxy, C(O)OH, or N(H)C(O)—C₃₋₆ cycloalkyl. In some embodiments, R₂ is H. In some embodiments, R₂ is halo. In some embodiments, R₂ is N(R₃)₂. In some embodiments, R₂ is N(R₄)₂. In some embodiments, R₂ is C₁₋₆ alkyl. In some embodiments, R₂ is C₁₋₆ haloalkyl. In some embodiments, R₂ is C₁₋₆ alkoxy. In some embodiments, R₂ is C(O)C₁₋₆ alkoxy. In some embodiments, R₂ is C(O)OH. In some embodiments, R₂ is N(H)C(O)—C₃₋₆ cycloalkyl. In some embodiments, R₂ is C(CH₃)₃. In some embodiments, R₂ is C(O)OCH₃. In some embodiments, R₂ is C(O)OH. In some embodiments, R₂ is CF₃. In some embodiments, R₂ is CH₃. In some embodiments, R₂ is Cl. In some embodiments, R₂ is N(CH₃)₂. In some embodiments, R₂ is N(H)C(O)-cyclobutyl. In some embodiments, R₂ is N(H)C(O)-cyclohexyl. In some embodiments, R₂ is
N(H)C(O)-cyclopropyl. In some embodiments, R₂ is NO₂. In some embodiments, R₂ is O(CH₂)₃CH₃. In some embodiments, R₂ is OCH₃. In some embodiments, R₂ is OH.

In some embodiments, the disclosure relates to a compound of formula (II-A), wherein R₃ is selected from the group consisting of H or O. In some embodiments, R₃ is H. In some embodiments, R₃ is O.

In some embodiments, the disclosure relates to a compound of formula (II-A), wherein R₄ is selected from the group consisting of O or C₁₋₆ alkyl. In some embodiments, R₄ is C₁₋₆ alkyl. In some embodiments, R₄ is O.

In some embodiments, the disclosure relates to a compound of formula (II-A), wherein n is selected from the group consisting of 0, 1, or 2. In some embodiments, n is 0. In some embodiments, n is 1. In some embodiments, n is 2.

In some embodiments, the disclosure relates to a compound of formula (II-A), wherein m is selected from the group consisting of 0 or 1. In some embodiments, m is 0. In some embodiments, m is 1.

In some embodiments, the compound is further defined as:

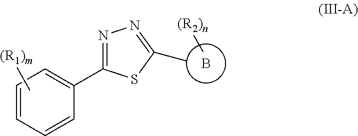

wherein:
   B is C₁₋₆ alkyl-aryl, C₆₋₁₀ cycloalkyl, C₆₋₁₀ aryl, 5-10 membered heteroaryl, or N(H)C(O)-aryl;
   R₁ is H, halo, OH, N(R₃)₂, C₁₋₆ alkyl, C₁₋₆ haloalkyl, C₁₋₆ alkoxy, or B(OH)₂;
   R₂ is H, halo, N(R₃)₂, C₁₋₆ alkyl, C₁₋₆ haloalkyl, C₁₋₆ alkoxy, C(O)C₁₋₆ alkoxy, or aryl;
   R₃ is H or O;
   m is 0, 1, or 2; and
   n is 0, 1, or 2;
or a pharmaceutically acceptable salt thereof.

In some embodiments, the disclosure relates to a compound of formula (III-A), wherein B is selected from the group consisting of C₆₋₁₀ cycloalkyl, C₁₋₆ alkyl-C₆₋₁₀ aryl, C₆₋₁₀ aryl, 5-10 membered heteroaryl, or N(H)C(O)-aryl. In some embodiments, B is C₆₋₁₀ cycloalkyl. In some embodiments, B is C₁₋₆ alkyl-C₆₋₁₀ aryl. In some embodiments, B is C₆₋₁₀ aryl. In some embodiments, B is 5-10 membered heteroaryl. In some embodiments, B is N(H)C(O)-aryl. In some embodiments, B is furan. In some embodiments, B is N(H)C(O)-phenyl. In some embodiments, B is napthyl. In some embodiments, B is phenyl. In some embodiments, B is pyrazole. In some embodiments, B is pyridine. In some embodiments, B is quinoline. In some embodiments, B is thiazole. In some embodiments, B is thiophene. In some embodiments, B is adamantane. In some embodiments, B is cyclohexyl. In some embodiments, B is imidazole. In some embodiments, B is oxazole. In some embodiments, B is pyrazine. In some embodiments, B is pyrimidine. In some embodiments, B is quinoxaline. In some embodiments, B is CH₂-phenyl.

In some embodiments, the disclosure relates to a compound of formula (III-A), wherein R₁ is selected from the group consisting of H, halo, OH, N(R₃)₂, C₁₋₆ alkyl, C₁₋₆ haloalkyl, C₁₋₆ alkoxy, or B(OH)₂. In some embodiments, R₁ is H. In some embodiments, R₁ is halo. In some embodiments, R₁ is OH. In some embodiments, R₁ is N(R₃)₂. In some embodiments, R₁ is C₁₋₆ alkyl. In some embodiments, R₁ is C₁₋₆ haloalkyl. In some embodiments, R₁ is C₁₋₆ alkoxy. In some embodiments, R₁ is B(OH)₂. In some embodiments, R₁ is Br. In some embodiments, R₁ is C(CH₃)₃. In some embodiments, R₁ is CF₃. In some embodiments, R₁ is CH₃. In some embodiments, R₁ is Cl. In some embodiments, R₁ is NH₂. In some embodiments, R₁ is OCH₃. In some embodiments, R₁ is OH. In some embodiments, R₁ is NO₂.

In some embodiments, the disclosure relates to a compound of formula (III-A), wherein R₂ is selected from the group consisting of H, halo, N(R₃)₂, C₁₋₆ alkyl, C₁₋₆ haloalkyl, C₁₋₆ alkoxy, C(O)C₁₋₆ alkoxy, or aryl. In some embodiments, R₂ is H. In some embodiments, R₂ is halo. In some embodiments, R₂ is N(R₃)₂. In some embodiments, R₂ is C₁₋₆ alkyl. In some embodiments, R₂ is C₁₋₆ haloalkyl. In some embodiments, R₂ is C₁₋₆ alkoxy. In some embodiments, R₂ is C(O)C₁₋₆ alkoxy. In some embodiments, R₂ is aryl. In some embodiments, R₂ is C(CH₃)₃. In some embodiments, R₂ is C(O)OCH₃. In some embodiments, R₂ is CF₃. In some embodiments, R₂ is CH₃. In some embodiments, R₂ is Cl. In some embodiments, R₂ is NO₂. In some embodiments, R₂ is O(CH₂)₃CH₃. In some embodiments, R₂ is OCH₃. In some embodiments, R₂ is OH. In some embodiments, R₂ is Br. In some embodiments, R₂ is NH₂. In some embodiments, R₂ is phenyl.

In some embodiments, the disclosure relates to a compound of formula (III-A), wherein R₃ is selected from the group consisting of H or O. In some embodiments, R₃ is H. In some embodiments, R₃ is O.

In some embodiments, the disclosure relates to a compound of formula (III-A), wherein n is selected from the group consisting of 0, 1, or 2. In some embodiments, n is 0. In some embodiments, n is 1. In some embodiments, n is 2.

In some embodiments, the disclosure relates to a compound of formula (III-A), wherein m is selected from the group consisting of 0, 1, or 2. In some embodiments, m is 0. In some embodiments, m is 1. In some embodiments, m is 2.

In some embodiments, R₁ and R₂ are the same. In other embodiments, R₁ and R₂ are different.

TABLE 1
Series A Compounds

| Entry | Structure |
|---|---|
| 1 | 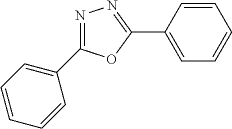 [SMILES: c1ccc(-c2nnc(-c3ccccc3)o2)cc1] |
| 2 | 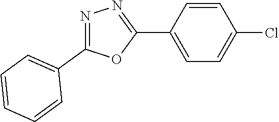 [SMILES: Clc1ccc(-c2nnc(-c3ccccc3)o2)cc1] |
| 3 | 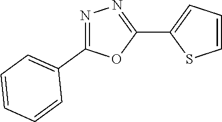 [SMILES: c1ccc(-c2nnc(-c3cccs3)o2)cc1] |
| 4 | 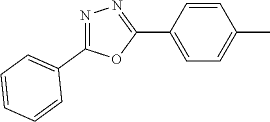 [SMILES: Cc1ccc(-c2nnc(-c3ccccc3)o2)cc1] |
| 5 | 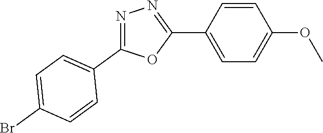 [SMILES: COc1ccc(-c2nnc(-c3ccc(Br)cc3)o2)cc1] |
| 6 | 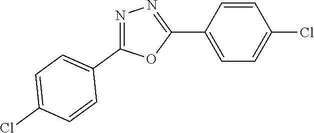 [SMILES: Clc1ccc(-c2nnc(-c3ccc(Cl)cc3)o2)cc1] |
| 7 | 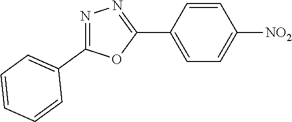 [SMILES: O=[N+]([O-])c1ccc(-c2nnc(-c3ccccc3)o2)cc1] |
| 8 | 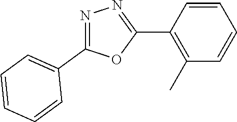 [SMILES: Cc1ccccc1-c1nnc(-c2ccccc2)o1] |
| 9 | 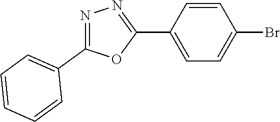 [SMILES: Brc1ccc(-c2nnc(-c3ccccc3)o2)cc1] |
| 10 | 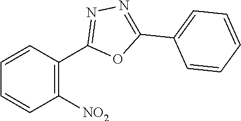 [SMILES: O=[N+]([O-])c1ccccc1-c1nnc(-c2ccccc2)o1] |
| 11 | 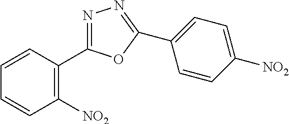 [SMILES: O=[N+]([O-])c1ccc(-c2nnc(-c3ccccc3[N+](=O)[O-])o2)cc1] |
| 12 | 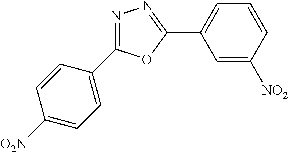 [SMILES: O=[N+]([O-])c1ccc(-c2nnc(-c3cccc([N+](=O)[O-])c3)o2)cc1] |
| 13 | 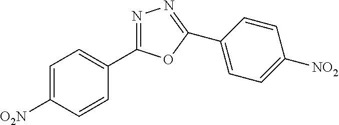 [SMILES: O=[N+]([O-])c1ccc(-c2nnc(-c3ccc([N+](=O)[O-])cc3)o2)cc1] |
| 14 | 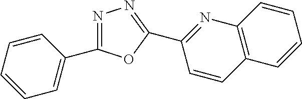 [SMILES: c1ccc(-c2nnc(-c3ccc4ccccc4n3)o2)cc1] |
| 15 | 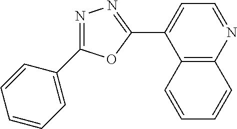 [SMILES: c1ccc(-c2nnc(-c3ccnc4ccccc34)o2)cc1] |
| 16 | 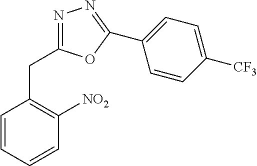 [SMILES: O=[N+]([O-])c1ccccc1Cc1nnc(-c2ccc(C(F)(F)F)cc2)o1] |
| 17 | 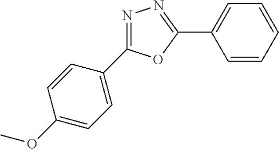 [SMILES: COc1ccc(-c2nnc(-c3ccccc3)o2)cc1] |
| 18 | 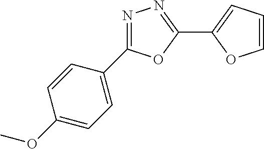 [SMILES: COc1ccc(-c2nnc(-c3ccco3)o2)cc1] |
| 19 | 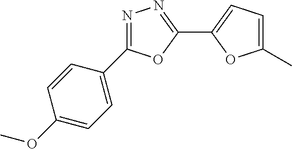 |
| 20 | 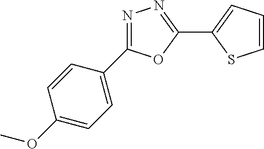 [SMILES: COc1ccc(-c2nnc(-c3cccs3)o2)cc1] |
| 21 | 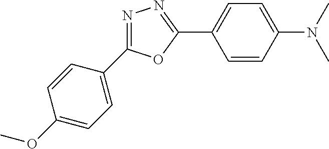 |
| 22 | 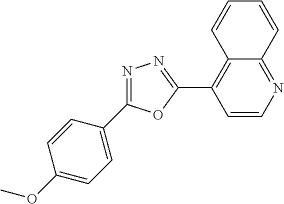 [SMILES: COc1ccc(-c2nnc(-c3ccnc4ccccc34)o2)cc1] |
| 23 | 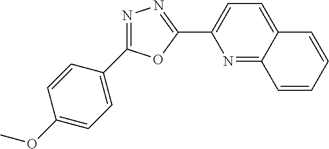 [SMILES: COc1ccc(-c2nnc(-c3ccc4ccccc4n3)o2)cc1] |
| 24 | 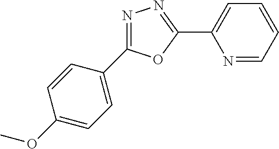 [SMILES: COc1ccc(-c2nnc(-c3ccccn3)o2)cc1] |
| 25 | 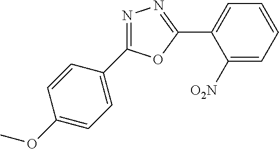 [SMILES: COc1ccc(-c2nnc(-c3ccccc3[N+](=O)[O-])o2)cc1] |
| 26 | 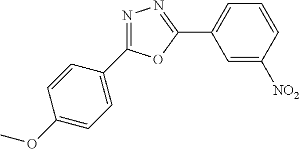 [SMILES: COc1ccc(-c2nnc(-c3cccc([N+](=O)[O-])c3)o2)cc1] |
| 27 | 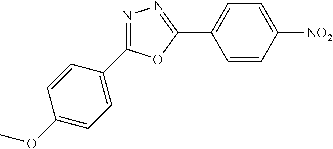 [SMILES: COc1ccc(-c2nnc(-c3ccc([N+](=O)[O-])cc3)o2)cc1] |
| 28 | 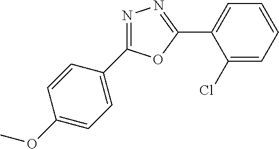 [SMILES: COc1ccc(-c2nnc(-c3ccccc3Cl)o2)cc1] |
| 29 | 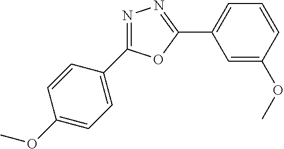 [SMILES: COc1ccc(-c2nnc(-c3cccc(OC)c3)o2)cc1] |
| 30 | 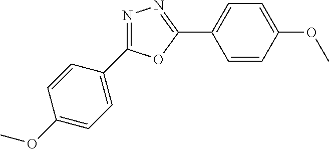 |
| 31 | 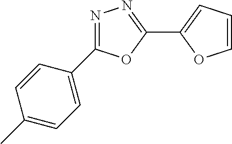 [SMILES: Cc1ccc(-c2nnc(-c3ccco3)o2)cc1] |
| 32 | 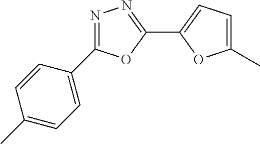 |
| 33 | 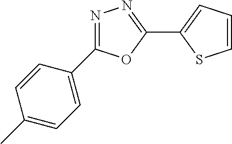 [SMILES: Cc1ccc(-c2nnc(-c3cccs3)o2)cc1] |
| 34 | 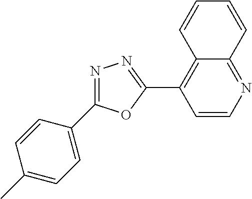 [SMILES: Cc1ccc(-c2nnc(-c3ccnc4ccccc34)o2)cc1] |
| 35 | 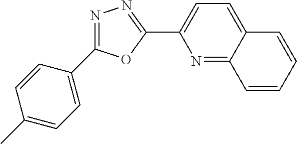 [SMILES: Cc1ccc(-c2nnc(-c3ccc4ccccc4n3)o2)cc1] |
| 36 | 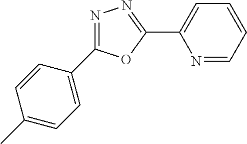 [SMILES: Cc1ccc(-c2nnc(-c3ccccn3)o2)cc1] |
| 37 | 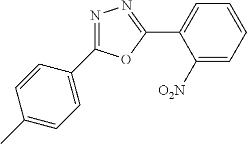 [SMILES: Cc1ccc(-c2nnc(-c3ccccc3[N+](=O)[O-])o2)cc1] |
| 38 | 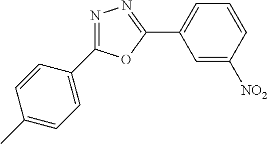 [SMILES: Cc1ccc(-c2nnc(-c3cccc([N+](=O)[O-])c3)o2)cc1] |
| 39 | 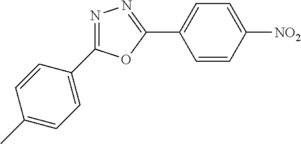 [SMILES: Cc1ccc(-c2nnc(-c3ccc([N+](=O)[O-])cc3)o2)cc1] |
| 40 | 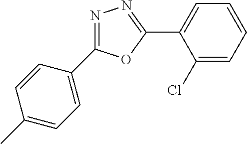 [SMILES: Cc1ccc(-c2nnc(-c3ccccc3Cl)o2)cc1] |
| 41 | 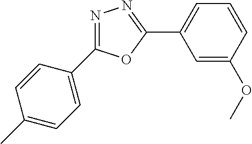 [SMILES: COc1cccc(-c2nnc(-c3ccc(C)cc3)o2)c1] |
| 42 | 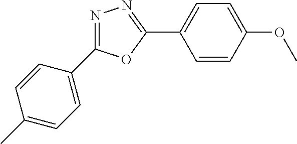 |
| 43 | 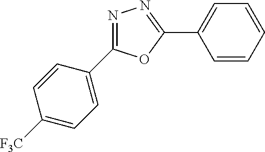 [SMILES: FC(F)(F)c1ccc(-c2nnc(-c3ccccc3)o2)cc1] |
| 44 | 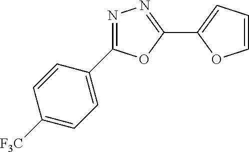 [SMILES: FC(F)(F)c1ccc(-c2nnc(-c3ccco3)o2)cc1] |
| 45 | 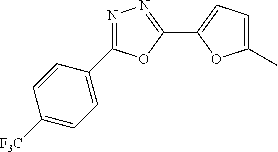 [SMILES: Cc1ccc(-c2nnc(-c3ccc(C(F)(F)F)cc3)o2)o1] |
| 46 | 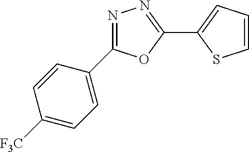 [SMILES: FC(F)(F)c1ccc(-c2nnc(-c3cccs3)o2)cc1] |
| 47 | 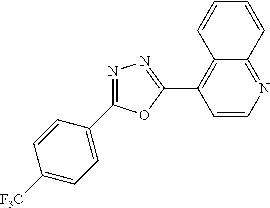 [SMILES: FC(F)(F)c1ccc(-c2nnc(-c3ccnc4ccccc34)o2)cc1] |
| 48 | 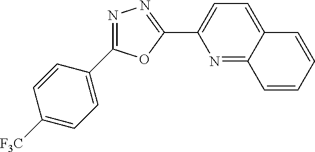 [SMILES: FC(F)(F)c1ccc(-c2nnc(-c3ccc4ccccc4n3)o2)cc1] |
| 49 | 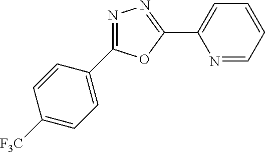 [SMILES: FC(F)(F)c1ccc(-c2nnc(-c3ccccn3)o2)cc1] |
| 50 | 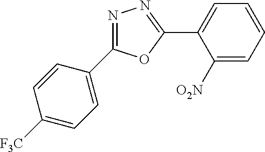 [SMILES: O=[N+]([O-])c1ccccc1-c1nnc(-c2ccc(C(F)(F)F)cc2)o1] |
| 51 | 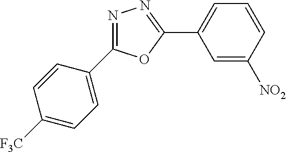 [SMILES: O=[N+]([O-])c1cccc(-c2nnc(-c3ccc(C(F)(F)F)cc3)o2)c1] |
| 52 | 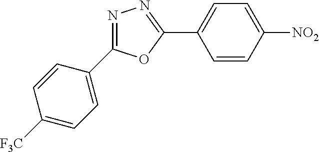 [SMILES: O=[N+]([O-])c1ccc(-c2nnc(-c3ccc(C(F)(F)F)cc3)o2)cc1] |
| 53 | 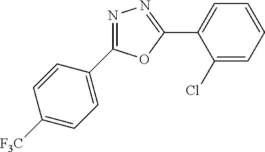 [SMILES: FC(F)(F)c1ccc(-c2nnc(-c3ccccc3Cl)o2)cc1] |
| 54 | 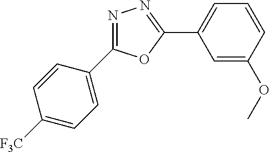 [SMILES: COc1cccc(-c2nnc(-c3ccc(C(F)(F)F)cc3)o2)c1] |
| 55 | 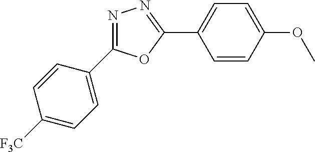 [SMILES: COc1ccc(-c2nnc(-c3ccc(C(F)(F)F)cc3)o2)cc1] |
| 56 | 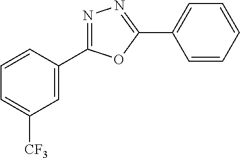 [SMILES: FC(F)(F)c1cccc(-c2nnc(-c3ccccc3)o2)c1] |
| 57 | 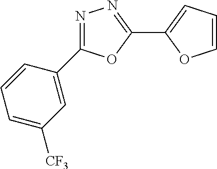 [SMILES: FC(F)(F)c1cccc(-c2nnc(-c3ccco3)o2)c1] |
| 58 | 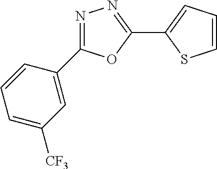 [SMILES: FC(F)(F)c1cccc(-c2nnc(-c3cccs3)o2)c1] |
| 59 | 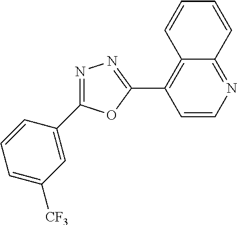 [SMILES: FC(F)(F)c1cccc(-c2nnc(-c3ccnc4ccccc34)o2)c1] |
| 60 | 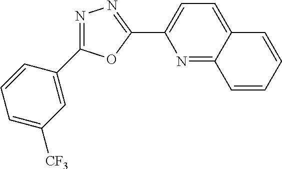 [SMILES: FC(F)(F)c1cccc(-c2nnc(-c3ccc4ccccc4n3)o2)c1] |
| 61 | 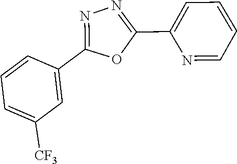 [SMILES: FC(F)(F)c1cccc(-c2nnc(-c3ccccn3)o2)c1] |
| 62 | 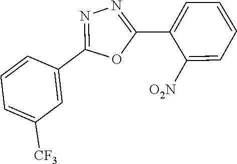 [SMILES: O=[N+]([O-])c1ccccc1-c1nnc(-c2cccc(C(F)(F)F)c2)o1] |
| 63 | 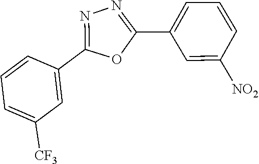 [SMILES: O=[N+]([O-])c1cccc(-c2nnc(-c3cccc(C(F)(F)F)c3)o2)c1] |
| 64 | 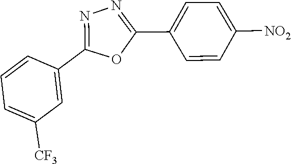 [SMILES: O=[N+]([O-])c1ccc(-c2nnc(-c3cccc(C(F)(F)F)c3)o2)cc1] |
| 65 | 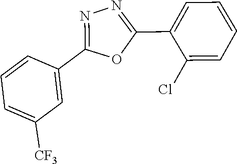 [SMILES: FC(F)(F)c1cccc(-c2nnc(-c3ccccc3Cl)o2)c1] |
| 66 | 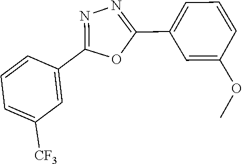 [SMILES: COc1cccc(-c2nnc(-c3cccc(C(F)(F)F)c3)o2)c1] |
| 67 | 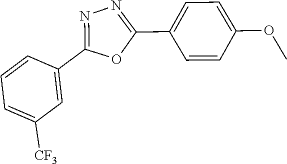 [SMILES: COc1ccc(-c2nnc(-c3cccc(C(F)(F)F)c3)o2)cc1] |
| 68 | 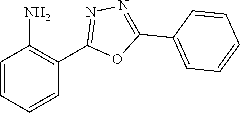 [SMILES: Nc1ccccc1-c1nnc(-c2ccccc2)o1] |
| 69 | 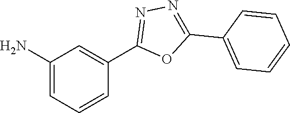 [SMILES: Nc1cccc(-c2nnc(-c3ccccc3)o2)c1] |
| 70 | 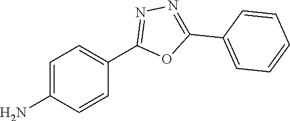 [SMILES: Nc1ccc(-c2nnc(-c3ccccc3)o2)cc1] |
| 71 | 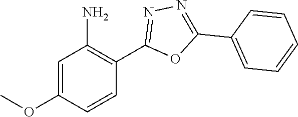 [SMILES: COc1ccc(-c2nnc(-c3ccccc3)o2)c(N)c1] |
| 72 | 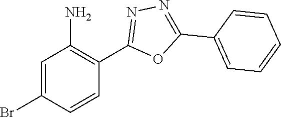 [SMILES: Nc1cc(Br)ccc1-c1nnc(-c2ccccc2)o1] |
| 73 | 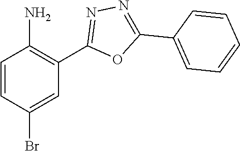 [SMILES: Nc1ccc(Br)cc1-c1nnc(-c2ccccc2)o1] |
| 74 | 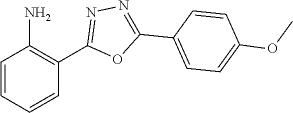 [SMILES: COc1ccc(-c2nnc(-c3ccccc3N)o2)cc1] |
| 75 | 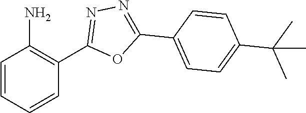 [SMILES: CC(C)(C)c1ccc(-c2nnc(-c3ccccc3N)o2)cc1] |
| 76 | 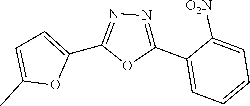 [SMILES: Cc1ccc(-c2nnc(-c3ccccc3[N+](=O)[O-])o2)o1] |
| 77 | 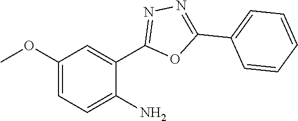 [SMILES: COc1ccc(N)c(-c2nnc(-c3ccccc3)o2)c1] |
| 78 | 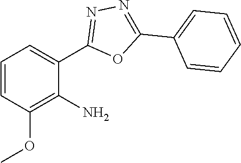 [SMILES: COc1cccc(-c2nnc(-c3ccccc3)o2)c1N] |
| 79 | 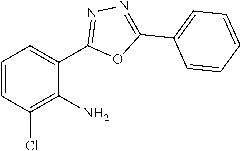 [SMILES: Nc1c(Cl)cccc1-c1nnc(-c2ccccc2)o1] |
| 80 | 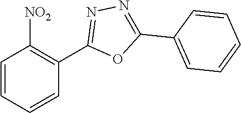 [SMILES: O=[N+]([O-])c1ccccc1-c1nnc(-c2ccccc2)o1] |
| 81 | 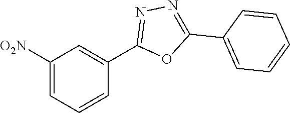 [SMILES: O=[N+]([O-])c1cccc(-c2nnc(-c3ccccc3)o2)c1] |
| 82 | 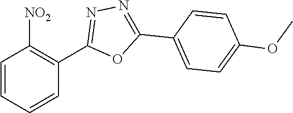 [SMILES: COc1ccc(-c2nnc(-c3ccccc3[N+](=O)[O-])o2)cc1] |
| 83 | 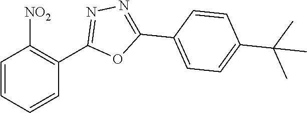 [SMILES: CC(C)(C)c1ccc(-c2nnc(-c3ccccc3[N+](=O)[O-])o2)cc1] |
| 84 | 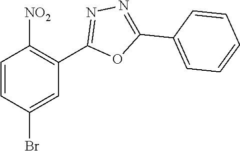 [SMILES: O=[N+]([O-])c1ccc(Br)cc1-c1nnc(-c2ccccc2)o1] |
| 85 | 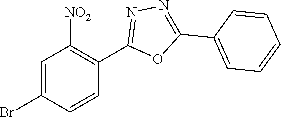 [SMILES: O=[N+]([O-])c1cc(Br)ccc1-c1nnc(-c2ccccc2)o1] |
| 86 | 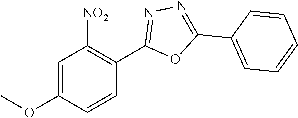 [SMILES: COc1ccc(-c2nnc(-c3ccccc3)o2)c([N+](=O)[O-])c1] |
| 87 | 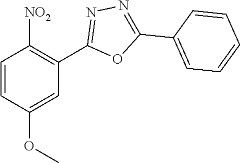 [SMILES: COc1ccc([N+](=O)[O-])c(-c2nnc(-c3ccccc3)o2)c1] |
| 88 | 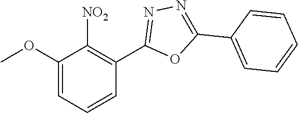 [SMILES: COc1cccc(-c2nnc(-c3ccccc3)o2)c1[N+](=O)[O-]] |
| 89 | 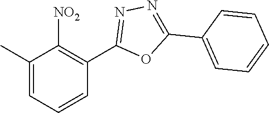 [SMILES: Cc1cccc(-c2nnc(-c3ccccc3)o2)c1[N+](=O)[O-]] |
| 90 | 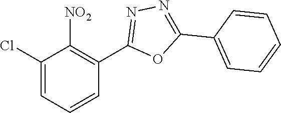 [SMILES: O=[N+]([O-])c1c(Cl)cccc1-c1nnc(-c2ccccc2)o1] |
| 91 | 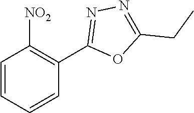 [SMILES: CCc1nnc(-c2ccccc2[N+](=O)[O-])o1] |
| 92 | 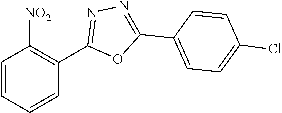 [SMILES: O=[N+]([O-])c1ccccc1-c1nnc(-c2ccc(Cl)cc2)o1] |
| 93 | 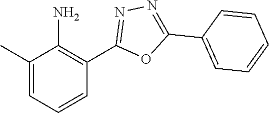 [SMILES: Cc1cccc(-c2nnc(-c3ccccc3)o2)c1N] |
| 94 | 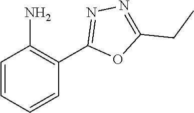 |
| 95 | 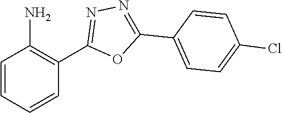 [SMILES: Nc1ccccc1-c1nnc(-c2ccc(Cl)cc2)o1] |
| 96 | 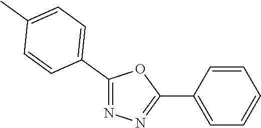 [SMILES: Cc1ccc(-c2nnc(-c3ccccc3)o2)cc1] |
| 97 | 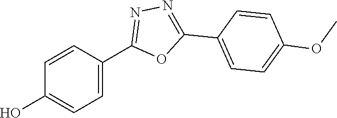 [SMILES: COc1ccc(-c2nnc(-c3ccc(O)cc3)o2)cc1] |
| 98 | 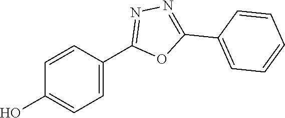 [SMILES: Oc1ccc(-c2nnc(-c3ccccc3)o2)cc1] |
| 99 | 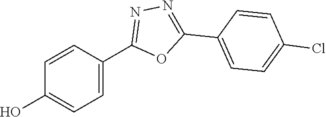 [SMILES: Oc1ccc(-c2nnc(-c3ccc(Cl)cc3)o2)cc1] |
| 100 | 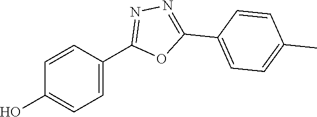 [SMILES: Cc1ccc(-c2nnc(-c3ccc(O)cc3)o2)cc1] |
| 101 | 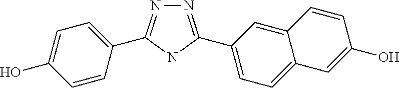 [SMILES: Oc1ccc(-c2nnc(-c3ccc4cc(O)ccc4c3)[nH]2)cc1] |
| 102 | 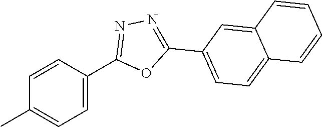 [SMILES: Cc1ccc(-c2nnc(-c3ccc4ccccc4c3)o2)cc1] |
| 103 | 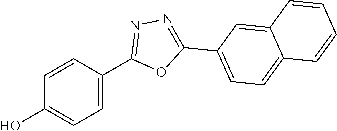 [SMILES: Oc1ccc(-c2nnc(-c3ccc4ccccc4c3)o2)cc1] |
| 104 | 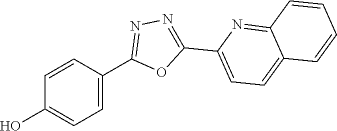 [SMILES: Oc1ccc(-c2nnc(-c3ccc4ccccc4n3)o2)cc1] |
| 105 | 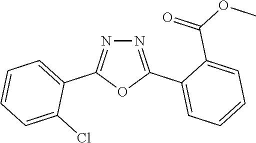 [SMILES: COC(=O)c1ccccc1-c1nnc(-c2ccccc2Cl)o1] |
| 106 | 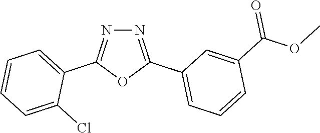 [SMILES: COC(=O)c1cccc(-c2nnc(-c3ccccc3Cl)o2)c1] |
| 107 | 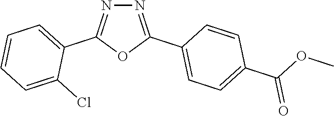 [SMILES: COC(=O)c1ccc(-c2nnc(-c3ccccc3Cl)o2)cc1] |
| 108 | 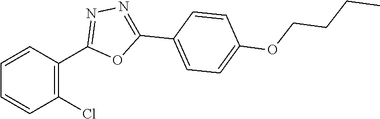 [SMILES: CCCCOc1ccc(-c2nnc(-c3ccccc3Cl)o2)cc1] |
| 109 | 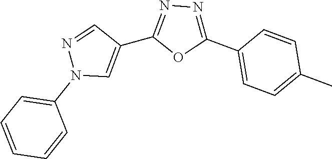 [SMILES: Cc1ccc(-c2nnc(-c3cnn(-c4ccccc4)c3)o2)cc1] |
| 110 | 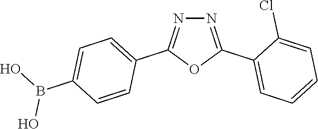 [SMILES: OB(O)c1ccc(-c2nnc(-c3ccccc3Cl)o2)cc1] |
| 111 | 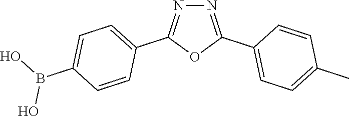 [SMILES: Cc1ccc(-c2nnc(-c3ccc(B(O)O)cc3)o2)cc1] |
| 112 | 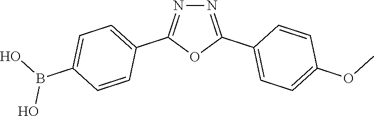 [SMILES: COc1ccc(-c2nnc(-c3ccc(B(O)O)cc3)o2)cc1] |
| 113 | 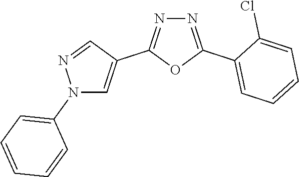 [SMILES: Clc1ccccc1-c1nnc(-c2cnn(-c3ccccc3)c2)o1] |
| 114 | 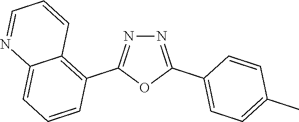 [SMILES: Cc1ccc(-c2nnc(-c3cccc4ncccc34)o2)cc1] |
| 115 | 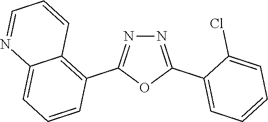 [SMILES: Clc1ccccc1-c1nnc(-c2cccc3ncccc23)o1] |
| 116 | 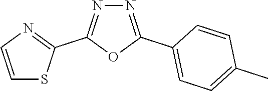 [SMILES: Cc1ccc(-c2nnc(-c3nccs3)o2)cc1] |
| 117 | 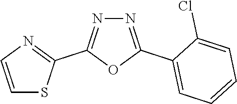 [SMILES: Clc1ccccc1-c1nnc(-c2nccs2)o1] |
| 118 | 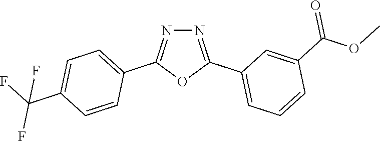 [SMILES: COC(=O)c1cccc(-c2nnc(-c3ccc(C(F)(F)F)cc3)o2)c1] |
| 119 | 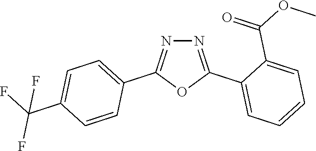 [SMILES: COC(=O)c1ccccc1-c1nnc(-c2ccc(C(F)(F)F)cc2)o1] |
| 120 | 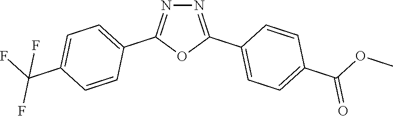 [SMILES: COC(=O)c1ccc(-c2nnc(-c3ccc(C(F)(F)F)cc3)o2)cc1] |
| 121 | 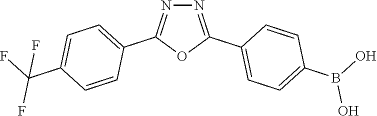 [SMILES: OB(O)c1ccc(-c2nnc(-c3ccc(C(F)(F)F)cc3)o2)cc1] |
| 122 | 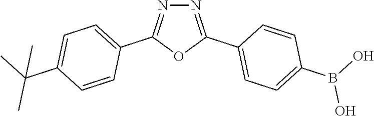 [SMILES: CC(C)(C)c1ccc(-c2nnc(-c3ccc(B(O)O)cc3)o2)cc1] |
| 123 | 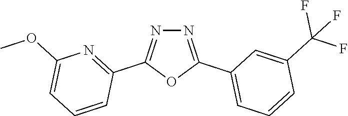 [SMILES: COc1cccc(-c2nnc(-c3cccc(C(F)(F)F)c3)o2)n1] |
| 124 | 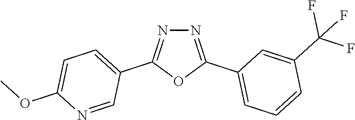 [SMILES: COc1ccc(-c2nnc(-c3cccc(C(F)(F)F)c3)o2)cn1] |
| 125 | 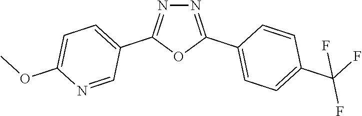 [SMILES: COc1ccc(-c2nnc(-c3ccc(C(F)(F)F)cc3)o2)cn1] |
| 126 | 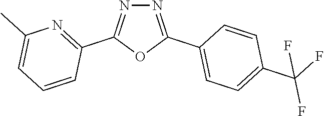 [SMILES: Cc1cccc(-c2nnc(-c3ccc(C(F)(F)F)cc3)o2)n1] |
| 127 | 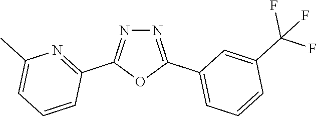 [SMILES: Cc1cccc(-c2nnc(-c3cccc(C(F)(F)F)c3)o2)n1] |
| 128 | 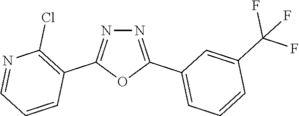 [SMILES: FC(F)(F)c1cccc(-c2nnc(-c3cccnc3Cl)o2)c1] |
| 129 | 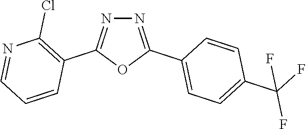 [SMILES: FC(F)(F)c1ccc(-c2nnc(-c3cccnc3Cl)o2)cc1] |
| 130 | 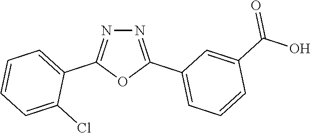 [SMILES: O=C(O)c1cccc(-c2nnc(-c3ccccc3Cl)o2)c1] |
| 131 | 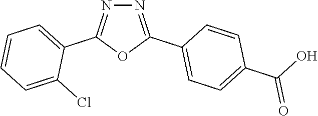 [SMILES: O=C(O)c1ccc(-c2nnc(-c3ccccc3Cl)o2)cc1] |
| 132 | 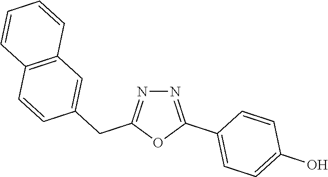 [SMILES: Oc1ccc(-c2nnc(Cc3ccc4ccccc4c3)o2)cc1] |
| 133 | 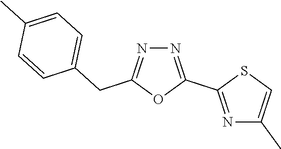 |
| 135 | 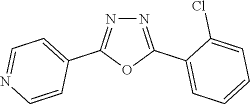 [SMILES: Clc1ccccc1-c1nnc(-c2ccncc2)o1] |
| 135 | 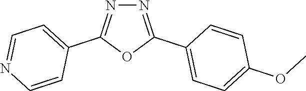 [SMILES: COc1ccc(-c2nnc(-c3ccncc3)o2)cc1] |
| 136 | 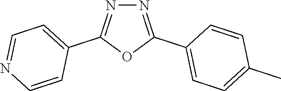 [SMILES: Cc1ccc(-c2nnc(-c3ccncc3)o2)cc1] |
| 137 | 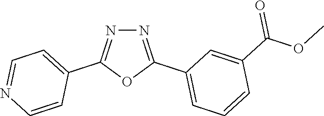 [SMILES: COC(=O)c1cccc(-c2nnc(-c3ccncc3)o2)c1] |
| 138 | 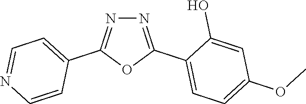 [SMILES: COc1ccc(-c2nnc(-c3ccncc3)o2)c(O)c1] |
| 139 | 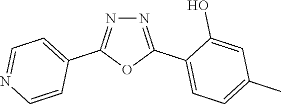 [SMILES: Cc1ccc(-c2nnc(-c3ccncc3)o2)c(O)c1] |
| 140 | 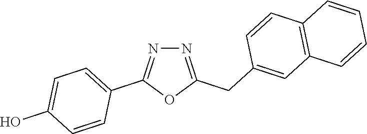 [SMILES: Oc1ccc(-c2nnc(Cc3ccc4ccccc4c3)o2)cc1] |
| 141 | 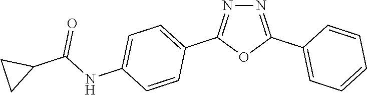 [SMILES: O=C(Nc1ccc(-c2nnc(-c3ccccc3)o2)cc1)C1CC1] |
| 142 | 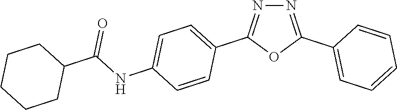 [SMILES: O=C(Nc1ccc(-c2nnc(-c3ccccc3)o2)cc1)C1CCCCC1] |
| 143 | 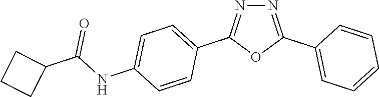 [SMILES: O=C(Nc1ccc(-c2nnc(-c3ccccc3)o2)cc1)C1CCC1] |
| 144 | 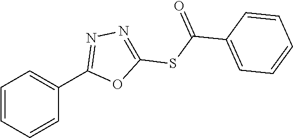 [SMILES: O=C(Sc1nnc(-c2ccccc2)o1)c1ccccc1] |
| 145 | 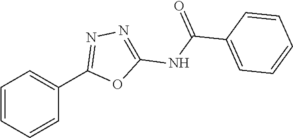 [SMILES: O=C(Nc1nnc(-c2ccccc2)o1)c1ccccc1] |
| 146 | 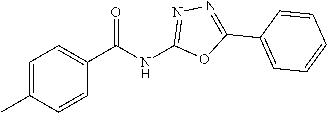 [SMILES: Cc1ccc(C(=O)Nc2nnc(-c3ccccc3)o2)cc1] |
| 147 | 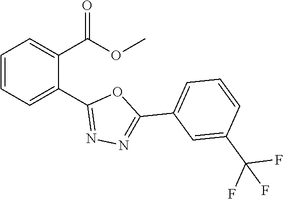 [SMILES: COC(=O)c1ccccc1-c1nnc(-c2cccc(C(F)(F)F)c2)o1] |
| 148 | 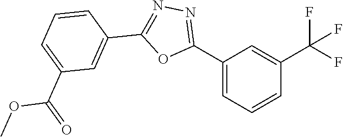 [SMILES: COC(=O)c1cccc(-c2nnc(-c3cccc(C(F)(F)F)c3)o2)c1] |
| 149 | 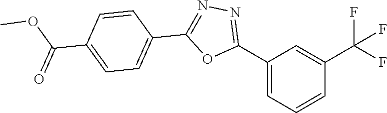 [SMILES: COC(=O)c1ccc(-c2nnc(-c3cccc(C(F)(F)F)c3)o2)cc1] |
| 150 | 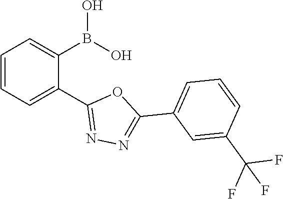 [SMILES: OB(O)c1ccccc1-c1nnc(-c2cccc(C(F)(F)F)c2)o1] |
| 151 | 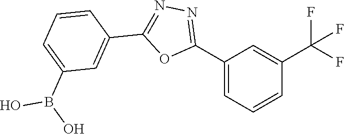 [SMILES: OB(O)c1cccc(-c2nnc(-c3cccc(C(F)(F)F)c3)o2)c1] |
| 152 | 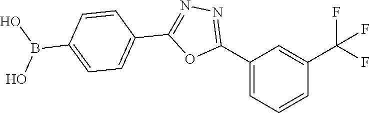 [SMILES: OB(O)c1ccc(-c2nnc(-c3cccc(C(F)(F)F)c3)o2)cc1] |
| 153 | 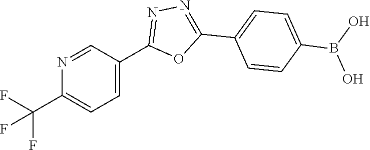 [SMILES: OB(O)c1ccc(-c2nnc(-c3ccc(C(F)(F)F)nc3)o2)cc1] |
| 154 | 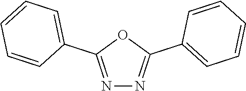 [SMILES: c1ccc(-c2nnc(-c3ccccc3)o2)cc1] |
| 155 | 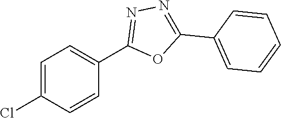 [SMILES: Clc1ccc(-c2nnc(-c3ccccc3)o2)cc1] |
| 156 | 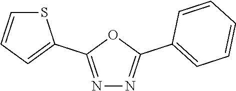 [SMILES: c1ccc(-c2nnc(-c3cccs3)o2)cc1] |
| 157 | 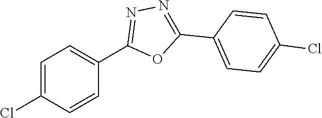 [SMILES: Clc1ccc(-c2nnc(-c3ccc(Cl)cc3)o2)cc1] |
| 158 | 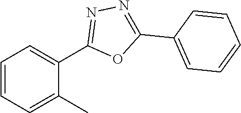 [SMILES: Cc1ccccc1-c1nnc(-c2ccccc2)o1] |
| 159 | 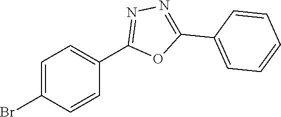 [SMILES: Brc1ccc(-c2nnc(-c3ccccc3)o2)cc1] |
| 160 | 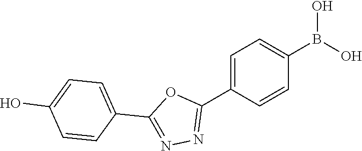 [SMILES: OB(O)c1ccc(-c2nnc(-c3ccc(O)cc3)o2)cc1] |

TABLE 2
Series B Compounds

| Entry | Structure |
|---|---|
| 1001 | 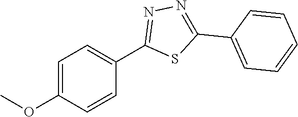 [SMILES: COc1ccc(-c2nnc(-c3ccccc3)s2)cc1] |
| 1002 | 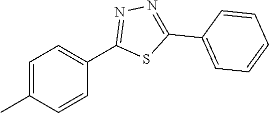 [SMILES: Cc1ccc(-c2nnc(-c3ccccc3)s2)cc1] |
| 1003 | 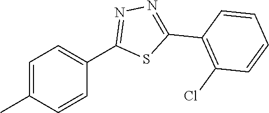 [SMILES: Cc1ccc(-c2nnc(-c3ccccc3Cl)s2)cc1] |
| 1004 | 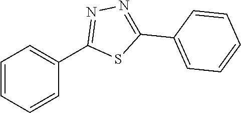 [SMILES: c1ccc(-c2nnc(-c3ccccc3)s2)cc1] |
| 1005 | 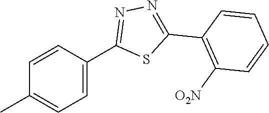 [SMILES: Cc1ccc(-c2nnc(-c3ccccc3[N+](=O)[O-])s2)cc1] |
| 1006 | 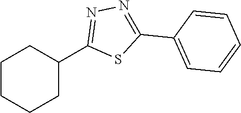 [SMILES: c1ccc(-c2nnc(C3CCCCC3)s2)cc1] |
| 1007 | 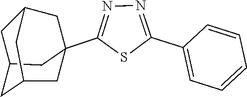 [SMILES: c1ccc(-c2nnc(C34CC5CC(CC(C5)C3)C4)s2)cc1] |
| 1008 | 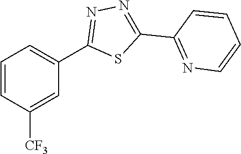 [SMILES: FC(F)(F)c1cccc(-c2nnc(-c3ccccn3)s2)c1] |
| 1009 | 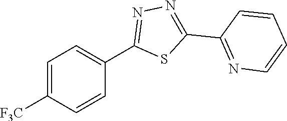 [SMILES: FC(F)(F)c1ccc(-c2nnc(-c3ccccn3)s2)cc1] |
| 1010 | 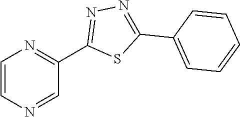 [SMILES: c1ccc(-c2nnc(-c3cnccn3)s2)cc1] |
| 1011 | 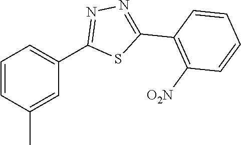 [SMILES: Cc1cccc(-c2nnc(-c3ccccc3[N+](=O)[O-])s2)c1] |
| 1012 | 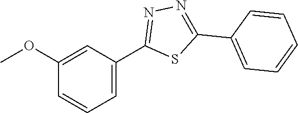 [SMILES: COc1cccc(-c2nnc(-c3ccccc3)s2)c1] |
| 1013 | 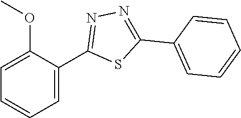 [SMILES: COc1ccccc1-c1nnc(-c2ccccc2)s1] |
| 1014 | 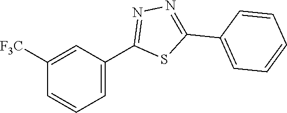 [SMILES: FC(F)(F)c1cccc(-c2nnc(-c3ccccc3)s2)c1] |
| 1015 | 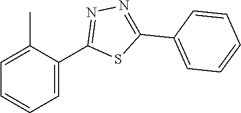 [SMILES: Cc1ccccc1-c1nnc(-c2ccccc2)s1] |
| 1016 | 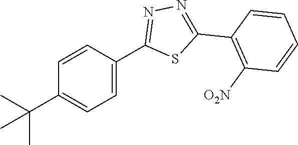 [SMILES: CC(C)(C)c1ccc(-c2nnc(-c3ccccc3[N+](=O)[O-])s2)cc1] |
| 1017 | 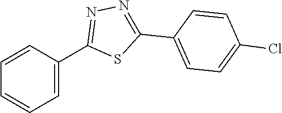 [SMILES: Clc1ccc(-c2nnc(-c3ccccc3)s2)cc1] |
| 1018 | 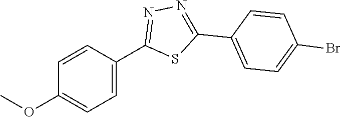 [SMILES: COc1ccc(-c2nnc(-c3ccc(Br)cc3)s2)cc1] |
| 1019 | 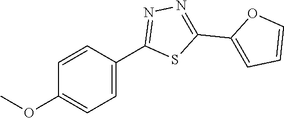 [SMILES: COc1ccc(-c2nnc(-c3ccco3)s2)cc1] |
| 1020 | 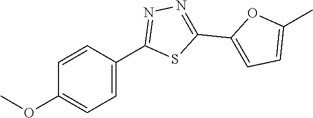 |
| 1021 | 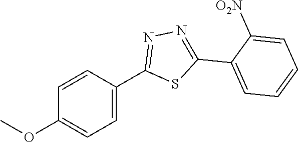 [SMILES: COc1ccc(-c2nnc(-c3ccccc3[N+](=O)[O-])s2)cc1] |
| 1022 | 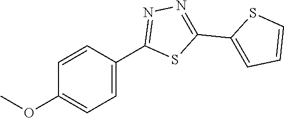 [SMILES: COc1ccc(-c2nnc(-c3cccs3)s2)cc1] |
| 1023 | 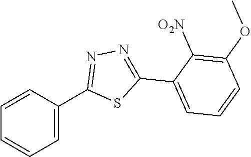 [SMILES: COc1cccc(-c2nnc(-c3ccccc3)s2)c1[N+](=O)[O-]] |
| 1024 | 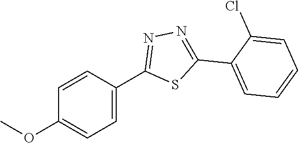 [SMILES: COc1ccc(-c2nnc(-c3ccccc3Cl)s2)cc1] |
| 1025 | 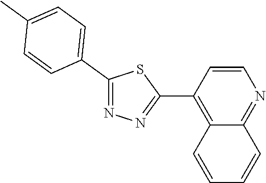 [SMILES: Cc1ccc(-c2nnc(-c3ccnc4ccccc34)s2)cc1] |
| 1026 | 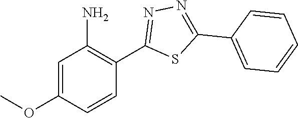 [SMILES: COc1ccc(-c2nnc(-c3ccccc3)s2)c(N)c1] |
| 1027 | 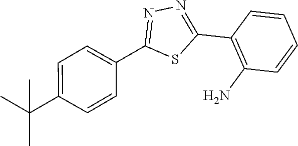 [SMILES: CC(C)(C)c1ccc(-c2nnc(-c3ccccc3N)s2)cc1] |
| 1028 | 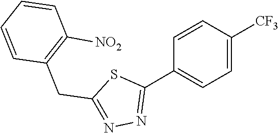 [SMILES: O=[N+]([O-])c1ccccc1Cc1nnc(-c2ccc(C(F)(F)F)cc2)s1] |
| 1029 | 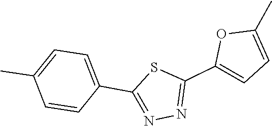 |
| 1030 | 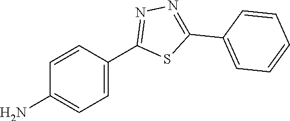 [SMILES: Nc1ccc(-c2nnc(-c3ccccc3)s2)cc1] |
| 1031 | 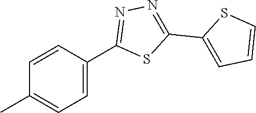 [SMILES: Cc1ccc(-c2nnc(-c3cccs3)s2)cc1] |
| 1032 | 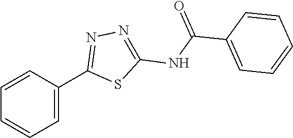 [SMILES: O=C(Nc1nnc(-c2ccccc2)s1)c1ccccc1] |
| 1033 | 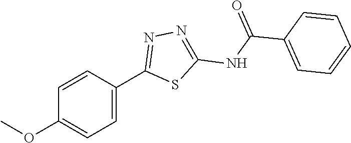 [SMILES: COc1ccc(-c2nnc(NC(=O)c3ccccc3)s2)cc1] |
| 1034 | 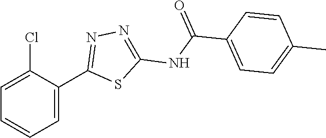 [SMILES: Cc1ccc(C(=O)Nc2nnc(-c3ccccc3Cl)s2)cc1] |
| 1035 | 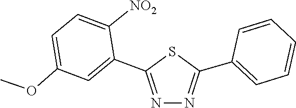 [SMILES: COc1ccc([N+](=O)[O-])c(-c2nnc(-c3ccccc3)s2)c1] |
| 1036 | 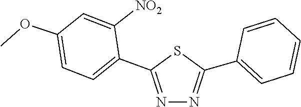 [SMILES: COc1ccc(-c2nnc(-c3ccccc3)s2)c([N+](=O)[O-])c1] |
| 1037 | 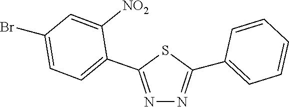 [SMILES: O=[N+]([O-])c1cc(Br)ccc1-c1nnc(-c2ccccc2)s1] |
| 1038 | 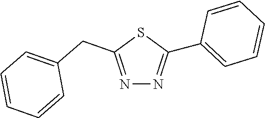 [SMILES: c1ccc(Cc2nnc(-c3ccccc3)s2)cc1] |
| 1039 | 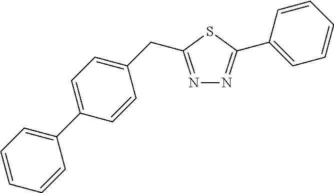 [SMILES: c1ccc(-c2ccc(Cc3nnc(-c4ccccc4)s3)cc2)cc1] |
| 1040 | 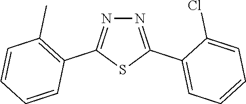 [SMILES: Cc1ccccc1-c1nnc(-c2ccccc2Cl)s1] |
| 1041 | 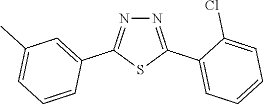 [SMILES: Cc1cccc(-c2nnc(-c3ccccc3Cl)s2)c1] |
| 1042 | 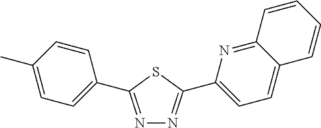 [SMILES: Cc1ccc(-c2nnc(-c3ccc4ccccc4n3)s2)cc1] |
| 1043 | 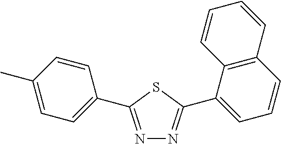 [SMILES: Cc1ccc(-c2nnc(-c3cccc4ccccc34)s2)cc1] |
| 1044 | 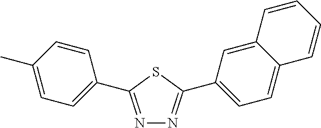 [SMILES: Cc1ccc(-c2nnc(-c3ccc4ccccc4c3)s2)cc1] |
| 1045 | 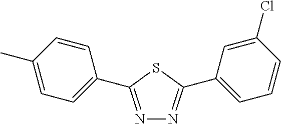 [SMILES: Cc1ccc(-c2nnc(-c3cccc(Cl)c3)s2)cc1] |
| 1046 | 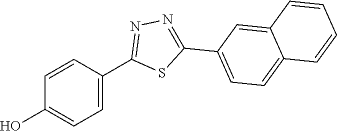 [SMILES: Oc1ccc(-c2nnc(-c3ccc4ccccc4c3)s2)cc1] |
| 1047 | 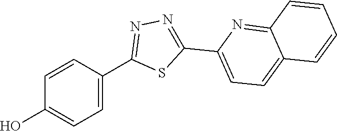 [SMILES: Oc1ccc(-c2nnc(-c3ccc4ccccc4n3)s2)cc1] |
| 1048 | 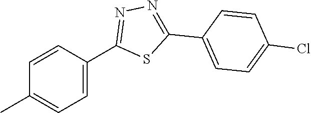 [SMILES: Cc1ccc(-c2nnc(-c3ccc(Cl)cc3)s2)cc1] |
| 1049 | 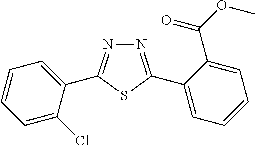 [SMILES: COC(=O)c1ccccc1-c1nnc(-c2ccccc2Cl)s1] |
| 1050 | 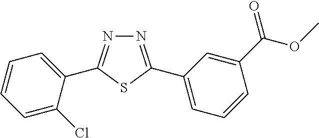 [SMILES: COC(=O)c1cccc(-c2nnc(-c3ccccc3Cl)s2)c1] |
| 1051 | 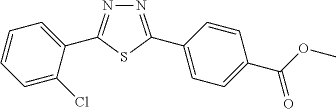 [SMILES: COC(=O)c1ccc(-c2nnc(-c3ccccc3Cl)s2)cc1] |
| 1052 | 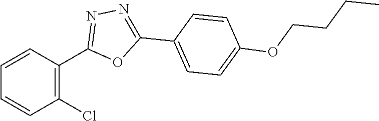 [SMILES: CCCCOc1ccc(-c2nnc(-c3ccccc3Cl)o2)cc1] |
| 1053 | 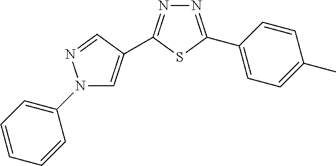 [SMILES: Cc1ccc(-c2nnc(-c3cnn(-c4ccccc4)c3)s2)cc1] |
| 1054 | 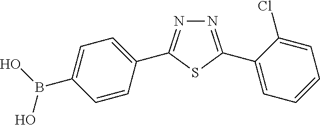 [SMILES: OB(O)c1ccc(-c2nnc(-c3ccccc3Cl)s2)cc1] |
| 1055 | 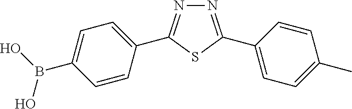 [SMILES: Cc1ccc(-c2nnc(-c3ccc(B(O)O)cc3)s2)cc1] |
| 1056 | 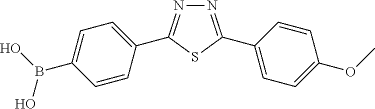 [SMILES: COc1ccc(-c2nnc(-c3ccc(B(O)O)cc3)s2)cc1] |
| 1057 | 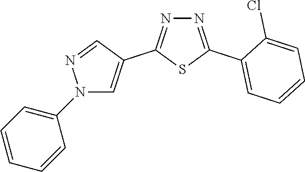 [SMILES: Clc1ccccc1-c1nnc(-c2cnn(-c3ccccc3)c2)s1] |
| 1058 | 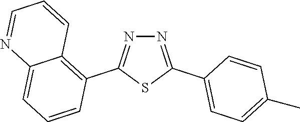 [SMILES: Cc1ccc(-c2nnc(-c3cccc4ncccc34)s2)cc1] |
| 1059 | 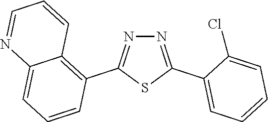 [SMILES: Clc1ccccc1-c1nnc(-c2cccc3ncccc23)s1] |
| 1060 | 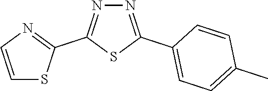 [SMILES: Cc1ccc(-c2nnc(-c3nccs3)s2)cc1] |
| 1061 | 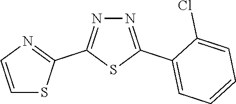 [SMILES: Clc1ccccc1-c1nnc(-c2nccs2)s1] |
| 1062 | 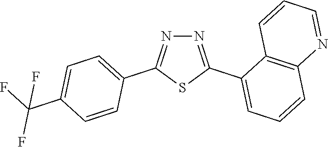 [SMILES: FC(F)(F)c1ccc(-c2nnc(-c3cccc4ncccc34)s2)cc1] |
| 1063 | 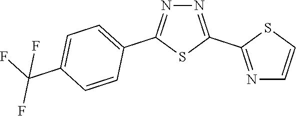 [SMILES: FC(F)(F)c1ccc(-c2nnc(-c3nccs3)s2)cc1] |
| 1064 | 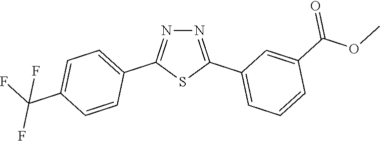 [SMILES: COC(=O)c1cccc(-c2nnc(-c3ccc(C(F)(F)F)cc3)s2)c1] |
| 1065 | 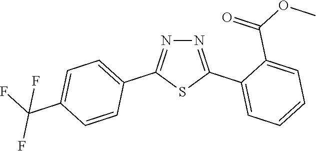 [SMILES: COC(=O)c1ccccc1-c1nnc(-c2ccc(C(F)(F)F)cc2)s1] |
| 1066 | 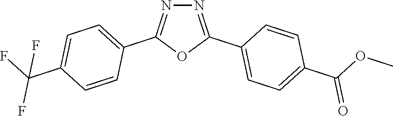 [SMILES: COC(=O)c1ccc(-c2nnc(-c3ccc(C(F)(F)F)cc3)o2)cc1] |
| 1067 | 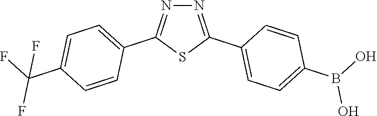 [SMILES: OB(O)c1ccc(-c2nnc(-c3ccc(C(F)(F)F)cc3)s2)cc1] |
| 1068 | 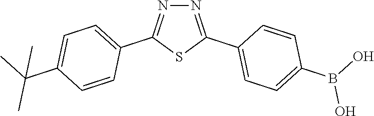 [SMILES: CC(C)(C)c1ccc(-c2nnc(-c3ccc(B(O)O)cc3)s2)cc1] |
| 1069 | 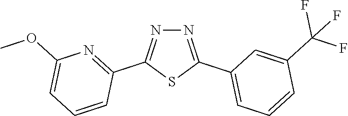 [SMILES: COc1cccc(-c2nnc(-c3cccc(C(F)(F)F)c3)s2)n1] |
| 1070 | 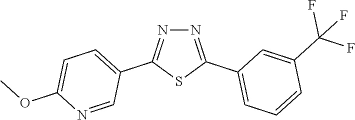 [SMILES: COc1ccc(-c2nnc(-c3cccc(C(F)(F)F)c3)s2)cn1] |
| 1071 | 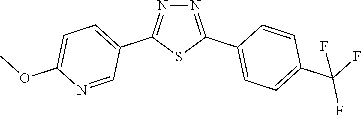 [SMILES: COc1ccc(-c2nnc(-c3ccc(C(F)(F)F)cc3)s2)cn1] |
| 1072 | 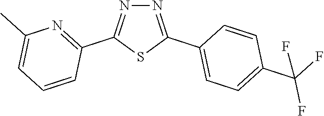 [SMILES: Cc1cccc(-c2nnc(-c3ccc(C(F)(F)F)cc3)s2)n1] |
| 1073 | 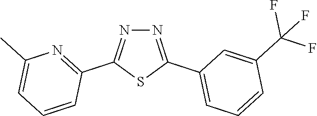 [SMILES: Cc1cccc(-c2nnc(-c3cccc(C(F)(F)F)c3)s2)n1] |
| 1074 | 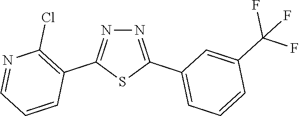 [SMILES: FC(F)(F)c1cccc(-c2nnc(-c3cccnc3Cl)s2)c1] |
| 1075 | 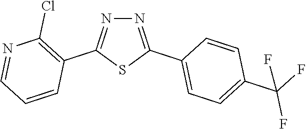 [SMILES: FC(F)(F)c1ccc(-c2nnc(-c3cccnc3Cl)s2)cc1] |
| 1076 | 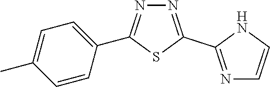 [SMILES: Cc1ccc(-c2nnc(-c3ncc[nH]3)s2)cc1] |
| 1077 | 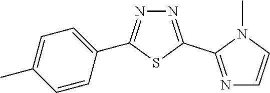 [SMILES: Cc1ccc(-c2nnc(-c3nccn3C)s2)cc1] |
| 1078 | 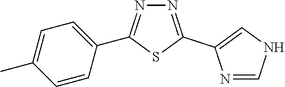 [SMILES: Cc1ccc(-c2nnc(-c3c[nH]cn3)s2)cc1] |
| 1079 | 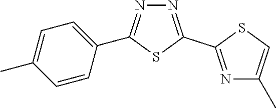 |
| 1080 | 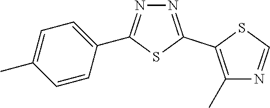 [SMILES: Cc1ccc(-c2nnc(-c3scnc3C)s2)cc1] |
| 1081 | 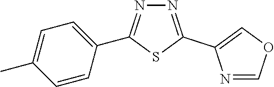 [SMILES: Cc1ccc(-c2nnc(-c3cocn3)s2)cc1] |
| 1082 | 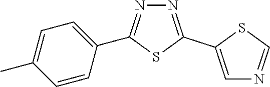 [SMILES: Cc1ccc(-c2nnc(-c3cncs3)s2)cc1] |
| 1083 | 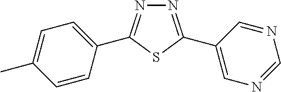 [SMILES: Cc1ccc(-c2nnc(-c3cncnc3)s2)cc1] |
| 1084 | 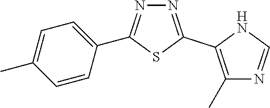 [SMILES: Cc1ccc(-c2nnc(-c3[nH]cnc3C)s2)cc1] |
| 1085 | 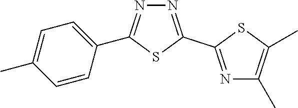 |
| 1086 | 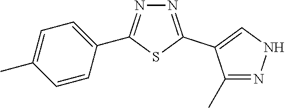 [SMILES: Cc1ccc(-c2nnc(-c3c[nH]nc3C)s2)cc1] |
| 1087 | 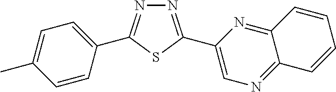 [SMILES: Cc1ccc(-c2nnc(-c3cnc4ccccc4n3)s2)cc1] |
| 1088 | 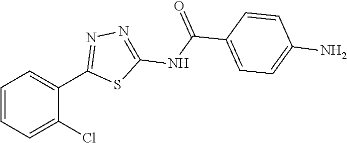 [SMILES: Nc1ccc(C(=O)Nc2nnc(-c3ccccc3Cl)s2)cc1] |

All the ERR modulating compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the ERR modulating compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the United States Food and Drug Administration (FDA). In the United States, the FDA is responsible for protecting the public health by assuring the safety, effectiveness, quality, and security of human and veterinary drugs, vaccines and other biological products, and medical devices.

In some embodiments, the ERR modulating compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the art, whether for use in the indications stated herein or otherwise.

The ERR modulating compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the ERR modulating compounds of the present disclosure can have the S or the R configuration. In some embodiments, the present compounds may contain two or more atoms which have a defined stereochemical orientation.

Chemical formulas used to represent the ERR modulating compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the ERR modulating compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, the ERR modulating compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclosure contemplates prodrugs of the ERR modulating compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, the ERR modulating compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the ERR modulating compounds provided herein, including any solvates thereof are within the scope of the present invention.

### B. Formulations

In some embodiments of the present disclosure, the ERR modulating compounds of the present disclosure are included a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the compounds described herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### II. METABOLIC DISEASES

Metabolic diseases include those caused by a wide range of metabolic defects, with varying manifestations. For example, obesity and diabetes are metabolic disorders that may be linked or be found separately. Muscle wasting diseases, including various forms of muscular dystrophy, also are consider disorders with metabolic bases. Some of these conditions are described below.

In some embodiments, the condition is selected from the group consisting of non-alcoholic fatty liver disease, non-alcoholic steatohepatitis (NASH), hypothyroidism, obesity, monogenic obesity, diabetes type I, diabetes type II, and lipodystrophy.

In some embodiments, the one or more symptoms of the disease or condition associated with metabolic dysfunction is selected from the group consisting of adiposity, obesity, hyperphagia, hyperglycemia, hypoleptinemia, hypertriglyceridemia, hypercholesterolemia, insulin resistance, dyslipidemia, delay in growth, delay in pubertal growth spurt, abnormal growth hormone secretion, elevated HbA1c, low bone mineral density (or low bone mass), low bone mineral content, and low lean body mass. The symptoms of the disease or condition associated with metabolic dysfunction can be prevented, ameliorated, or lessened in severity and/or duration, or reduced, following administration of a compound or composition of the disclosure that binds human ERR.

### A. Diabetes

Diabetes mellitus, often simply referred to as diabetes, is a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger).

### B. Obesity

Another aspect of the present disclosure concerns new methods and compounds for the treatment and prevention of obesity. Obesity is a medical condition in which excess body fat has accumulated to the extent that it may have an adverse effect on health. It is typically defined by body mass index (BMI) and may be further evaluated in terms of fat distribution via the waist-hip ratio and total cardiovascular risk factors. BMI is related to both percentage body fat and total body fat. Obesity increases the risk of many physical and mental conditions. These comorbidities are most commonly shown in metabolic syndrome, a combination of medical disorders which includes: diabetes mellitus type 2, high blood pressure, high blood cholesterol, and high triglyceride levels.

In some embodiments, the disclosure includes a method of treating or lessening the severity of a disease or disorder in a subject comprising administering to the subject a compound or pharmaceutical composition of the disclosure, wherein the disease or disorder is obesity.

In some embodiments, the treatment reverses or mitigates one or more of hyperphagia, obesity, hyperinsulinemia, dyslipidemia, and hepatosteatosis in the subject. In some embodiments, the subject's blood glucose is decreased, the subject's body weight is decreased, the subject exhibits decreased food intake, the subject's fat mass is decreased, the subject's lean mass is increased, and/or the subject's bone mass is increased.

In other aspects, provided herein are therapeutic methods for treating obesity or reducing body weight. In some aspects, provided herein are therapeutic methods for reducing fat body mass. In some aspects, provided herein are therapeutic methods of increasing lean body mass.

### C. Muscular Dystrophies

Muscular dystrophy (MD) is a group of muscle diseases that results in increasing weakening and breakdown of skeletal muscles over time. The disorders differ in which muscles are primarily affected, the degree of weakness, how fast they worsen, and when symptoms begin. Many people will eventually become unable to walk. Some types are also associated with problems in other organs.

### D. Non-Alcoholic Fatty Liver Disease

Non-alcoholic fatty liver disease (NAFLD) is one of the types of fatty liver which occurs when fat is deposited (steatosis) in the liver due to causes other than excessive alcohol use. Non-alcoholic steatohepatitis (NASH) is the most extreme form of NAFLD. NAFLD is the most common liver disorder in developed countries.

### Other Uses and Indications

In some embodiments, the compounds of Formula I may be used for treating one or more of: heart failure (including but not limited to heart failure with reduced ejection fraction and heart failure with preserved ejection fraction), cardiac fibrosis, cardiac hypertrophy, myopathy, dilated cardiac myopathy, heart failure, obesity, metabolic disease, muscle wasting and other diseases of muscle function, diseases of mitochongrial disfunction, kidney diseases and disfunction, chronic kidney disease, and neurodegenerative diseases including AD, PD, and ALS.

In some embodiments, the compounds of Formula I may be used for treating one or more of: heart failure (including but not limited to heart failure with reduced ejection fraction and heart failure with preserved ejection fraction), cardiac fibrosis, cardiac hypertrophy, myopathy, dilated cardiac myopathy, heart failure, obesity, metabolic disease, muscle wasting and other diseases of muscle function, diseases of mitochongrial disfunction, kidney diseases and disfunction, chronic kidney disease, and neurodegenerative diseases including AD, PD, and ALS based upon various types of assays. In one aspect, an assay may measure an increase in mitochondrial biogenesis. In another aspect, an assay may measure the increase in oxidative metabolism. In another aspect, an assay may measure an increase in fatty acid oxidation. In yet another aspect, an assay may also measure an increase in expression of genes directing the biochemical pathways responsible for oxidative metabolism and fatty acid metabolism.

### III. THERAPIES

### A. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a the ERR modulating compounds of the present disclosure formulated with one or more excipients and/or drug carriers appropriate to the indicated route of administration. In some embodiments, the ERR modulating compounds disclosed herein are formulated in a manner amenable for the treatment of human and/or veterinary patients. In some embodiments, formulation comprises admixing or combining one or more of the ERR modulating compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the ERR modulating compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the ERR modulating compounds disclosed herein may be coated in a material to protect the compound from the action of acids and other natural conditions which may inactivate the compound. To administer the active compound by other than parenteral administration, it may be necessary to coat the ERR modulating compound with, or co-administer the ERR modulating compound with, a material to prevent its inactivation. In some embodiments, the active ERR modulating compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The ERR modulating compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The ERR modulating compounds disclosed herein can be administered orally, for example, with an inert diluent or an assimilable edible carrier. The ERR modulating compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic ERR modulating compound in the compositions and preparations may, of course, be varied. The amount of the therapeutic ERR modulating compound in such pharmaceutical formulations is such that a suitable dosage will be obtained.

The therapeutic ERR modulating compound may also be administered topically to the skin, eye, ear, or mucosal membranes. Administration of the therapeutic ERR modulating compound topically may include formulations of the ERR modulating compounds as a topical solution, lotion, cream, ointment, gel, foam, transdermal patch, or tincture. When the therapeutic ERR modulating compound is formulated for topical administration, the ERR modulating compound may be combined with one or more agents that increase the permeability of the ERR modulating compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic ERR modulating compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic ERR modulating compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic ERR modulating compound calculated to produce the desired therapeutic effect in association with the required pharmaceutical carrier. In some embodiments, the specification for the dosage unit forms of the disclosure are dictated by and directly dependent on (a) the unique characteristics of the therapeutic ERR modulating compound and the particular therapeutic effect to be achieved, and (b) the limitations inherent in the art of compounding such a therapeutic ERR modulating compound for the treatment of a selected condition in a patient. In some embodiments, active ERR modulating compounds are administered at a therapeutically effective dosage sufficient to treat a condition associated with a condition in a patient. For example, the efficacy of a ERR modulating compound can be evaluated in an animal model system that may be predictive of efficacy in treating the disease in a human or another animal.

In some embodiments, the effective dose range for the therapeutic ERR modulating compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of an ERR modulating compound of the present disclosure or composition comprising an ERR modulating compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active ERR modulating compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### B. Methods of Treatment

In particular, the compositions that may be used in treating a metabolic condition in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, amount of the ERR modulating compounds used is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the ERR modulating compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### C. Combination Therapies

It is envisioned that the ERR modulating compounds described herein may be used in combination therapies with one or more additional therapies or a compound which mitigates one or more of the side effects experienced by the patient. It is common in the field of medicine to combine therapeutic modalities. The following is a general discussion of therapies that may be used in conjunction with the therapies of the present disclosure.

To treat diseases or disorders using the methods and compositions of the present disclosure, one would generally contact a cell or a subject with an ERR modulating compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the compounds described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. A discussion of other potential therapies that may be used combination with the compounds of the present disclosure is presented elsewhere in this document.

Another embodiment of the disclosure relates to modulating estrogen related receptor activity in a biological sample or a subject, which method comprises administering to the subject, or contacting said biological sample with a compound of the disclosure, a pharmaceutically acceptable salt thereof, or a pharmaceutical composition thereof. Another embodiment of the disclosure relates to inhibiting estrogen related receptor activity in a biological sample or a subject, which method comprises administering to the subject, or contacting said biological sample with a compound of the disclosure, a pharmaceutically acceptable salt thereof, or a pharmaceutical composition thereof.

The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

Modulation of estrogen related receptor activity in a biological sample is useful for a variety of purposes that are known to one of skill in the art. Examples of such purposes include, but are not limited to, the study of estrogen related receptor in biological and pathological phenomena, and the comparative evaluation of new estrogen related receptor modulators.

Inhibition of estrogen related receptor activity in a biological sample is useful for a variety of purposes that are known to one of skill in the art. Examples of such purposes include, but are not limited to, the study of estrogen related receptor in biological and pathological phenomena, and the comparative evaluation of new estrogen related receptor inhibitors.

In some embodiments, an additional therapeutic agent is a hyperglycemia or diabetes drug. Hyperglycemia or diabetes drugs include, but are not limited to, insulin and insulin mimetics; PPAR (peroxisome proliferator-activated receptor) γ-agonists, such as pioglitazone, troglitazone, ciglitazone, rivoglitazone, rosiglitazone, and other 2,4-thiazolidinedione derivatives; DPP-4 inhibitors, such as sitagliptin (JANUVIA), vildagliptin, saxagliptin, linagliptin (TRADJENTA), dutogliptin, gemigliptin, and alogliptin (NESINA); GLP-1 analogs, such as exenatide, liraglutide, taspoglutide, albiglutide, and lixisenatide; biguanidine derivatives, such as metformin (GLU-METZA, GLUCOPHAGE), buformin, and phenformin; ATP-sensitive potassium channel modulators, such as mitiglinide, repaglinide, and nateglinide; sulfonylurea derivatives, such as tolbutamide, chlorpropamide, tolazamide, acetohexamide, glipizide, gliclazide, glimepiride, gliquidone, glibomuride, glisoxepid, glibenclamide, glisen-tide, glisolamide, glybuzole, and glyclopyramide;
a-glucosidase inhibitors, such as miglitol (GLYSET), acarbose (PRECOSE), and voglibose; and SGLT2 inhibitors, such as canagliflozin (INVOKANA), dapagliflozin (FARXIGA), and empagliflozin (JARDIANCE).

In some embodiments, an additional therapeutic agent is an obesity drug. Obesity drugs include, but are not limited to, orlistat (XENICAL), phentermine/topiramate (QSYMIA), lorcaserin (BELVIQ), naltrexone/bupropion (CONTRAVE) and liraglutide (SAXENDA).

In some embodiments, an additional therapeutic agent is a lipid-lowering drug or a cholesterol-lowering drug. Lipid-lowering drugs include, but are not limited to, fibrates, statins, omega-3 fatty acids, and niacin. In some embodiments, an additional therapeutic agent is a fibrate. Fibrates are a class of amphipathic carboxylic acids and include, but are not limited to, aluminum clofibrate, bezafibrate, cipro-fibrate, choline fenofibrae, clinofibrate, clofibrate (e.g., ATROMID-S), clofibride, fenofibrate (e.g, FIBRICOR, LOFIBRA, TRICOR), gemfibrozil (e.g., LOPID), ronifi-brate, simfibrate, and fenofibric acid. In some embodiments, an additional therapeutic agent is a statin. Statins are HMG-CoA reductase inhibitors and include, but are not limited to, atorvastatin (LIPITOR), fluvastatin (LESCOL), lovastatin (MEVACOR), pravastatin (PRAVACHOL), rosuvastatin (ZOCOR), and pitavastatin (LIVALO). In some embodiments, the additional therapeutic agent is niacin (vitamin B3). In some embodiments, the additional therapeutic agent is an omega-3 fatty acid.

In some embodiments, an additional therapeutic agent is selected from the group including, but to limited to, glucagon receptor antagonists; GLP-1, GLP-1 mimetics, and GLP-1 receptor agonists; GIP, GIP mimetics, and GIP receptor agonists; PACAP, PACAP mimetics, and PACAP receptor 3 agonists; cholesterol-lowering agents such as HMG-CoA reductase inhibitors, sequestrants, nicotinyl alcohol, nicotinic acid and salts thereof, PPAR alpha agonists, PPAR alpha/gamma dual agonists, inhibitors of cholesterol absorption, acyl CoA: cholesterol acyltransferase inhibitors, anti-oxidants, and LXR modulators; PPAR delta agonists; anti-obesity compounds; ileal bile acid transporter inhibitors; anti-inflammatory agents excluding glucocorticoids; protein tyrosine phosphatase-IB (PTP-IB) inhibitors, and CB1 antagonists/inverse agonists.

### IV. CHEMISTRY BACKGROUND

In some aspects, the ERR modulating compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

### A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the compounds described herein.

### B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “ [symbol] ” means triple bond. The symbol “ [symbol] ” represents an optional bond, which if present is either single or double. The symbol “ [symbol] ” represents a single bond or a double bond. Thus, the formula

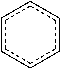

covers, for example,

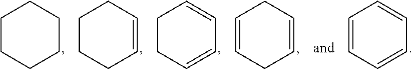

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

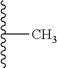

for methyl) indicates a point of attachment of the group. The point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

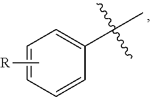

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

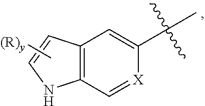

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl(C₁₋₆). Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

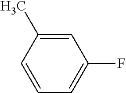

is also taken to refer to

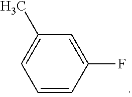

Aromatic compounds may also be depicted using a circle to represent the delocalized nature of the electrons in the fully conjugated cyclic π system, two non-limiting examples of which are shown below;

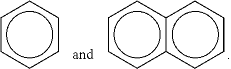

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

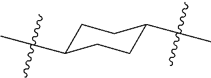 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

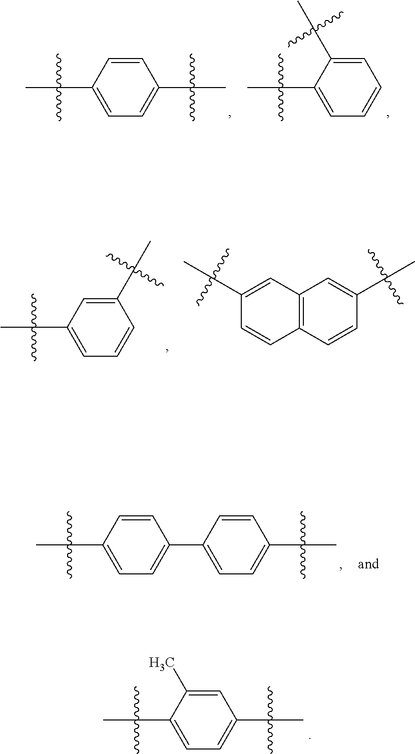

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkalkyl” refers to the monovalent group -alkanediyl-heterocycloalkyl, in which the terms alkanediyl and heterocycloalkyl are each used in a manner consistent with the definitions provided above. Non-limiting examples are: morpholinylmethyl and piperidinylethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of ±10% of the reported value, preferably a value of ±5% of the reported value. It is to be understood that, whenever the term “about” is used, a specific reference to the exact numerical value indicated is also included.”

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to affect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half. The term “EC₅₀” refers to an amount that is an effective concentration to results in a half-maximal response.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity within its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-is-p-hydroxynaphthoate, gentisates, isethionates, di-p-toluoyltartrates, methane-sulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. ADDITIONAL EMBODIMENTS (STATEMENTS)

Statement 1. A method of modulating the activity of an estrogen related receptor comprising contacting the estrogen related receptor with a compound of the formula:

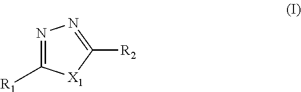

wherein:
   X₁ is O or S wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

Statement 2. The method of statement 1, wherein the compound is further defined as:

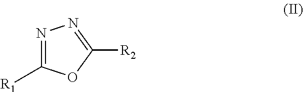

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

Statement 3. The method of statement 1, wherein the compound is further defined as:

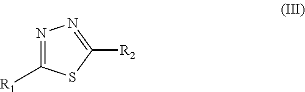

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

Statement 4. The method of statement 1, wherein X₁ is O.

Statement 5. The method of statement 1, wherein X₁ is S.

Statement 6. The method according to any one of statements 1-5, wherein R₁ is cycloalkyl_{(C≤12)} or substituted cycloalkyl_{(C≤12)}.

Statement 7. The method of statement 6, wherein R₁ is cycloalkyl_{(C≤12)}.

Statement 8. The method of statement 7, wherein R₁ is cyclohexyl or adamantyl.

Statement 9. The method according to any one of statements 1-5, wherein R₁ is aralkyl_{(C≤12)} or substituted aralkyl_{(C≤12)}.

Statement 10. The method of statement 9, wherein R₁ is substituted aralkyl_{(C≤12)}.

Statement 11. The method of statement 10, wherein R₁ is 2-nitrobenzyl.

Statement 12. The method according to any one of statements 1-5, wherein R₁ is aryl_{(C≤12)} or substituted aryl_{(C≤12)}.

Statement 13. The method of statement 12, wherein R₁ is aryl_{(C≤12)}.

Statement 14. The method of statement 13, wherein R₁ is phenyl or 4-methylphenyl.

Statement 15. The method of statement 12, wherein R₁ is substituted aryl_{(C≤12)}.

Statement 16. The method of statement 15, wherein R₁ is 4-bromophenyl, 4-chlorophenyl, 2-nitrophenyl, 3-nitrophenyl, 4-nitrophenyl, 4-methoxyphenyl, 3-trifluoromethylphenyl, 4-trifluoromethylphenyl, 2-aminophenyl, 3-aminophenyl, 4-aminophenyl, 2-amino-4-methoxyphenyl, 2-amino-4-bromophenyl, 2-amino-5-bromophenyl, 2-amino-5-methoxyphenyl, 2-amino-3-methoxyphenyl, 2-amino-3-chlorophenyl, 2-nitro-5-bromophenyl, 2-nitro-4-bromophenyl, 2-nitro-4-methoxyphenyl, 2-nitro-5-methoxyphenyl, 2-nitro-3-methoxyphenyl, or 2-nitro-3-chlorophenyl.

Statement 17. The method according to any one of statements 1-16, wherein R₂ is alkyl_{(C≤12)} or substituted alkyl_{(C≤12)}.

Statement 18. The method of statement 17, wherein R₂ is alkyl_{(C≤12)}.

Statement 19. The method of statement 18, wherein R₂ is ethyl.

Statement 20. The method according to any one of statements 1-16, wherein R₂ is aryl_{(C≤12)} or substituted aryl_{(C≤12)}.

Statement 21. The method of statement 20, wherein R₂ is aryl_{(C≤12)}.

Statement 22. The method of statement 21, wherein R₂ is phenyl, 2-methylphenyl, 4-methylphenyl, or 4-t-butylphenyl.

Statement 23. The method of statement 20, wherein R₂ is substituted aryl_{(C≤12)}.

Statement 24. The method of statement 23, wherein R₂ is 2-nitrophenyl, 2-chlorophenyl, 4-chlorophenyl, 4-methoxyphenyl, 3-methoxyphenyl, 3-nitrophenyl, 4-dimethylaminophenyl, 4-trifluorophenyl, 4-bromophenyl, or 4-chlorophenyl.

Statement 25. The method according to any one of statements 1-16, wherein R₂ is heteroaryl_{(C≤12)} or substituted heteroaryl_{(C≤12)}.

Statement 26. The method of statement 25, wherein R₂ is heteroaryl_{(C≤12)}.

Statement 27. The method of statement 26, wherein R₂ is 2-pyrindinyl, 2-quinolinyl, 4-quinolinyl, furanyl, thiophenyl, or 5-methylfuranyl.

Statement 28. The method according to any one of statements 1-27, wherein the compound is further defined as one selected from Table 1, Table 2, and pharmaceutically acceptable salts thereof.

Statement 29. The method according to any one of statements 1-28, wherein the method is performed in vivo.

Statement 30. The method according to any one of statements 1-28, wherein the method is performed in vitro.

Statement 31. The method according to any one of statements 1-28, wherein the method is performed ex vivo.

Statement 32. The method according to any one of statements 1-31, wherein the method results in modulation of the estrogen related receptor is sufficient to treat a disease or disorder.

Statement 33. The method of statement 32, wherein the disease or disorder is associated with estrogen-related receptor α.

Statement 34. The method of statement 32, wherein the disease or disorder is associated with estrogen-related receptor 7.

Statement 35. The method according to any one of statements 32-34, wherein the disease or disorder is associated with cellular energy metabolism.

Statement 36. The method of statement 35, wherein the disease or disorder is associated with glucose metabolism.

Statement 37. The method of statement 36, wherein the disease or disorder is diabetes.

Statement 38. The method of statement 35, wherein the disease or disorder is associated with lipid metabolism.

Statement 39. The method according to any one of statements 32-34, wherein the disease or disorder is cancer.

Statement 40. The method of statement 39, wherein the disease or disorder is breast cancer.

Statement 41. The method according to any one of statements 32-34, wherein the disease or disorder is a bone disease or disorder.

Statement 42. The method of statement 41, wherein the disease or disorder is associated with bone resorption.

Statement 43. A method of treating a disease or disorder in a patient in need thereof comprising administering to the patient a therapeutically effective amount of a compound of the formula:

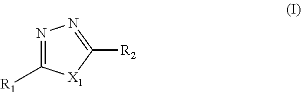

wherein:
   X₁ is O or S;
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

Statement 44. The method of statement 43, wherein the compound is further defined as:

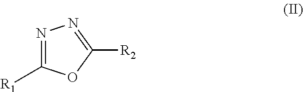

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

Statement 45. The method of statement 43, wherein the compound is further defined as:

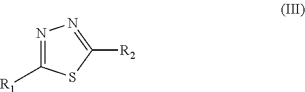

wherein:
   R₁ is cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups; and
   R₂ is alkyl_{(C≤12)}, cycloalkyl_{(C≤12)}, aryl_{(C≤12)}, aralkyl_{(C≤12)}, heteroaralkyl_{(C≤12)}, heteroaryl_{(C≤12)}, heterocycloalkyl_{(C≤12)}, heterocycloalkalkyl_{(C≤12)}, or a substituted version any of these groups;
or a pharmaceutically acceptable salt thereof.

Statement 46. The method of statement 43, wherein X₁ is O

Statement 47. The method of statement 43, wherein X₁ is S.

Statement 48. The method according to any one of statements 43-47, wherein R₁ is cycloalkyl_{(C≤12)} or substituted cycloalkyl_{(C≤12)}.

Statement 49. The method of statement 48, wherein R₁ is cycloalkyl_{(C≤12)}.

Statement 50. The method of statement 49, wherein R₁ is cyclohexyl or adamantyl.

Statement 51. The method according to any one of statements 43-47, wherein R₁ is aralkyl_{(C≤12)} or substituted aralkyl_{(C≤12)}.

Statement 52. The method of statement 51, wherein R₁ is substituted aralkyl_{(C≤12)}.

Statement 53. The method of statement 52, wherein R₁ is 2-nitrobenzyl.

Statement 54. The method according to any one of statements 43-47, wherein R₁ is aryl_{(C≤12)} or substituted aryl_{(C≤12)}.

Statement 55. The method of statement 54, wherein R₁ is aryl_{(C≤12)}.

Statement 56. The method of statement 55, wherein R₁ is phenyl or 4-methylphenyl.

Statement 57. The method of statement 54, wherein R₁ is substituted aryl_{(C≤12)}.

Statement 58. The method of statement 57, wherein R₁ is 4-bromophenyl, 4-chlorophenyl, 2-nitrophenyl, 3-nitrophenyl, 4-nitrophenyl, 4-methoxyphenyl, 3-trifluoromethylphenyl, 4-trifluoromethylphenyl, 2-aminophenyl, 3-aminophenyl, 4-aminophenyl, 2-amino-4-methoxyphenyl, 2-amino-4-bromophenyl, 2-amino-5-bromophenyl, 2-amino-5-methoxyphenyl, 2-amino-3-methoxyphenyl, 2-amino-3-chlorophenyl, 2-nitro-5-bromophenyl, 2-nitro-4-bromophenyl, 2-nitro-4-methoxyphenyl, 2-nitro-5-methoxyphenyl, 2-nitro-3-methoxyphenyl, or 2-nitro-3-chlorophenyl.

Statement 59. The method according to any one of statements 43-58, wherein R₂ is alkyl_{(C≤12)} or substituted alkyl_{(C≤12)}.

Statement 60. The method of statement 59, wherein R₂ is alkyl_{(C≤12)}.

Statement 61. The method of statement 60, wherein R₂ is ethyl.

Statement 62. The method according to any one of statements 43-58, wherein R₂ is aryl_{(C≤12)} or substituted aryl_{(C≤12)}.

Statement 63. The method of statement 62, wherein R₂ is aryl_{(C≤12)}.

Statement 64. The method of statement 63, wherein R₂ is phenyl, 2-methylphenyl, 4-methylphenyl, or 4-t-butylphenyl.

Statement 65. The method of statement 62, wherein R₂ is substituted aryl_{(C≤12)}.

Statement 66. The method of statement 65, wherein R₂ is 2-nitrophenyl, 2-chlorophenyl, 4-chlorophenyl, 4-methoxyphenyl, 3-methoxyphenyl, 3-nitrophenyl, 4-dimethylaminophenyl, 4-trifluorophenyl, 4-bromophenyl, or 4-chlorophenyl.

Statement 67. The method according to any one of statements 43-58, wherein R₂ is heteroaryl_{(C≤12)} or substituted heteroaryl_{(C≤12)}.

Statement 68. The method of statement 67, wherein R₂ is heteroaryl_{(C≤12)}.

Statement 69. The method of statement 68, wherein R₂ is 2-pyrindinyl, 2-quinolinyl, 4-quinolinyl, furanyl, thiophenyl, or 5-methylfuranyl.

Statement 70. The method according to any one of statements 43-69, wherein the compound is further defined as one selected from Table 1, Table 2, and pharmaceutically acceptable salts thereof.

Statement 71. The method according to any one of statements 43-70, wherein the disease or disorder is associated with estrogen-related receptor α.

Statement 72. The method according to any one of statements 43-70, wherein the disease or disorder is associated with estrogen-related receptor γ.

Statement 73. The method according to any one of statements 43-72, wherein the disease or disorder is associated with cellular energy metabolism.

Statement 74. The method of statement 73, wherein the disease or disorder is associated with glucose metabolism.

Statement 75. The method of statement 74, wherein the disease or disorder is diabetes.

Statement 76. The method of statement 73, wherein the disease or disorder is associated with lipid metabolism.

Statement 77. The method according to any one of statements 43-72, wherein the disease or disorder is cancer.

Statement 78. The method of statement 77, wherein the disease or disorder is breast cancer.

Statement 79. The method according to any one of claims 43-72, wherein the disease or disorder is a bone disease or disorder.

Statement 80. The method of statement 79, wherein the disease or disorder is associated with bone resorption.

Statement 81. The method according to any one of statements 43-80 further comprising a second therapeutic agent.

Statement 82. The method according to any one of statements 43-81, wherein the compound is formulated in a pharmaceutical composition.

Statement 83. The method of statement 82, wherein the pharmaceutical composition further comprises an excipient.

Statement 84. The method of statement 82, wherein the pharmaceutical composition is formulated for administration orally, topically, intravenously, or via injection.

Statement 85. The method according to any one of statements 82-84, wherein the pharmaceutical composition is formulated as a unit dose.

Statement 86. The method according to any one of statements 43-85, wherein the patient is a mammal.

Statement 87. The method of statement 86, wherein the mammal is a human.

Statement 88. The method according to any one of statements 43-87, wherein the compound is administered once.

Statement 89. The method according to any one of statements 43-87, wherein the compound is administered two or more times.

Statement 90. The method of statement 89, wherein the compound is administered daily, weekly, or monthly.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synthesis of ERR Modulating Compounds

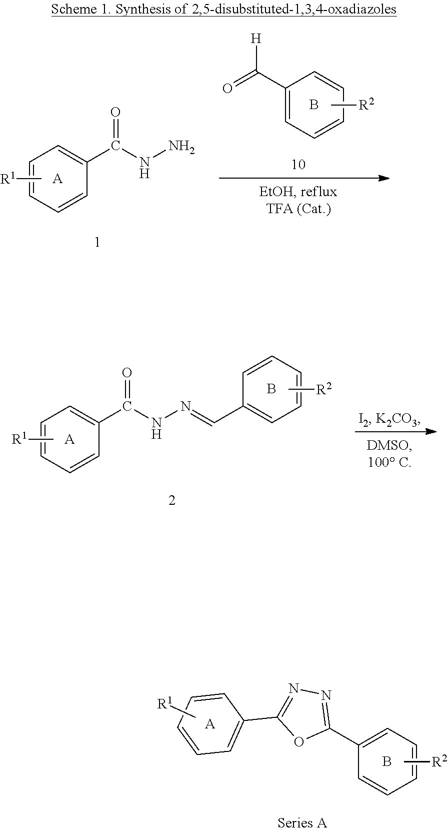

### General Procedure for the Synthesis of 2,5-Disubstituted-1,3,4-oxadiazoles (Series A)

### Step 1: Synthesis of arylidene benzohydrazides 2

To the solution of acid hydrazide (1 equiv) in anhydrous ethanol (5 mL), an equimolar of appropriate aldehydes (1 equiv) and a few drops of trifluoro acetic acid were added. The solution was heated under reflux and monitored by TLC till completion. The solution was then cooled to room temperature and the precipitate obtained that was filtered off and crystallized from ethanol.

### Step 2: Synthesis of 2,5-disubstituted-1,3,4-oxadiazoles (Series A)

Arylidene benzohydrazide (1.0 mmol) was dissolved in DMSO (5 mL), and potassium carbonate (3 mmol) and iodine (1.2 mmol) were added in order (Yu et al., 2013). The reaction mixture was heated at 100° C. with continuous stirring and monitored by TLC till completion. The mixture was then cooled to room temperature, treated with 5% sodium thiosulfate (20 mL), extracted with ethyl acetate (10 mL×3). The combined organic layer was washed with brine (10 mL×1), dried over anhydrous magnesium sulfate, and evaporated in vacuo. The obtained residue was purified with flash column chromatography using a mixture of ethyl acetate and hexanes.

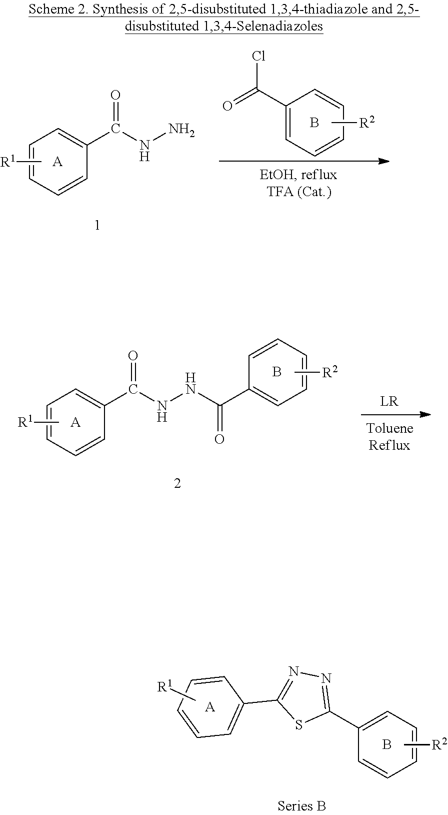

### General Procedure for the Synthesis of 2,5-Disubstituted 1,3,4-thiadiazole (Series B)

### Step 1: Synthesis of 1,2-diacylhydrazines

A suspension of acid hydrazide (5.0 mmol) and anhydrous sodium carbonate (5.0 mmol) in dry tetrahydrofuran (30 mL) and water (30 mL) was added to a stirring solution of the corresponding acid chloride (5.5 mmol) in 15 mL of tetrahydrofuran at 0° C. The mixture was stirred at 0° C. for 1 h, and at room temperature for 2 h. The formed precipitate was by filtered and washed with tetrahydrofuran and ethyl ether (10 mL×3) and dried in vacuo.

### Step 2: Synthesis of 2,5-disubstituted-1,3,4-oxadiazoles (Series B) (Pradham et al., 2016)

Lawesson's reagent (1.0 mmol) was added to a suspension of 1,2-diacylhydrazines (1.0 mmol) in 15 mL of dry toluene and the mixture was refluxed for 10 h. After completion, the reaction mixture was evaporated to dryness, the residue was dissolved in methylene chloride, and purified by flash chromatography using ethyl acetate/hexanes to afford pure compound.

TABLE 3
Characterization of Compounds

| | | | LC/MS | |
|---|---|---|---|---|
| | | | m/z | |
| Entry | IUPAC Name | Structure | [M + H⁺] | NMR |
| 7 | 2-(4- nitrophenyl)-5- phenyl-1,3,4- oxadiazole | 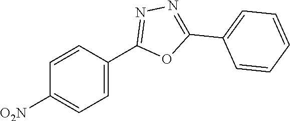 [SMILES: O=[N+]([O-])c1ccc(-c2nnc(-c3ccccc3)o2)cc1] | 268.1 | ¹H NMR (400 MHz, CDCl3) δ 8.45-8.38 (m, 2H), 8.38-8.29 (m, 2H), 8.22-8.11 (m, 2H), 7.66- 7.51 (m, 3H). ¹³C{1H} NMR (100 MHz, CDCl₃) δ 174.3, 165.8, 165.1, 162.5, 160.4, 133.7, 133.6, 132.2, 129.1, 127.1, 123.1, 120.6, 120.4, 114.9, 114.8, 113.0, 112.7. |
| 10 | 2-(2- nitrophenyl)-5- phenyl-1,3,4- oxadiazole | 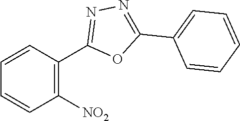 [SMILES: O=[N+]([O-])c1ccccc1-c1nnc(-c2ccccc2)o1] | 268.1 | ¹H NMR (400 MHz, CDCl₃) δ 8.15-8.01 (m, 4H), 7.84- 7.72 (m, 2H), 7.62-7.47 (m, 3H). ¹³C{1H} NMR (100 MHz, CDCl₃) δ 174.3, 165.7, 161.2, 148.2, 133.0, 132.4, 132.1, 131.6, 129.1, 127.1, 124.6, 123.3, 118.6. |
| 11 | 2-(2- nitrophenyl)-5- (4-nitrophenyl)- 1,3,4-oxadiazole | 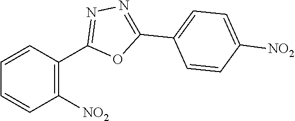 [SMILES: O=[N+]([O-])c1ccc(-c2nnc(-c3ccccc3[N+](=O)[O-])o2)cc1] | 313.1 | ¹H NMR (500 MHz, CDCl₃) δ 7.82-7.77 (m, 2H), 7.76- 7.72 (m, 2H), 7.32-7.24 (m, 4H). |
| 12 | 2-(3- nitrophenyl)-5- (4-nitrophenyl)- 1,3,4-oxadiazole | 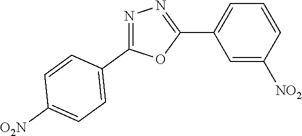 [SMILES: O=[N+]([O-])c1ccc(-c2nnc(-c3cccc([N+](=O)[O-])c3)o2)cc1] | 313.1 | ¹H NMR (500 MHz, CDCl₃) δ 8.29-8.23 (m, 1H), 8.22 8.15 (m, 2H), 8.05 (ddq, J = 6.2, 3.7, 1.2 Hz, 1H), 7.98 (ddt, J = 4.9, 2.7, 1.3 Hz, 1H), 7.95-7.89 (m, 1H), 7.28-7.22 (m, 2H). |
| 13 | 2,5-bis(4- nitrophenyl)- 1,3,4-oxadiazole | 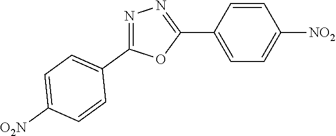 [SMILES: O=[N+]([O-])c1ccc(-c2nnc(-c3ccc([N+](=O)[O-])cc3)o2)cc1] | 313.0 | ¹H NMR (400 MHz, CDCl₃) δ 7.95 (dd, J = 8.9, 2.2 Hz, 2H), 7.87 (dt, J = 8.9, 2.5 Hz, 2H), 7.63 (dd, J = 8.8, 2.2 Hz, 2H), 7.01 (dd, J = 9.2, 2.2 Hz, 2H). |
| 14 | 2-phenyl-5- (quinolin-2-yl)- 1,3,4-oxadiazole | 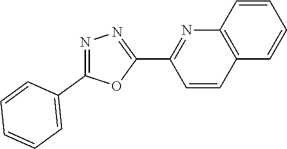 [SMILES: c1ccc(-c2nnc(-c3ccc4ccccc4n3)o2)cc1] | 274.1 | ¹H NMR (500 MHz, CDCl₃) δ 8.40-8.23 (m, 5H), 7.91- 7.76 (m, 2H), 7.63 (t, J = 7.5 Hz, 1H), 7.56 (t, J = 7.6 Hz, 3H). |
| 15 | 2-phenyl-5- (quinolin-4-yl)- 1,3,4-oxadiazole | 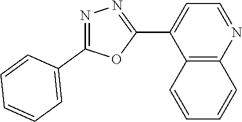 [SMILES: c1ccc(-c2nnc(-c3ccnc4ccccc34)o2)cc1] | 274.1 | ¹H NMR (CDCl₃) δ 9.20 (d, J = 8.5 Hz, 1H), 9.03 (d, J = 4.5 Hz, 1H), 8.16-8.14 (m, 3H), 7.96 (d, J = 4.5 Hz, 1H), 7.77-7.74 (m, 1H), 7.68-7.65 (m, 1H), 7.52-7.49 (m, 3H). |
| 16 | 2-(2- nitrobenzyl)-5- (4- (trifluoromethyl) phenyl)-1,3,4- oxadiazole | 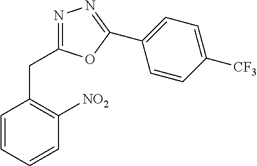 [SMILES: O=[N+]([O-])c1ccccc1Cc1nnc(-c2ccc(C(F)(F)F)cc2)o1] | 350.1 | ¹H NMR (CDCl₃) δ 8.34-8.30 (m, 3H), 7.92-7.88 (m, 2H), 7.54-7.51 (m, 3H), 3.77 (s, 2H). |
| 17 | 2-(4- methoxyphenyl)- 5-phenyl-1,3,4- oxadiazole | 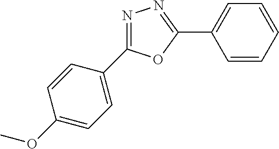 [SMILES: COc1ccc(-c2nnc(-c3ccccc3)o2)cc1] | 253.1 | ¹H NMR (500 MHz, CDCl₃) δ 8.08-8.00 (m, 2H), 7.08 (d, J = 3.4 Hz, 1H), 7.04- 6.98 (m, 2H), 6.21 (d, J = 3.4 Hz, 1H), 3.88 (d, J = 2.2 Hz, 3H), 2.44 (s, 3H). |
| 18 | 2-(furan-2-yl)-5- (4- methoxyphenyl)- 1,3,4-oxadiazole | 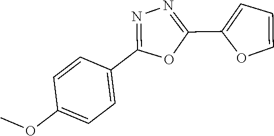 [SMILES: COc1ccc(-c2nnc(-c3ccco3)o2)cc1] | 243.1 | ¹H NMR (400 MHz, CDCl₃) δ 8.07 (dq, J = 8.9, 1.9, 1.4 Hz, 2H), 7.67 (dd, J = 1.9, 0.9 Hz, 1H), 7.23-7.20 (m, 1H), 7.05-7.00 (m, 2H), 6.63 (dt, J = 3.0, 1.4 Hz, 1H), 3.90 (d, J = 1.0 Hz, 3H). |
| 19 | 2-(4- methoxyphenyl)- 5-(5- methylfuran-2- yl)-1,3,4- oxadiazole | 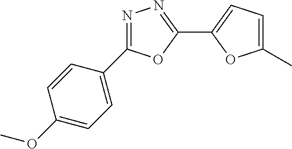 | 257.0 | ¹H NMR (500 MHz, CDCl₃) δ 8.08-8.00 (m, 2H), 7.08 (d, J = 3.4 Hz, 1H), 7.04- 6.98 (m, 2H), 6.21 (d, J = 3.4 Hz, 1H), 3.88 (d, J = 2.2 Hz, 3H), 2.44 (s, 3H). |
| 20 | 2-(4- methoxyphenyl)- 5-(thiophen-2- yl)-1,3,4- oxadiazole | 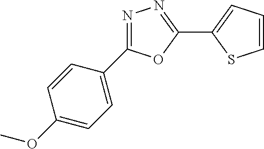 [SMILES: COc1ccc(-c2nnc(-c3cccs3)o2)cc1] | 259.1 | ¹H NMR (400 MHz, CDCl₃) δ 8.10-8.02 (m, 2H), 7.82 (dt, J = 3.8, 1.0 Hz, 1H), 7.57 (dt, J = 5.0, 1.1 Hz, 1H), 7.20 (ddd, J = 4.5, 3.6, 0.8 Hz, CH), 7.06-7.00 (m, 2H), 3.90 (d, J = 0.8 Hz, 3H). |
| 21 | 4-(5-(4- methoxyphenyl)- 1,3,4-oxadiazol- 2-yl)-N,N- dimethylaniline | 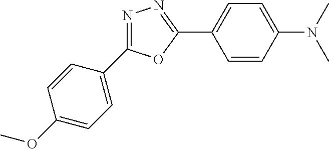 | 296.1 | ¹H NMR (500 MHz, CDCl₃) δ 8.09-8.04 (m, 2H), 7.98 (ddq, J = 9.2, 4.4, 2.6 Hz, 2H), 7.04 (dq, J = 9.0, 2.5 Hz, 2H), 6.78 (dq, J = 9.3, 2.5, 2.1 Hz, 2H), 3.91-3.89 (m, 3H), 3.09-3.06 (m, 6H). ¹³C NMR (101 MHz, CDCl₃) δ 164.85, 163.49, 161.95, 152.23, 128.45, 128.22, 116.96, 114.39, 111.60, 111.13, 55.47, 40.15. |
| 22 | 2-(4- methoxyphenyl)- 5-(quinolin-4-yl)- 1,3,4-oxadiazole | 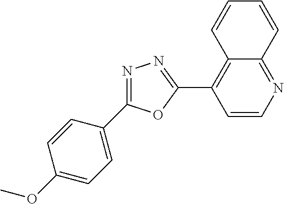 [SMILES: COc1ccc(-c2nnc(-c3ccnc4ccccc34)o2)cc1] | 304.1 | ¹H NMR (500 MHz, CDCl₃) δ 9.31 (d, J = 8.5 Hz, 1H), 9.10 (d, J = 4.5 Hz, 1H), 8.23 (d, J = 8.4 Hz, 1H), 8.16 (d, J = 8.5 Hz, 2H), 8.08 (d, J = 4.4 Hz, 1H), 7.85 (ddd, J = 8.4, 6.9, 1.5 Hz, 1H), 7.79- 7.73 (m, 1H), 7.08 (d, J = 8.6 Hz, 2H), 3.92 (s, 3H). |
| 23 | 2-(4- methoxyphenyl)- 5-(quinolin-2-yl)- 1,3,4-oxadiazole | 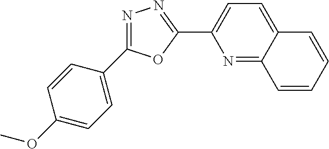 [SMILES: COc1ccc(-c2nnc(-c3ccc4ccccc4n3)o2)cc1] | 304.0 | ¹H NMR (500 MHz, CDCl₃) δ 8.40-8.31 (m, 2H), 8.29 (dd, J = 9.1, 5.3 Hz, 1H), 8.22 (td, J = 6.4, 2.6 Hz, 2H), 7.92-7.87 (m, 1H), 7.85- 7.78 (m, 1H), 7.68-7.61 (m, 1H), 7.06 (dt, J = 7.9, 3.8 Hz, 2H), 3.90 (d, J = 5.8 Hz, 3H). |
| 24 | 2-(4- methoxyphenyl)- 5-(pyridin-2-yl)- 1,3,4-oxadiazole | 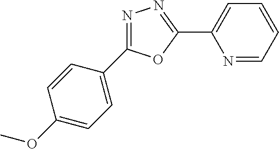 [SMILES: COc1ccc(-c2nnc(-c3ccccn3)o2)cc1] | 254.1 | ¹H NMR (500 MHz, CDCl₃) δ 8.81 (ddt, J = 4.8, 1.8, 1.1 Hz, 1H), 8.31 (ddq, J = 7.9, 2.2, 1.0 Hz, 1H), 8.18-8.12 (m, 2H), 7.91 (tq, J = 7.6, 1.6 Hz, 1H), 7.47 (ddt, J = 7.6, 4.9, 1.2 Hz, 1H), 7.06-6.99 (m, 2H), 3.91-3.88 (m, 3H). |
| 25 | 2-(4- methoxyphenyl)- 5-(2- nitrophenyl)- 1,3,4-oxadiazole | 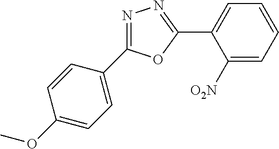 [SMILES: COc1ccc(-c2nnc(-c3ccccc3[N+](=O)[O-])o2)cc1] | 298.1 | ¹H NMR (400 MHz, CDCl₃) δ 8.09 (dt, J = 7.5, 1.3 Hz, 1H), 8.06-7.95 (m, 3H), 7.84-7.70 (m, 2H), 7.06- 6.97 (m, 2H), 3.89 (s, 3H). ¹³C {1H} NMR (100 MHz, CDCl₃) δ 174.3, 165.7, 162.6, 160.7, 133.0, 132.3, 131.5, 128.9, 124.6, 118.7, 115.7, 114.6, 55.5. |
| 26 | 2-(4- methoxyphenyl)- 5-(3- nitrophenyl)- 1,3,4-oxadiazole | 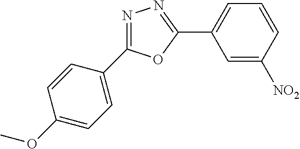 [SMILES: COc1ccc(-c2nnc(-c3cccc([N+](=O)[O-])c3)o2)cc1] | 298.0 | ¹H NMR (CDCl₃) δ 8.59 (s, 1H), 8.36-8.33 (m, 1H), 8.22-8.11 (m, 2H), 8.01 (d, J = 7.5 Hz, 2H), 7.22 (d, J = 7.5 Hz, 2H), 3.85 (s, 3H). |
| 27 | 2-(4- methoxyphenyl)- 5-(4- nitrophenyl)- 1,3,4-oxadiazole | 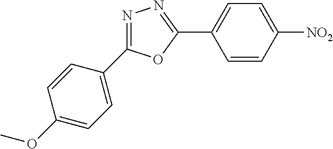 [SMILES: COc1ccc(-c2nnc(-c3ccc([N+](=O)[O-])cc3)o2)cc1] | 298.1 | ¹H NMR (CDCl₃) 8.21 (d, J = 151.9 Hz, 6H), 7.02 (s, 2H), 3.94 (d, J = 69.7 Hz, 3H). |
| 28 | 2-(2- chlorophenyl)-5- (4- methoxyphenyl)- 1,3,4-oxadiazole | 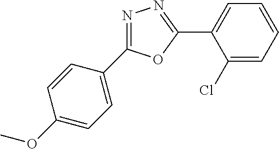 [SMILES: COc1ccc(-c2nnc(-c3ccccc3Cl)o2)cc1] | 287.1 | ¹H NMR (500 MHz, CDCl₃) δ 8.13-8.05 (m, 3H), 7.58 (dt, J = 8.1, 1.2 Hz, 1H), 7.51- 7.40 (m, 2H), 7.09-7.01 (m, 2H), 3.90 (d, J = 1.0 Hz, 3H). |
| 29 | 2-(3- methoxyphenyl)- 5-(4- methoxyphenyl)- 1,3,4-oxadiazole | 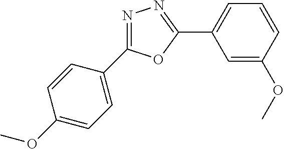 [SMILES: COc1ccc(-c2nnc(-c3cccc(OC)c3)o2)cc1] | 283.1 | ¹H NMR (500 MHz, CDCl₃) δ 8.11-8.05 (m, 2H), 7.70 (ddt, J = 7.6, 2.3, 1.0 Hz, 1H), 7.67-7.65 (m, 1H), 7.44 (td, J = 8.0, 1.5 Hz, 1H), 7.09 (ddt, J = 8.3, 3.5, 1.0 Hz, 1H), 7.06-7.02 (m, 2H), 3.92 (d, J = 1.5 Hz, 3H), 3.90 (d, J = 1.5 Hz, 3H). |
| 30 | 2,5-bis(4- methoxyphenyl)- 1,3,4-oxadiazole | 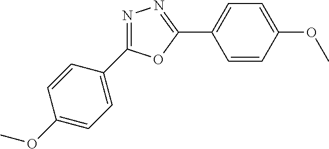 | 283.1 | ¹H NMR (500 MHz, CDCl₃). 8 8.08-8.04 (m, 4H), 7.04-7.02 (m, 4H), 3.89 (d, J = 0.8 Hz, 6H). |
| 4 | 2-phenyl-5-(p- tolyl)-1,3,4- oxadiazole | 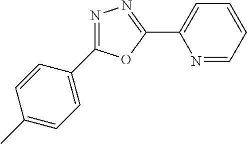 [SMILES: Cc1ccc(-c2nnc(-c3ccccn3)o2)cc1] | 237.1 | ¹H NMR (500 MHz, CDCl₃) δ 8.16 (dq, J = 6.1, 1.7 Hz, 2H), 8.07-8.01 (m, 2H), 7.55 (d, J = 6.1 Hz, 3H), 7.35 (d, J = 7.7 Hz, 2H), 2.46 (s, 3H). |
| 31 | 2-(furan-2-yl)-5- (p-tolyl)-1,3,4- oxadiazole | 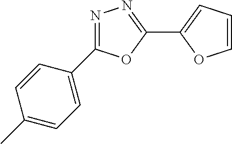 [SMILES: Cc1ccc(-c2nnc(-c3ccco3)o2)cc1] | 227.0 | ¹H NMR (500 MHz, CDCl₃) δ 8.02 (d, J = 8.0 Hz, 2H), 7.68 (d, J = 1.8 Hz, 1H), 7.34 (d, J = 7.9 Hz, 2H), 7.23 (d, J = 3.5 Hz, 1H), 6.63 (dd, J = 3.5, 1.7 Hz, 1H), 2.45 (s, 3H). |
| 32 | 2-(5- methylfuran-2- yl)-5-(p-tolyl)- 1,3,4-oxadiazole | 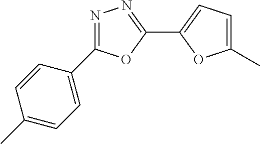 | 241.1 | ¹H NMR (500 MHz, CDCl₃) δ 8.04-7.97 (m, 2H), 7.32 (d, J = 7.9 Hz, 2H), 7.10 (d, J = 3.4 Hz, 1H), 6.22 (d, J = 3.4 Hz, 1H), 2.45 (s, 3H), 2.43 (s, 3H). |
| 33 | 2-(thiophen-2- yl)-5-(p-tolyl)- 1,3,4-oxadiazole | 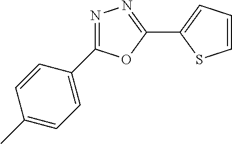 [SMILES: Cc1ccc(-c2nnc(-c3cccs3)o2)cc1] | 243.1 | ¹H NMR (400 MHz, CDCl₃) δ 8.03-7.99 (m, 2H), 7.84 (dd, J = 3.7, 1.2 Hz, 1H), 7.57 (dt, J = 4.9, 1.0 Hz, 1H), 7.35-7.32 (m, 2H), 7.20 (ddd, J = 4.4, 3.6, 0.7 Hz, 1H), 2.45 (s, 3H). |
| 34 | 2-(quinolin-4-yl)- 5-(p-tolyl)-1,3,4- oxadiazole | 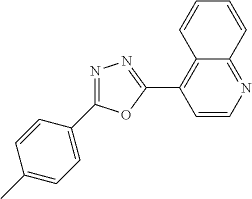 [SMILES: Cc1ccc(-c2nnc(-c3ccnc4ccccc34)o2)cc1] | 288.1 | ¹H NMR (400 MHz, CDCl₃) δ 9.05 (dd, J = 4.4, 1.5 Hz, 1H), 8.89 (dd, J = 8.7, 1.5 Hz, 1H), 8.23 (dd, J = 8.4, 1.5 Hz, 1H), 7.97 (dd, J = 8.2, 1.6 Hz, 2H), 7.84 (ddt, J = 8.4, 6.8, 1.5 Hz, 1H), 7.76- 7.68 (m, 2H), 7.37-7.31 (m, 2H), 2.46 (s, 3H). |
| 35 | 2-(quinolin-2-yl)- 5-(p-tolyl)-1,3,4- oxadiazole | 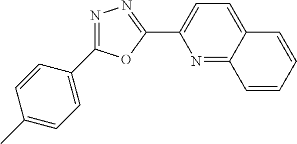 [SMILES: Cc1ccc(-c2nnc(-c3ccc4ccccc4n3)o2)cc1] | 288.1 | ¹H NMR (500 MHz, CDCl₃). 8 8.41-8.33 (m, 2H), 8.32-8.26 (m, 1H), 8.19-8.16 (m, 2H), 7.90 (dd, J = 8.1, 1.4 Hz, 1H), 7.82 (ddd, J = 8.4, 6.9, 1.5 Hz, 1H), 7.65 (ddd, J = 8.1, 6.8, 1.2 Hz, 1H), 7.36 (d, J = 7.9 Hz, 2H), 2.46 (s, 3H). |
| 36 | 2-(pyridin-2-yl)- 5-(p-tolyl)-1,3,4- oxadiazole | 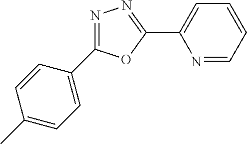 [SMILES: Cc1ccc(-c2nnc(-c3ccccn3)o2)cc1] | 238.1 | ¹H NMR (500 MHz, CDCl₃) δ 8.64 (d, J = 4.7 Hz, 1H), 8.11 (d, J = 8.0 Hz, 1H), 7.91 (d, J = 7.8 Hz, 2H), 7.77- 7.69 (m, 1H), 7.30 (dd, J = 7.8, 4.8 Hz, 1H), 7.14 (d, J = 7.9 Hz, 2H), 2.23 (s, 3H). |
| 37 | 2-(2- nitrophenyl)-5- (p-tolyl)-1,3,4- oxadiazole | 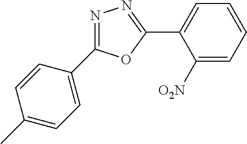 [SMILES: Cc1ccc(-c2nnc(-c3ccccc3[N+](=O)[O-])o2)cc1] | 282.1 | ¹H NMR (400 MHz, CDCl₃) δ 8.10 (dt, J = 7.6, 1.4 Hz, 1H), 8.05 (dt, J = 7.9, 1.3 Hz, 1H), 7.98-7.94 (m, 2H), 7.78 (dtt, J = 22.0, 7.6, 1.4 Hz, 2H), 7.36-7.31 (m, 2H), 2.45 (s, 3H). |
| 38 | 2-(3- nitrophenyl)-5- (p-tolyl)-1,3,4- oxadiazole | 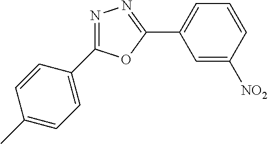 [SMILES: Cc1ccc(-c2nnc(-c3cccc([N+](=O)[O-])c3)o2)cc1] | 282.1 | ¹H NMR (400 MHz, CDCl₃) δ 8.40-8.34 (m, 1H), 7.97- 7.93 (m, 2H), 7.58-7.53 (m, 1H), 7.47-7.41 (m, 2H), 7.34-7.28 (m, 2H), 2.44 (s, 3H). |
| 39 | 2-(4- nitrophenyl)-5- (p-tolyl)-1,3,4- oxadiazole | 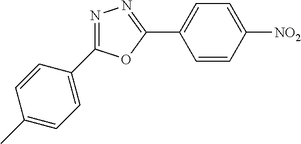 [SMILES: Cc1ccc(-c2nnc(-c3ccc([N+](=O)[O-])cc3)o2)cc1] | 282.0 | ¹H NMR (500 MHz, CDCl₃) δ 8.44-8.39 (m, 2H), 8.35 (d, J = 8.9 Hz, 2H), 8.06 (d, J = 8.0 Hz, 2H), 7.38 (d, J = 7.9 Hz, 2H), 2.48 (s, 3H). |
| 40 | 2-(2- chlorophenyl)-5- (p-tolyl)-1,3,4- oxadiazole | 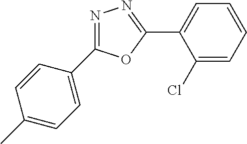 [SMILES: Cc1ccc(-c2nnc(-c3ccccc3Cl)o2)cc1] | 271.1 | ¹H NMR (400 MHz, CDCl₃) δ 8.12 (dt, J = 7.5, 1.4 Hz, 1H), 8.08-8.03 (m, 2H), 7.60 (dt, J = 8.0, 1.2 Hz, 1H), 7.50 (tdd, J = 8.0, 1.9, 0.8 Hz, 1H), 7.45 (tt, J = 7.5, 1.2 Hz, 1H), 7.38-7.33 (m, 2H), 2.47 (s, 3H). |
| 41 | 2-(3- methoxyphenyl)- 5-(p-tolyl)-1,3,4- oxadiazole | 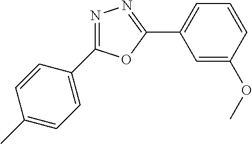 [SMILES: COc1cccc(-c2nnc(-c3ccc(C)cc3)o2)c1] | 267.1 | ¹H NMR (400 MHz, CDCl₃) δ 8.06-8.01 (m, 2H), 7.74- 7.70 (m, 1H), 7.70-7.66 (m, 1H), 7.48-7.42 (m, 1H), 7.37-7.32 (m, 2H), 7.13- 7.07 (m, 1H), 3.93-3.91 (m, 3H), 2.45 (s, 3H). |
| 42 | 2-(4- methoxyphenyl)- 5-(p-tolyl)-1,3,4- oxadiazole | 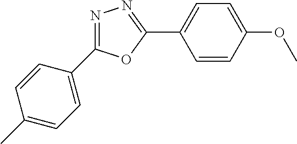 | 267.1 | ¹H NMR (400 MHz, CDCl₃) δ 8.12-8.07 (m, 2H), 8.03 (dd, J = 8.3, 2.0 Hz, 2H), 7.35 (ddt, J = 7.9, 1.4, 0.8 Hz, 2H), 7.07-7.03 (m, 2H), 3.91 (d, J = 2.3 Hz, 3H), 2.46 (d, J = 2.0 Hz, 3H) |
| 43 | 2-phenyl-5-(4- (trifluoromethyl) phenyl)-1,3,4- oxadiazole | 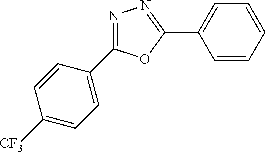 [SMILES: FC(F)(F)c1ccc(-c2nnc(-c3ccccc3)o2)cc1] | 291.1 | ¹H NMR (500 MHz, CDCl₃) δ 8.29 (d, J = 8.1 Hz, 2H), 8.18 (ddt, J = 8.2, 6.4, 1.6 Hz, 2H), 7.83 (t, J = 8.0 Hz, 2H), 7.63-7.54 (m, 3H). |
| 44 | 2-(furan-2-yl)-5- (4- (trifluoromethyl) phenyl)-1,3,4- oxadiazole | 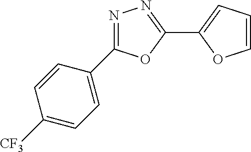 [SMILES: FC(F)(F)c1ccc(-c2nnc(-c3ccco3)o2)cc1] | 281.1 | ¹H NMR (400 MHz, CDCl₃) δ 8.31-8.24 (m, 2H), 7.85- 7.79 (m, 2H), 7.74-7.69 (m, 1H), 7.31 (dt, J = 8.6, 4.2 Hz, 1H), 6.67 (ddp, J = 5.8, 4.1, 1.9 Hz, 1H). ¹³C NMR (126 MHz, CDCl₃) δ 162.79, 157.95, 146.11, 139.12, 133.32, 127.34, 126.75, 126.24, 126.21, 126.18, 126.15, 122.47, 114.77, 112.41. |
| 45 | 2-(5- methylfuran-2- yl)-5-(4- (trifluoromethyl) phenyl)-1,3,4- oxadiazole | 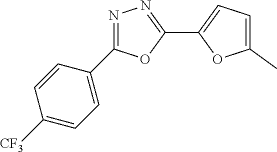 [SMILES: Cc1ccc(-c2nnc(-c3ccc(C(F)(F)F)cc3)o2)o1] | 295.1 | ¹H NMR (500 MHz, CDCl₃) δ 8.27 (d, J = 8.1 Hz, 2H), 7.86-7.73 (m, 2H), 7.18 (d, J = 2.8 Hz, 1H), 6.26 (s, 1H), 2.48 (d, J = 2.0 Hz, 3H). ¹³C NMR (126 MHz, CDCl₃) 8 162.44, 158.08, 157.04, 137.41, 127.25, 126.14, 126.11, 116.04, 108.77, 13.89. |
| 46 | 2-(thiophen-2- yl)-5-(4- (trifluoromethyl) phenyl)-1,3,4- oxadiazole | 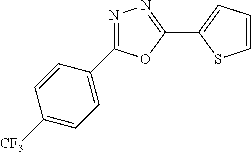 [SMILES: FC(F)(F)c1ccc(-c2nnc(-c3cccs3)o2)cc1] | 297.1 | ¹H NMR (500 MHz, CDCl₃) δ 8.27 (dd, J = 8.4, 2.3 Hz, 2H), 7.89 (dt, J = 3.3, 1.6 Hz, 1H), 7.82 (dd, J = 8.4, 2.3 Hz, 2H), 7.63 (dq, J = 4.7, 1.3 Hz, 1H), ¹³C NMR (126 MHz, CDCl₃) δ 130.73, 130.69, 130.24, 128.37, 128.33, 127.30, 127.26, 126.16, 118.01, 77.28, 77.02, 76.77. |
| 47 | 2-(quinolin-4-yl)- 5-(4- (trifluoromethyl) phenyl)-1,3,4- oxadiazole | 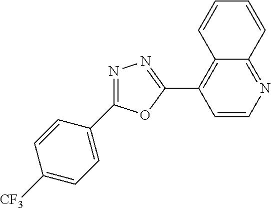 [SMILES: FC(F)(F)c1ccc(-c2nnc(-c3ccnc4ccccc34)o2)cc1] | 342.1 | ¹H NMR (400 MHz, CDCl₃) δ 9.30 (dd, J = 8.6, 3.7 Hz, 1H), 9.17-9.11 (m, 1H), 8.36 (dd, J = 8.4, 3.9 Hz, 2H), 8.27 (dd, J = 8.6, 3.9 Hz, 1H), 8.15-8.10 (m, 1H), 7.89 (dddd, J = 10.0, 8.4, 3.4, 1.5 Hz, 3H), 7.80 (tdd, J = 6.9, 3.7, 1.9 Hz, 1H). ¹³C NMR (101 MHz, CDCl₃) δ 163.75, 163.48, 149.58, 149.00, 130.49, 130.25, 128.92, 127.75, 127.60, 126.58, 126.38, 126.35, 126.32, 126.29, 126.12, 123.83, 120.57. |
| 48 | 2-(quinolin-2-yl)- 5-(4- (trifluoromethyl) phenyl)-1,3,4- oxadiazole | 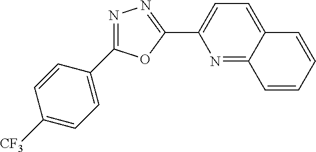 [SMILES: FC(F)(F)c1ccc(-c2nnc(-c3ccc4ccccc4n3)o2)cc1] | 342.1 | ¹H NMR (400 MHz, CDCl₃) δ 8.40 (dtd, J = 16.2, 7.9, 3.4 Hz, 4H), 8.30 (dd, J = 8.6, 3.7 Hz, 1H), 7.95-7.90 (m, 1H), 7.85 (dq, J = 7.7, 5.4, 3.7 Hz, 3H), 7.69 (td, J = 7.7, 3.5 Hz, 1H). ¹³C NMR (101 MHz, CDCl₃) δ 164.66, 147.98, 143.07, 137.56, 133.76, 133.49, 130.69, 130.11, 128.80, 128.49, 127.84, 127.77, 126.86, 126.15, 126.12, 126.09, 126.06, 124.66, 122.49, 119.90, 1.02. |
| 49 | 2-(pyridin-2-yl)- 5-(4- (trifluoromethyl) phenyl)-1,3,4- oxadiazole | 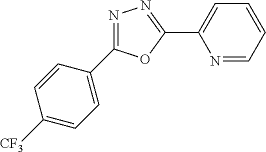 [SMILES: FC(F)(F)c1ccc(-c2nnc(-c3ccccn3)o2)cc1] | 292.1 | ¹H NMR (400 MHz, CDCl₃) δ 8.86-8.79 (m, 1H), 8.39- 8.31 (m, 3H), 7.96-7.90 (m, 1H), 7.80 (d, J = 7.8 Hz, 2H), 7.50 (ddd, J = 7.7, 4.8, 1.2 Hz, 1H). |
| 50 | 2-(2- nitrophenyl)-5- (4- (trifluoromethyl) phenyl)-1,3,4- oxadiazole | 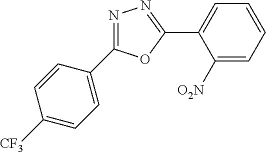 [SMILES: O=[N+]([O-])c1ccccc1-c1nnc(-c2ccc(C(F)(F)F)cc2)o1] | 336.0 | ¹H NMR (400 MHz, CDCl₃) δ 8.22 (t, J = 7.4 Hz, 2H), 8.14-8.07 (m, 2H), 7.88- 7.78 (m, 4H). ¹³C NMR (126 MHz, CDCl₃) δ 164.57, 161.88, 133.62, 133.25, 132.84, 131.81, 127.49, 126.58, 126.25, 126.22, 124.82, 124.58, 118.38 |
| 51 | 2-(3- nitrophenyl)-5- (4- (trifluoromethyl) phenyl)-1,3,4- oxadiazole | 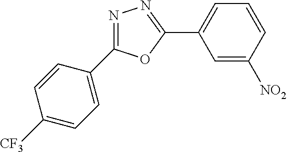 [SMILES: O=[N+]([O-])c1cccc(-c2nnc(-c3ccc(C(F)(F)F)cc3)o2)c1] | 336.0 | ¹H NMR (400 MHz, CDCl₃) δ 9.01-8.96 (m, 1H), 8.55 (dt, J = 7.6, 1.3 Hz, 1H), 8.46 (ddt, J = 8.2, 2.2, 1.1 Hz, 1H), 8.33 (d, J = 8.2 Hz, 2H), 7.85 (d, J = 8.2 Hz, 2H), 7.81 (t, J = 8.0 Hz, 1H). ¹³C NMR (101 MHz, CDCl₃) δ 164.16, 163.25, 148.71, 133.70, 132.60, 130.60, 127.48, 126.58, 126.47, 126.35, 126.32, 126.29, 126.26, 125.23, 121.87. |
| 52 | 2-(4- nitrophenyl)-5- (4- (trifluoromethyl) phenyl)-1,3,4- oxadiazole | 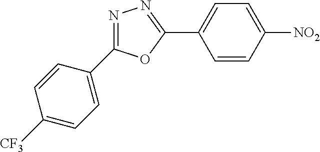 [SMILES: O=[N+]([O-])c1ccc(-c2nnc(-c3ccc(C(F)(F)F)cc3)o2)cc1] | 336.1 | ¹H NMR (500 MHz, CDCl₃) δ 8.45 (d, J = 8.0 Hz, 2H), 8.38 (d, J = 7.8 Hz, 2H), 8.32 (d, J = 7.9 Hz, 2H), 7.86 (d, J = 8.0 Hz, 2H). |
| 53 | 2-(2- chlorophenyl)-5- (4- (trifluoromethyl) phenyl)-1,3,4- oxadiazole | 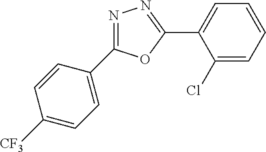 [SMILES: FC(F)(F)c1ccc(-c2nnc(-c3ccccc3Cl)o2)cc1] | 325.1 | |
| 54 | 2-(3- methoxyphenyl)- 5-(4- (trifluoromethyl) phenyl)-1,3,4- oxadiazole | 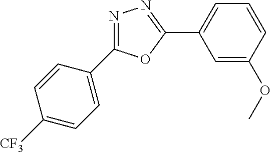 [SMILES: COc1cccc(-c2nnc(-c3ccc(C(F)(F)F)cc3)o2)c1] | 321.0 | ¹H NMR (500 MHz, CDCl₃) δ 8.29 (dd, J = 8.6, 2.1 Hz, 2H), 7.82 (dd, J = 8.5, 2.2 Hz, 2H), 7.74 (dp, J = 7.7, 1.3 Hz, 1H), 7.69 (td, J = 2.6, 1.4 Hz, 1H), 7.47 (td, J = 8.1, 2.3 Hz, 1H), 7.14 (ddt, J = 8.4, 2.7, 1.4 Hz, 1H), 3.93 (d, J = 2.3 Hz, 3H). |
| 55 | 2-(4- methoxyphenyl)- 5-(4- (trifluoromethyl) phenyl)-1,3,4- oxadiazole | 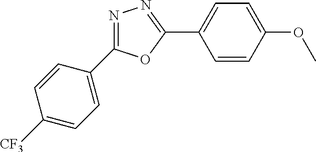 [SMILES: COc1ccc(-c2nnc(-c3ccc(C(F)(F)F)cc3)o2)cc1] | 321.1 | ¹H NMR (500 MHz, CDCl₃) δ 8.22 (td, J = 5.6, 2.4 Hz, 2H), 8.05 (ddd, J = 9.0, 3.5, 1.9 Hz, 2H), 7.77 (td, J = 5.5, 2.3 Hz, 2H), 7.02 (ddd, J = 9.0, 4.4, 1.9 Hz, 2H), 3.89- 3.86 (m, 3H). ¹³C NMR (126 MHz, CDCl₃) δ 165.06, 162.91, 162.60, 133.44, 133.18, 132.92, 128.81, 127.29, 127.28, 127.06, 126.87, 126.10, 126.07, 126.04, 126.01, 124.70, 122.53, 115.95, 114.58, 55.45. |
| 56 | 2-phenyl-5-(3- (trifluoromethyl) phenyl)-1,3,4- oxadiazole | 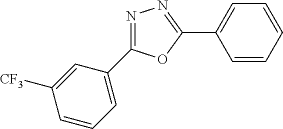 [SMILES: FC(F)(F)c1cccc(-c2nnc(-c3ccccc3)o2)c1] | 291.1 | ¹H NMR (500 MHz, CDCl₃) δ 8.39 (td, J = 11.5, 7.7 Hz, 2H), 8.22-8.14 (m, 2H), 7.85 (dd, J = 11.5, 7.4 Hz, 1H), 7.72 (dt, J = 11.4, 7.8 Hz, 1H), 7.58 (dtt, J = 9.4, 6.5, 3.1 Hz, 3H). |
| 57 | 2-(furan-2-yl)-5- (3- (trifluoromethyl) phenyl)-1,3,4- oxadiazole | 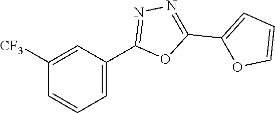 [SMILES: FC(F)(F)c1cccc(-c2nnc(-c3ccco3)o2)c1] | 281.1 | ¹H NMR (400 MHz, CDCl₃) δ 8.41-8.38 (m, 1H), 8.35 (dd, J = 7.8, 1.5 Hz, 1H), 7.85-7.81 (m, 1H), 7.72- 7.67 (m, 2H), 7.32-7.29 (m, 1H), 6.66 (dd, J = 3.5, 1.7 Hz, 1H). |
| 58 | 2-(thiophen-2- yl)-5-(3- (trifluoromethyl) phenyl)-1,3,4- oxadiazole | 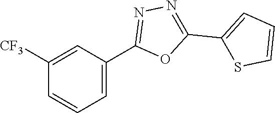 [SMILES: FC(F)(F)c1cccc(-c2nnc(-c3cccs3)o2)c1] | 297.1 | ¹H NMR (400 MHz, CDCl₃) δ 8.38 (s, 1H), 8.34 (dt, J = 8.1, 1.8 Hz, 1H), 7.90 (dt, J = 3.5, 1.7 Hz, 1H), 7.83 (d, J = 7.9 Hz, 1H), 7.71 (td, J = 7.9, 2.6 Hz, 1H), 7.63 (dt, J = 4.9, 1.6 Hz, 1H), 7.26-7.21 (m, 1H). ¹³C NMR (126 MHz, CDCl₃) δ 172.83, 162.83, 161.33, 131.97, 131.70, 130.65, 130.23, 130.05, 129.81, 128.31, 128.28, 128.25, 128.22, 124.80, 124.58, 123.78, 123.75, 123.72. |
| 59 | 2-(quinolin-4-yl)- 5-(3- (trifluoromethyl) phenyl)-1,3,4- oxadiazole | 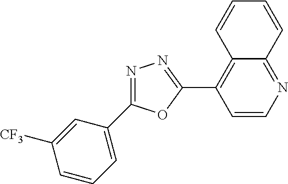 [SMILES: FC(F)(F)c1cccc(-c2nnc(-c3ccnc4ccccc34)o2)c1] | 342.1 | ¹H NMR (500 MHz, CDCl₃) δ 9.30 (dd, J = 8.6, 1.6 Hz, 1H), 9.14 (dd, J = 4.6, 1.5 Hz, 1H), 8.47 (d, J = 2.0 Hz, 1H), 8.44 (dd, J = 7.8, 1.6 Hz, 1H), 8.25 (dd, J = 8.5, 1.6 Hz, 1H), 8.13 (dd, J = 4.6, 1.5 Hz, 1H), 7.91-7.85 (m, 2H), 7.83-7.74 (m, 2H). |
| 60 | 2-(quinolin-2-yl)- 5-(3- (trifluoromethyl) phenyl)-1,3,4- oxadiazole | 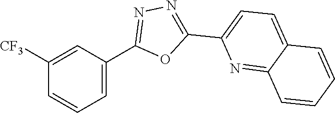 [SMILES: FC(F)(F)c1cccc(-c2nnc(-c3ccc4ccccc4n3)o2)c1] | 342.1 | ¹H NMR (500 MHz, CDCl₃) δ 8.59-8.53 (m, 1H), 8.50 (dd, J = 8.3, 4.1 Hz, 1H), 8.41 (qd, J = 8.6, 4.1 Hz, 2H), 8.32 (dd, J = 8.8, 4.2 Hz, 1H), 7.96-7.81 (m, 3H), 7.72 (ddd, J = 20.4, 9.8, 5.3 Hz, 2H). ¹³C NMR (101 MHz, CDCl₃) δ 164.67, 164.58, 147.98, 143.08, 137.63, 130.74, 130.58, 130.13, 129.81, 128.82, 128.64, 128.61, 128.53, 127.86, 124.46, 124.37, 124.34, 119.92. |
| 61 | 2-(pyridin-2-yl)- 5-(3- (trifluoromethyl) phenyl)-1,3,4- oxadiazole | 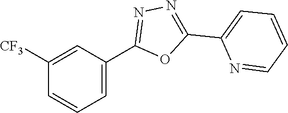 [SMILES: FC(F)(F)c1cccc(-c2nnc(-c3ccccn3)o2)c1] | 292.1 | ¹H NMR (400 MHz, CDCl₃) δ 8.86 (ddt, J = 4.8, 3.0, 1.4 Hz, 1H), 8.50 (d, J = 2.6 Hz, 1H), 8.45 (dt, J = 7.9, 1.8 Hz, 1H), 8.37 (ddt, J = 8.0, 2.2, 1.1 Hz, 1H), 7.96 (tq, J = 7.8, 1.7 Hz, 1H), 7.85 (d, J = 7.9 Hz, 1H), 7.71 (td, J = 7.8, 2.5 Hz, 1H), 7.54 (dddd, J = 7.8, 4.7, 3.1, 1.2 Hz, 1H). |
| 62 | 2-(2- nitrophenyl)-5- (3- (trifluoromethyl) phenyl)-1,3,4- oxadiazole | 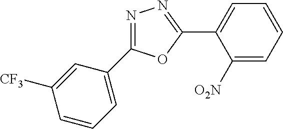 [SMILES: O=[N+]([O-])c1ccccc1-c1nnc(-c2cccc(C(F)(F)F)c2)o1] | 336.1 | ¹H NMR (400 MHz, CDCl₃) δ 8.35 (d, J = 1.7 Hz, 1H), 8.27 (dd, J = 8.1, 1.6 Hz, 1H), 8.12 (dt, J = 7.9, 1.3 Hz, 1H), 8.08 (dt, J = 7.3, 1.3 Hz, 1H), 7.87-7.79 (m, 3H), 7.70 (t, J = 7.8 Hz, 1H). |
| 63 | 2-(3- nitrophenyl)-5- (3- (trifluoromethyl) phenyl)-1,3,4- oxadiazole | 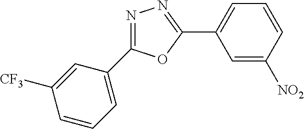 [SMILES: O=[N+]([O-])c1cccc(-c2nnc(-c3cccc(C(F)(F)F)c3)o2)c1] | 336.0 | ¹H NMR (400 MHz, CDCl₃) δ 8.99 (q, J = 2.0 Hz, 1H), 8.55 (dt, J = 7.8, 1.5 Hz, 1H), 8.49-8.42 (m, 2H), 8.40 (d, J = 7.9 Hz, 1H), 7.88 (d, J = 7.9 Hz, 1H), 7.81 (td, J = 8.0, 1.8 Hz, 1H), 7.75 (t, J = 7.9 Hz, 1H). |
| 64 | 2-(4- nitrophenyl)-5- (3- (trifluoromethyl) phenyl)-1,3,4- oxadiazole | 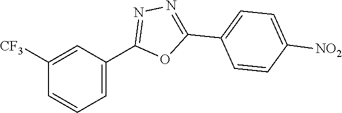 [SMILES: O=[N+]([O-])c1ccc(-c2nnc(-c3cccc(C(F)(F)F)c3)o2)cc1] | 336.0 | |
| 65 | 2-(2- chlorophenyl)-5- (3- (trifluoromethyl) phenyl)-1,3,4- oxadiazole | 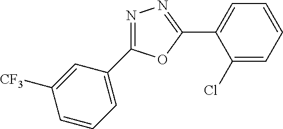 [SMILES: FC(F)(F)c1cccc(-c2nnc(-c3ccccc3Cl)o2)c1] | 325.0 | ¹H NMR (400 MHz, CDCl₃) δ 8.35 (d, J = 1.7 Hz, 1H), 8.27 (dd, J = 8.1, 1.6 Hz, 1H), 8.12 (dt, J = 7.9, 1.3 Hz, 1H), 8.08 (dt, J = 7.3, 1.3 Hz, 1H), 7.87-7.79 (m, 3H), 7.70 (t, J = 7.8 Hz, 1H). |
| 66 | 2-(3- methoxyphenyl)- 5-(3- (trifluoromethyl) phenyl)-1,3,4- oxadiazole | 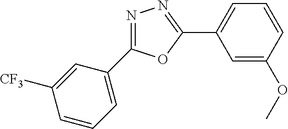 [SMILES: COc1cccc(-c2nnc(-c3cccc(C(F)(F)F)c3)o2)c1] | 321.1 | ¹H NMR (500 MHz, CDCl₃) δ 8.39 (s, 1H), 8.36 (d, J = 7.9 Hz, 1H), 7.83 (d, J = 7.9 Hz, 1H), 7.76-7.66 (m, 3H), 7.47 (td, J = 7.9, 2.4 Hz, 1H), 7.15-7.10 (m, 1H), 3.93 (d, J = 2.6 Hz, 3H). ¹³C NMR (101 MHz, CDCl₃) 8 165.00, 163.41, 160.05, 131.95, 131.68, 130.32, 130.06, 129.81, 128.26, 128.23, 128.20, 124.78, 124.65, 124.62, 123.78, 123.75, 123.72, 122.46, 119.44, 118.45, 111.77, 55.58. |
| 67 | 2-(4- methoxyphenyl)- 5-(4- (trifluoromethyl) phenyl)-1,3,4- oxadiazole | 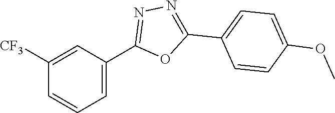 [SMILES: COc1ccc(-c2nnc(-c3cccc(C(F)(F)F)c3)o2)cc1] | 321.1 | ¹H NMR (500 MHz, CDCl₃) δ 8.38 (d, J = 2.1 Hz, 1H), 8.35 (d, J = 8.1 Hz, 1H), 8.13- 8.09 (m, 2H), 7.82 (d, J = 7.9 Hz, 1H), 7.72-7.67 (m, 1H), 7.08-7.03 (m, 2H), 3.92 (d, J = 2.1 Hz, 3H). ¹³C NMR (126 MHz, CDCl₃) 8 165.03, 162.95, 162.62, 129.95, 129.80, 129.78, 128.91, 128.89, 128.07, 124.94, 123.65, 123.61, 116.00, 114.63, 114.61, 55.55, 55.53. |
| 68 | 2-(5-phenyl- 1,3,4-oxadiazol- 2-yl)aniline | 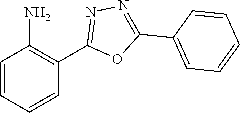 [SMILES: Nc1ccccc1-c1nnc(-c2ccccc2)o1] | 238.1 | ¹H NMR (400 MHz, CDCl₃) δ 8.23-8.09 (m, 2H), 7.86 (d, J = 7.9 Hz, 1H), 7.62- 7.48 (m, 3H), 7.32-7.25 (m, 1H), 6.88-6.74 (m, 2H), 5.91 (br s, 2H). ¹³C{1H} NMR (100 MHz, CDCl₃) 8 164.6, 162.8, 147.1, 132.5, 131.7, 129.1, 127.8, 126.9, 123.9, 116.8, 116.2, 105.8 |
| 69 | 3-(5-phenyl- 1,3,4-oxadiazol- 2-yl)aniline | 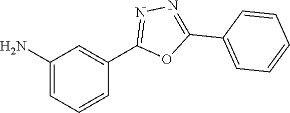 [SMILES: Nc1cccc(-c2nnc(-c3ccccc3)o2)c1] | 238.1 | ¹H NMR (400 MHz, CDCl₃) δ 8.20-8.06 (m, 2H), 7.62- 7.41 (m, 5H), 7.33-7.25 (m, 1H), 6.89-6.81 (m, 1H), 3.91 (br s, 2H). ¹³C{1H} NMR (100 MHz, CDCl₃) 8 164.8, 164.5, 147.0, 131.7, 130.1, 129.1, 126.9, 124.7, 124.0, 118.3, 117.0, 112.9 |
| 70 | 4-(5-phenyl- 1,3,4-oxadiazol- 2-yl)aniline | 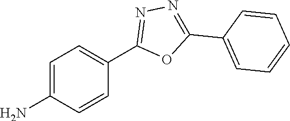 [SMILES: Nc1ccc(-c2nnc(-c3ccccc3)o2)cc1] | 238.1 | ¹H NMR (400 MHz, CDCl₃) δ 8.16-8.08 (m, 2H), 7.93 (d, J = 8.3 Hz, 2H), 7.57- 7.48 (m, 3H), 6.76 (d, J = 8.3 Hz, 2H), 4.08 (br s, 2H). ¹³C{1H} NMR (100 MHz, CDCl₃) δ 165.0, 163.7, 149.7, 131.3, 129.0, 128.7, 126.8, 124.3, 114.7, 113.6 |
| 71 | 5-methoxy-2-(5- phenyl-1,3,4- oxadiazol-2- yl)aniline | 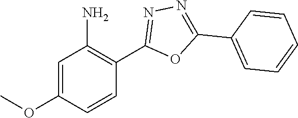 [SMILES: COc1ccc(-c2nnc(-c3ccccc3)o2)c(N)c1] | 268.1 | ¹H NMR (400 MHz, CDCl₃) δ 8.18-8.09 (m, 2H), 7.77 (dd, J = 8.8, 1.1 Hz, 1H), 7.58-7.48 (m, 3H), 6.41- 6.35 (m, 1H), 6.28 (d, J = 1.1 Hz, 1H), 5.95 (br s, 2H), 3.82 (s, 3H). ¹³C{1H} NMR (100 MHz, CDCl₃) δ 164.6, 163.2, 162.3, 149.0, 131.4, 129.4, 129.0, 126.8, 124.1, 105.0, 99.6, 99.5, 55.3 |
| 72 | 5-bromo-2-(5- phenyl-1,3,4- oxadiazol-2- yl)aniline | 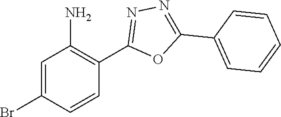 [SMILES: Nc1cc(Br)ccc1-c1nnc(-c2ccccc2)o1] | 317.1 | ¹H NMR (400 MHz, CDCl₃) δ 8.19-8.09 (m, 2H), 7.80 (dd, J = 8.0, 1.4 Hz, 1H), 7.64-7.49 (m, 3H), 7.41 (dd, J = 7.8, 1.5 Hz, 1H), 6.73 (t, J = 7.9 Hz, 1H), 6.45 (br s, 2H). ¹³C{1H} NMR (100 MHz, CDCl₃) δ 164.0, 163.1, 143.4, 132.2, 131.9, 129.1, 127.0, 126.4, 123.7, 120.1, 116.5, 106.8 |
| 73 | 4-bromo-2-(5- phenyl-1,3,4- oxadiazol-2- yl)aniline | 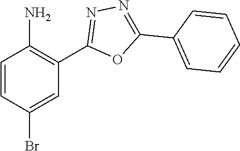 [SMILES: Nc1ccc(Br)cc1-c1nnc(-c2ccccc2)o1] | 317.1 | ¹H NMR (400 MHz, CDCl₃) δ 8.14 (dd, J = 7.6, 2.0 Hz, 2H), 7.94 (d, J = 2.2 Hz, 1H), 7.60-7.51 (m, 3H), 7.34 (dd, J = 8.8, 2.3 Hz, 1H), 6.71 (d, J = 8.8 Hz, 1H), 5.96 (br s, 2H). ¹³C{1H} NMR (100 MHz, CDCl₃) δ 163.5, 163.0, 146.0, 135.1, 131.9, 129.8, 129.1, 127.0, 123.6, 117.9 |
| 74 | 2-(5-(4- methoxyphenyl)- 1,3,4-oxadiazol- 2-yl)aniline | 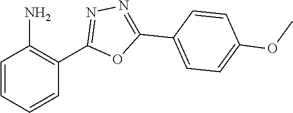 [SMILES: COc1ccc(-c2nnc(-c3ccccc3N)o2)cc1] | 268.1 | ¹H NMR (400 MHz, CDCl₃) δ 8.07 (d, J = 7.4 Hz, 2H), 7.84 (d, J = 7.9 Hz, 1H), 7.31- 7.24 (m, 1H), 7.03 (d, J = 7.3 Hz, 2H), 6.85-6.75 (m, 2H), 5.91 (s, 2H), 3.89 (s, 3H). ¹³C{1H} NMR (100 MHz, CDCl₃) δ 164.2, 162.7, 162.3, 147.0, 132.3, 128.7, 127.7, 116.8, 116.4, 116.2, 114.5, 106.0, 55.5 |
| 75 | 2-(5-(4-(tert- butyl)phenyl)- 1,3,4-oxadiazol- 2-yl)aniline | 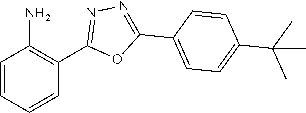 [SMILES: CC(C)(C)c1ccc(-c2nnc(-c3ccccc3N)o2)cc1] | 294.1 | ¹H NMR (400 MHz, CDCl₃) δ 8.07 (d, J = 7.0 Hz, 2H), 7.86 (d, J = 7.9 Hz, 1H), 7.55 (d, J = 7.0 Hz, 2H), 7.32- 7.24 (m, 1H), 6.87-6.74 (m, 2H), 5.91 (s, 2H), 1.38 (s, 9H). ¹³C{1H} NMR (100 MHz, CDCl₃) δ 164.4, 162.9, 155.3, 147.1, 132.4, 127.8, 126.8, 126.1, 121.1, 116.8, 116.2, 105.9, 35.1, 31.1 |
| 76 | 2-(5- methylfuran-2- yl)-5-(2- nitrophenyl)- 1,3,4-oxadiazole | 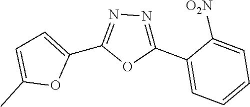 [SMILES: Cc1ccc(-c2nnc(-c3ccccc3[N+](=O)[O-])o2)o1] | 272.1 | ¹H NMR (500 MHz, CDCl₃) δ 8.05 (ddt, J = 19.9, 7.4, 1.6 Hz, 2H), 7.78 (dtt, J = 19.4, 7.7, 1.3 Hz, 2H), 7.10 (d, J = 3.4 Hz, 1H), 6.22 (dt, J = 3.4, 1.1 Hz, 1H), 2.44 (s, 3H). |
| 77 | 4-methoxy-2-(5- phenyl-1,3,4- oxadiazol-2- yl)aniline | 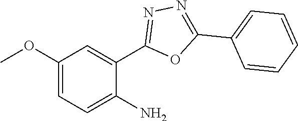 [SMILES: COc1ccc(N)c(-c2nnc(-c3ccccc3)o2)c1] | 268.1 | ¹H NMR (400 MHz, CDCl₃) δ 8.19-8.10 (m, 2H), 7.59- 7.50 (m, 3H), 7.36 (d, J = 2.7 Hz, 1H), 6.96 (dd, J = 8.9, 2.8 Hz, 1H), 6.79 (d, J = 8.9 Hz, 1H), 5.57 (br s, 2H), 3.84 (s, 3H). ¹³C{1H} NMR (100 MHz, CDCl₃) δ 164.5, 162.8, 151.2, 141.8, 131.7, 129.1, 127.0, 123.9, 120.7, 117.9, 111.1, 105.9, 56.1 |
| 78 | 2-methoxy-6-(5- phenyl-1,3,4- oxadiazol-2- yl)aniline | 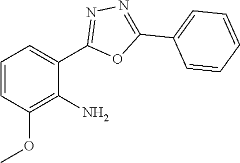 [SMILES: COc1cccc(-c2nnc(-c3ccccc3)o2)c1N] | 268.1 | ¹H NMR (400 MHz, CDCl₃) δ 8.23-8.07 (m, 2H), 7.61- 7.41 (m, 4H), 6.92-6.82 (m, 1H), 6.72 (t, J = 8.0 Hz, 1H), 6.04 (br s, 2H), 3.89 (s, 3H). ¹³C{1H} NMR (100 MHz, CDCl₃) δ 164.7, 162.6, 147.1, 138.2, 131.6, 129.0, 126.9, 123.9, 119.2, 115.7, 111.6, 105.1, 55.7 |
| 79 | 2-chloro-6-(5- phenyl-1,3,4- oxadiazol-2- yl)aniline | 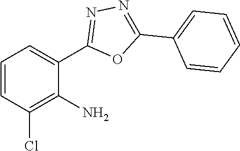 [SMILES: Nc1c(Cl)cccc1-c1nnc(-c2ccccc2)o1] | 272.1 | ¹H NMR (400 MHz, CDCl₃) δ 8.19-8.09 (m, 2H), 7.80 (dd, J = 8.0, 1.4 Hz, 1H), 7.64-7.49 (m, 3H), 7.41 (dd, J = 7.8, 1.5 Hz, 1H), 6.73 (t, J = 7.9 Hz, 1H), 6.45 (br s, 2H). ¹³C{1H} NMR (100 MHz, CDCl₃) δ 164.0, 163.1, 143.4, 132.2, 131.9, 129.1, 127.0, 126.4, 123.7, 120.1, 116.5, 106.8 |
| 80 | 2-(2- nitrophenyl)-5- phenyl-1,3,4- oxadiazole | 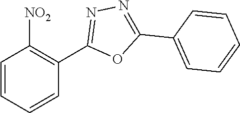 [SMILES: O=[N+]([O-])c1ccccc1-c1nnc(-c2ccccc2)o1] | 268.1 | ¹H NMR (400 MHz, CDCl₃): 8 8.15-8.01 (m, 4H), 7.84-7.72 (m, 2H), 7.62-7.47 (m, 3H). ¹³C{1H} NMR (100 MHz, CDCl₃): 8 174.35, 165.73, 161.23, 148.20, 133.07, 132.49, 132.15, 131.66, 129.13, 127.10, 124.66, 123.28, 118.60 |
| 81 | 2-(3- nitrophenyl)-5- phenyl-1,3,4- oxadiazole | 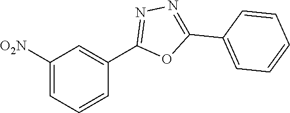 [SMILES: O=[N+]([O-])c1cccc(-c2nnc(-c3ccccc3)o2)c1] | 268.1 | ¹H NMR (400 MHz, CDCl₃) δ 8.95 (t, J = 2.0 Hz, 1H), 8.57-8.47 (m, 1H), 8.47- 8.36 (m, 1H), 8.17 (dd, J = 7.3, 2.0 Hz, 2H), 7.77 (t, J = 8.0 Hz, 1H), 7.58 (q, J = 6.9 Hz, 3H). ¹³C{1H} NMR (100 MHz, CDCl₃) δ 174.3, 165.3, 162.6, 148.6, 132.4, 132.2, 130.4, 129.2, 127.1, 126.1, 125.5, 123.3, 121.7. |
| 82 | 2-(4- methoxyphenyl)- 5-(2- nitrophenyl)- 1,3,4-oxadiazole | 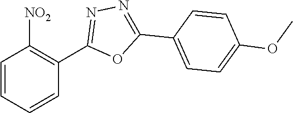 [SMILES: COc1ccc(-c2nnc(-c3ccccc3[N+](=O)[O-])o2)cc1] | 298 | ¹H NMR (CDCl₃) δ 8.25- 8.22 (m, 1H), 8.20-8.18 (m, 1H), 8.05-7.95 (m, 4H), 7.25-7.21 (m, 2H), 3.90 (s, 3H). |
| 83 | 2-(4-(tert- butyl)phenyl)-5- (2-nitrophenyl)- 1,3,4-oxadiazole | 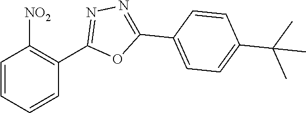 [SMILES: CC(C)(C)c1ccc(-c2nnc(-c3ccccc3[N+](=O)[O-])o2)cc1] | 324.1 | ¹H NMR (400 MHz, CDCl₃): 8 8.13-8.07 (m, 1H), 8.03 (dd, J = 7.9, 1.4 Hz, 1H), 8.02-7.96 (m, 2H), 7.83-7.71 (m, 2H), 7.58-7.49 (m, 2H), 1.36 (s, 9H). ¹³C{1H} NMR (100 MHz, CDCl₃): 8 174.36, 165.83, 160.97, 155.83, 148.23, 133.01, 132.39, 131.61, 126.95, 126.13, 124.60, 120.44, 118.68, 35.11, 31.07 |
| 84 | 2-(5-bromo-2- nitrophenyl)-5- phenyl-1,3,4- oxadiazole | 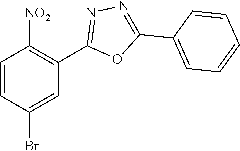 [SMILES: O=[N+]([O-])c1ccc(Br)cc1-c1nnc(-c2ccccc2)o1] | 347.1 | ¹H NMR (400 MHz, CDCl₃): δ 8.23 (d, J = 2.0 Hz, 1H), 8.06 (dd, J = 8.1, 1.6 Hz, 2H), 7.94 (d, J = 8.6 Hz, 1H), 7.88 (dd, J = 8.7, 2.0 Hz, 1H), 7.64-7.48 (m, 3H). ¹³C {1H} NMR (100 MHz, CDCl₃): 8 165.98, 160.09, 146.82, 135.44, 134.43, 132.34, 129.19, 127.80, 127.17, 126.10, 123.05, 120.22 |
| 85 | 2-(4-bromo-2- nitrophenyl)-5- phenyl-1,3,4- oxadiazole | 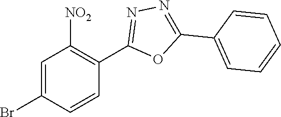 [SMILES: O=[N+]([O-])c1cc(Br)ccc1-c1nnc(-c2ccccc2)o1] | 347.1 | ¹H NMR (400 MHz, CDCl₃) δ 8.15 (d, J = 1.9 Hz, 1H), 8.09-8.03 (m, 2H), 8.00 (d, J = 8.3 Hz, 1H), 7.92 (dd, J = 8.3, 1.8 Hz, 1H), 7.62-7.48 (m, 3H). ¹³C{1H} NMR (100 MHz, CDCl₃) δ 174.3, 165.8, 160.5, 136.1, 132.4, 132.3, 129.2, 127.7, 127.1, 126.4, 123.1, 117.1 |
| 86 | 2-(4-methoxy-2- nitrophenyl)-5- phenyl-1,3,4- oxadiazole | 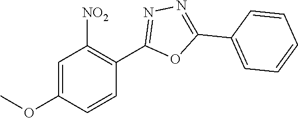 [SMILES: COc1ccc(-c2nnc(-c3ccccc3)o2)c([N+](=O)[O-])c1] | 298.1 | ¹H NMR (400 MHz, CDCl₃) δ 8.05 (dt, J = 6.8, 1.6 Hz, 2H), 8.00 (d, J = 8.6 Hz, 1H), 7.59-7.46 (m, 4H), 7.26 (dd, J = 8.7, 2.6 Hz, 1H), 3.96 (s, 3H). ¹³C{1H} NMR (100 MHz, CDCl₃) 8 165.4, 162.4, 161.2, 149.3, 132.7, 131.9, 129.1, 127.1, 123.4, 118.5, 110.4, 110.1, 56.2 |
| 87 | 2-(5-methoxy-2- nitrophenyl)-5- phenyl-1,3,4- oxadiazole | 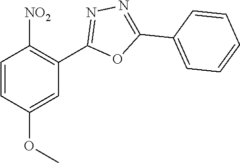 [SMILES: COc1ccc([N+](=O)[O-])c(-c2nnc(-c3ccccc3)o2)c1] | 298.1 | ¹H NMR (400 MHz, CDCl₃) δ 8.18 (d, J = 9.2 Hz, 1H), 8.12-8.02 (m, 2H), 7.61- 7.48 (m, 4H), 7.42 (d, J = 2.8 Hz, 1H), 7.18 (dd, J = 9.1, 2.8 Hz, 1H), 3.97 (s, 3H). ¹³C{1H} NMR (100 MHz, CDCl₃) δ 174.3, 163.1, 161.7, 132.1, 129.1, 129.0, 127.5, 127.1, 126.9, 123.3, 121.5, 117.2, 116.9, 56.4 |
| 88 | 2-(3-methoxy-2- nitrophenyl)-5- phenyl-1,3,4- oxadiazole | 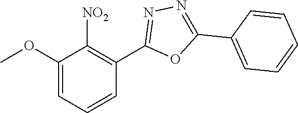 [SMILES: COc1cccc(-c2nnc(-c3ccccc3)o2)c1[N+](=O)[O-]] | 298.1 | ¹H NMR (400 MHz, CDCl₃) δ 8.06 (d, J = 7.5 Hz, 2H), 7.86 (d, J = 7.9 Hz, 1H), 7.65- 7.46 (m, 4H), 7.26 (d, J = 8.6 Hz, 1H), 3.98 (s, 3H). ¹³C{1H} NMR (100 MHz, CDCl₃) δ 174.3, 165.4, 160.1, 151.2, 132.2, 131.4, 129.1, 127.1, 123.1, 120.3, 116.8, 115.6, 56.7 |
| 89 | 2-(3-methyl-2- nitrophenyl)-5- phenyl-1,3,4- oxadiazole | 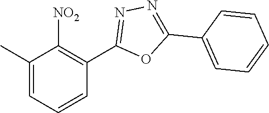 [SMILES: Cc1cccc(-c2nnc(-c3ccccc3)o2)c1[N+](=O)[O-]] | 282.1 | ¹H NMR (400 MHz, CDCl₃) δ 8.08 (ddd, J = 16.4, 8.3, 1.8 Hz, 3H), 7.62-7.47 (m, 5H), 2.43 (s, 3H). ¹³C {1H} NMR (100 MHz, CDCl₃) 8 165.3, 160.5, 148.8, 134.6, 132.1, 130.9, 130.4, 129.1, 127.2, 127.1, 123.2, 116.1, 17.2 |
| 90 | 2-(3-chloro-2- nitrophenyl)-5- phenyl-1,3,4- oxadiazole | 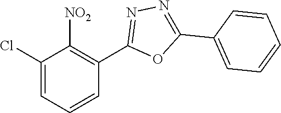 [SMILES: O=[N+]([O-])c1c(Cl)cccc1-c1nnc(-c2ccccc2)o1] | 302.1 | ¹H NMR (400 MHz, CDCl₃) δ 8.25 (dd, J = 8.0, 1.3 Hz, 1H), 8.11-8.02 (m, 2H), 7.73 (dd, J = 8.2, 1.3 Hz, 1H), 7.63 (t, J = 8.1 Hz, 1H), 7.55 (qd, J = 7.9, 3.8 Hz, 3H). ¹³C{1H} NMR (100 MHz, CDCl₃) δ 165.7, 159.4, 133.5, 132.4, 131.2, 129.2, 127.69, 127.2, 126.7, 122.9, 117.7 |
| 91 | 2-ethyl-5-(2- nitrophenyl)- 1,3,4-oxadiazole | 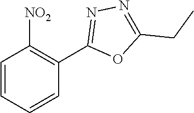 [SMILES: CCc1nnc(-c2ccccc2[N+](=O)[O-])o1] | 220.1 | ¹H NMR (400 MHz, CDCl₃) δ 8.02 (dd, J = 7.0, 1.2 Hz, 1H), 8.00-7.94 (m, 1H), 7.74 (tt, J = 9.4, 6.9 Hz, 2H), 2.94 (q, J = 7.6 Hz, 2H), 1.41 (t, J = 7.6 Hz, 3H). ¹³C {1H} NMR (100 MHz, CDCl₃) 8 169.0, 161.4, 133.1, 132.3, 131.6, 124.6, 118.9, 19.0, 10.7 |
| 92 | 2-(4- chlorophenyl)-5- (2-nitrophenyl)- 1,3,4-oxadiazole | 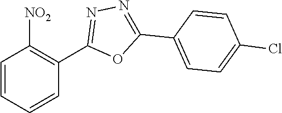 [SMILES: O=[N+]([O-])c1ccccc1-c1nnc(-c2ccc(Cl)cc2)o1] | 302.1 | ¹H NMR (400 MHz, CDCl₃) δ 8.07 (ddd, J = 7.9, 6.4, 1.7 Hz, 2H), 8.04-7.95 (m, 2H), 7.85-7.73 (m, 2H), 7.56-7.47 (m, 2H). ¹³C{1H} NMR (100 MHz, CDCl₃) δ 174.3, 164.9, 161.4, 148.1, 138.5, 133.1, 132.6, 131.7, 129.5, 129.5, 128.3, 128.2, 124.7, 121.7, 118.4 |
| 93 | 2-methyl-6-(5- phenyl-1,3,4- oxadiazol-2- yl)aniline | 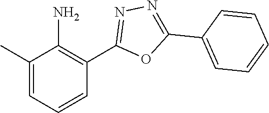 [SMILES: Cc1cccc(-c2nnc(-c3ccccc3)o2)c1N] | 252.1 | ¹H NMR (400 MHz, CDCl₃) δ 8.18-8.12 (m, 2H), 7.78 (d, J = 7.8 Hz, 1H), 7.58- 7.52 (m, 3H), 7.21 (d, J = 7.1 Hz, 1H), 6.75 (t, J = 7.6 Hz, 1H), 5.97 (br s, 2H), 2.26 (s, 3H). ¹³C{1H} NMR (100 MHz, CDCl₃) δ 165.0, 162.8, 145.6, 133.3, 131.6, 129.1, 126.9, 125.8, 124.0, 122.9, 116.5, 105.4, 17.6 |
| 94 | 2-(5-ethyl-1,3,4- oxadiazol-2- yl)aniline | 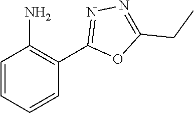 | 190.1 | ¹H NMR (400 MHz, CDCl₃) δ 7.87 (d, J = 7.4 Hz, 1H), 7.26 (t, J = 7.7 Hz, 1H), 6.69- 6.58 (m, 2H), 5.71 (br s, 2H), 4.33 (q, J = 7.1 Hz, 2H), 1.38 (t, J = 7.1 Hz, 3H). ¹³C{1H} NMR (100 MHz, CDCl₃) δ 168.1, 150.4, 134.0, 131.2, 131.2, 116.6, 116.2, 111.1, 60.3, 14.3 |
| 95 | 2-(5-(4- chlorophenyl)- 1,3,4-oxadiazol- 2-yl)aniline | 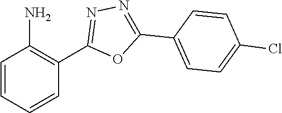 [SMILES: Nc1ccccc1-c1nnc(-c2ccc(Cl)cc2)o1] | 272.1 | ¹H NMR (400 MHz, CDCl₃) δ 8.08 (d, J = 8.7 Hz, 2H), 7.84 (dd, J = 7.9, 1.5 Hz, 1H), 7.52 (d, J = 8.8 Hz, 2H), 7.32-7.27 (m, 1H), 6.86-6.73 (m, 2H), 5.89 (br s, 2H). ¹³C{1H} NMR (100 MHz, CDCl₃) δ 164.8, 162.0, 147.2, 137.9, 132.7, 129.5, 128.2, 127.8, 122.4, 116.9, 116.3, 105.5 |
| 96 | 2-phenyl-5-(p- tolyl)-1,3,4- oxadiazole | 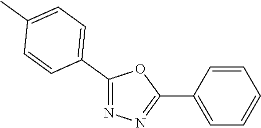 [SMILES: Cc1ccc(-c2nnc(-c3ccccc3)o2)cc1] | 237.1 | ¹H NMR (500 MHz, CDCl₃) δ 8.16 (dq, J = 6.1, 1.7 Hz, 2H), 8.07-8.01 (m, 2H), 7.55 (d, J = 6.1 Hz, 3H), 7.35 (d, J = 7.7 Hz, 2H), 2.46 (s, 3H). |
| 97 | 4-(5-(4- methoxyphenyl)- 1,3,4-oxadiazol- 2-yl)phenol | 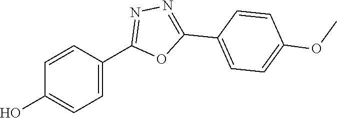 [SMILES: COc1ccc(-c2nnc(-c3ccc(O)cc3)o2)cc1] | 269.1 | ¹H NMR (500 MHz, acetone) δ 8.08 (dd, J = 8.9, 1.5 Hz, 2H), 8.04-7.98 (m, 2H), 7.18-7.12 (m, 2H), 7.08-7.02 (m, 2H), 3.95- 3.89 (m, 3H). |
| 98 | 4-(5-phenyl- 1,3,4-oxadiazol- 2-yl)phenol | 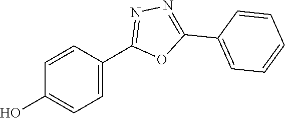 [SMILES: Oc1ccc(-c2nnc(-c3ccccc3)o2)cc1] | 239.1 | ¹H NMR (500 MHz, acetone) δ 8.19-8.12 (m, 1H), 8.04 (dd, J = 8.9, 1.2 Hz, 1H), 7.96-7.88 (m, 3H), 7.62 (ddd, J = 5.0, 2.1, 1.1 Hz, 1H), 7.10-7.01 (m, 1H), 6.95-6.88 (m, 3H). |
| 99 | 4-(5-(4- chlorophenyl)- 1,3,4-oxadiazol- 2-yl)phenol | 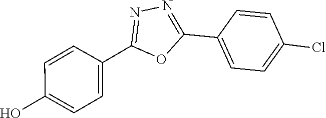 [SMILES: Oc1ccc(-c2nnc(-c3ccc(Cl)cc3)o2)cc1] | 273.0 | ¹H NMR (400 MHz, dmso) δ 10.85 (s, 1H), 8.14-8.08 (m, 1H), 8.06-8.00 (m, 2H), 7.96 (dd, J = 8.8, 2.6 Hz, 1H), 7.69 (dt, J = 8.7, 1.9 Hz, 1H), 6.98 (dd, J = 8.9, 2.5 Hz, 3H). |
| 100 | 4-(5-(p-tolyl)- 1,3,4-oxadiazol- 2-yl)phenol | 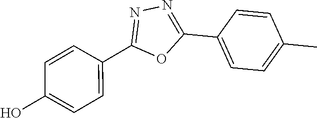 [SMILES: Cc1ccc(-c2nnc(-c3ccc(O)cc3)o2)cc1] | 253.1 | ¹H NMR (500 MHz, acetone) δ 8.08 (dd, J = 8.9, 1.5 Hz, 2H), 8.04-7.98 (m, 2H), 7.18-7.12 (m, 2H), 7.08-7.02 (m, 2H), 3.95- 3.89 (m, 3H). |
| 101 | 6-(5-(4- hydroxyphenyl)- 1,3,4-oxadiazol- 2-yl)naphthalen- 2-ol | 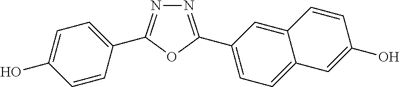 [SMILES: Oc1ccc(-c2nnc(-c3ccc4cc(O)ccc4c3)o2)cc1] | 305.1 | |
| 102 | 2-(naphthalen-2- yl)-5-(p-tolyl)- 1,3,4-oxadiazole | 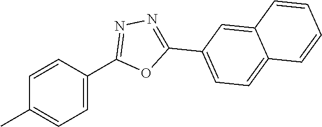 [SMILES: Cc1ccc(-c2nnc(-c3ccc4ccccc4c3)o2)cc1] | 287.1 | ¹H NMR (500 MHz, CDCl₃) δ 8.65 (s, 1H), 8.24 (dt, J = 8.6, 1.8 Hz, 1H), 8.10 (dd, J = 8.2, 1.8 Hz, 2H), 8.03- 7.98 (m, 2H), 7.93 (dd, J = 7.1, 2.3 Hz, 1H), 7.61 (dq, J = 6.2, 2.1 Hz, 2H), 7.41- 7.35 (m, 2H), 2.48 (d, J = 1.8 Hz, 3H). |
| 103 | 4-(5-(naphthalen- 2-yl)-1,3,4- oxadiazol-2- yl)phenol | 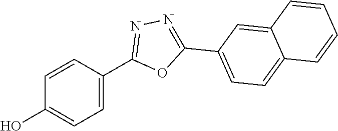 [SMILES: Oc1ccc(-c2nnc(-c3ccc4ccccc4c3)o2)cc1] | 289.1 | ¹H NMR (399 MHz, CDCl₃) δ 8.61 (s, 1H), 8.21 (d, J = 8.8 Hz, 1H), 8.10 (d, J = 8.0 Hz, 2H), 7.98 (d, J = 8.3 Hz, 2H), 7.90 (s, 1H), 7.62- 7.55 (m, 2H), 7.01 (d, J = 8.3 Hz, 2H). |
| 104 | 4-(5-(quinolin-2- yl)-1,3,4- oxadiazol-2- yl)phenol | 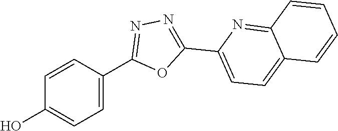 [SMILES: Oc1ccc(-c2nnc(-c3ccc4ccccc4n3)o2)cc1] | 290.1 | |
| 105 | methyl 2-(5-(2- chlorophenyl)- 1,3,4-oxadiazol- 2-yl)benzoate | 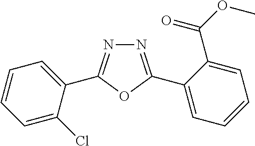 [SMILES: COC(=O)c1ccccc1-c1nnc(-c2ccccc2Cl)o1] | 315.1 | ¹H NMR (399 MHz, CDCl₃) δ 8.10-8.05 (m, 1H), 7.94 (td, J = 6.8, 2.1 Hz, 2H), 7.71- 7.62 (m, 2H), 7.57 (dt, J = 8.0, 1.2 Hz, 1H), 7.51-7.45 (m, 1H), 7.42 (tt, J = 7.5, 1.3 Hz, 1H), 3.86 (d, J = 1.1 Hz, 3H). |
| 106 | methyl 3-(5-(2- chlorophenyl)- 1,3,4-oxadiazol- 2-yl)benzoate | 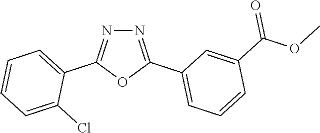 [SMILES: COC(=O)c1cccc(-c2nnc(-c3ccccc3Cl)o2)c1] | 315.1 | ¹H NMR (399 MHz, CDCl₃) δ 8.70-8.67 (m, 1H), 8.26 (dq, J = 7.8, 1.1 Hz, 1H), 8.14 (ddd, J = 7.7, 1.9, 1.0 Hz, 1H), 8.05-8.02 (m, 1H), 7.58-7.48 (m, 2H), 7.45-7.34 (m, 2H), 3.91 (d, J = 1.1 Hz, 3H). ¹³C NMR (100 MHz, CDCl₃) δ 165.85, 164.16, 163.18, 133.06, 132.64, 132.53, 131.28, 131.21, 131.17, 131.02, 129.34, 127.93, 127.12, 124.10, 122.86, 52.47, 52.44, 29.65. |
| 107 | methyl 4-(5-(2- chlorophenyl)- 1,3,4-oxadiazol- 2-yl)benzoate | 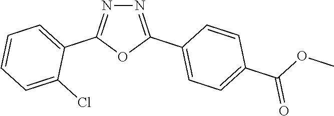 [SMILES: COC(=O)c1ccc(-c2nnc(-c3ccccc3Cl)o2)cc1] | 315.1 | ¹H NMR (500 MHz, CDCl₃) δ 8.24-8.19 (m, 5H), 8.14- 8.10 (m, 1H), 7.59 (dq, J = 8.0, 1.2 Hz, 1H), 7.51 (tdd, J = 8.1, 2.6, 1.7 Hz, 1H), 7.45 (tq, J = 7.5, 1.3 Hz, 1H), 3.98- 3.96 (m, 3H). |
| 108 | 2-(4- butoxyphenyl)-5- (2-chlorophenyl)- 1,3,4-oxadiazole | 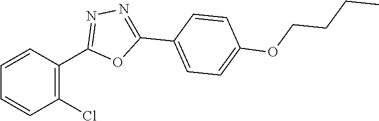 [SMILES: CCCCOc1ccc(-c2nnc(-c3ccccc3Cl)o2)cc1] | 329.0 | ¹H NMR (399 MHz, dmso) δ 8.12 (dd, J = 7.7, 2.0 Hz, 1H), 8.05-7.99 (m, 2H), 7.76-7.72 (m, 1H), 7.66 (td, J = 7.7, 2.0 Hz, 1H), 7.59 (dd, J = 8.3, 6.5 Hz, 1H), 7.20-7.14 (m, 2H), 4.08 (t, J = 6.5 Hz, 2H), 1.73 (p, J = 6.9 Hz, 2H), 1.45 (h, J = 7.5 Hz, 2H), 0.99-0.91 (m, 3H). ¹³C NMR (100 MHz, dmso) δ 164.78, 162.19, 132.19, 131.75, 131.63, 129.08, 123.04, 115.83, 115.70, 31.03, 19.13, 14.13. |
| 109 | 2-(1-phenyl-1H- pyrazol-4-yl)-5- (p-tolyl)-1,3,4- oxadiazole | 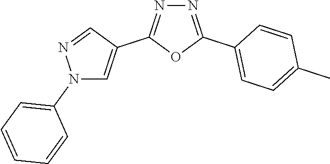 [SMILES: Cc1ccc(-c2nnc(-c3cnn(-c4ccccc4)c3)o2)cc1] | 303.1 | ¹H NMR (399 MHz, CDCl₃) δ 8.60 (d, J = 0.9 Hz, 1H), 8.29 (s, 1H), 8.02 (d, J = 7.9 Hz, 2H), 7.77 (dd, J = 7.8, 2.1 Hz, 2H), 7.52 (t, J = 7.7 Hz, 2H), 7.42-7.38 (m, 1H), 7.34 (d, J = 7.9 Hz, 2H), 2.45 (s, 3H). ¹³C NMR (100 MHz, CDCl₃) δ 196.10, 151.50, 142.28, 139.78, 129.79, 129.70, 127.69, 126.84, 119.57, 77.32, 77.00, 76.68, 57.41, 29.71. |
| 110 | (4-(5-(2- chlorophenyl)- 1,3,4-oxadiazol- 2- yl)phenyl) boronic acid | 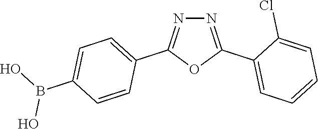 [SMILES: OB(O)c1ccc(-c2nnc(-c3ccccc3Cl)o2)cc1] | 301.1 | ¹H NMR (500 MHz, CDCl₃) δ 8.11 (dt, J = 7.7, 1.5 Hz, 1H), 7.89 (tdd, J = 8.8, 7.7, 1.2 Hz, 4H), 7.59 (dt, J = 8.0, 1.3 Hz, 1H), 7.51 (tt, J = 7.9, 1.5 Hz, 1H), 7.45 (ddt, J = 8.8, 7.5, 1.3 Hz, 1H). |
| 111 | (4-(5-(p-tolyl)- 1,3,4-oxadiazol- 2- yl)phenyl) boronic acid | 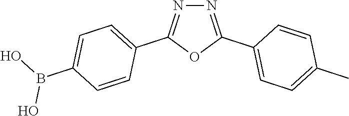 [SMILES: Cc1ccc(-c2nnc(-c3ccc(B(O)O)cc3)o2)cc1] | 281.1 | ¹H NMR (500 MHz, CDCl₃) δ 8.01 (td, J = 6.4, 2.4 Hz, 2H), 7.87 (dtd, J = 15.0, 8.6, 7.6, 4.1 Hz, 4H), 7.33 (t, J = 6.8 Hz, 2H), 2.44 (d, J = 5.7 Hz, 3H). |
| 112 | (4-(5-(4- methoxyphenyl)- 1,3,4-oxadiazol- 2- yl)phenyl) boronic acid | 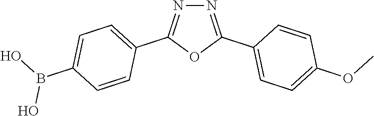 [SMILES: COc1ccc(-c2nnc(-c3ccc(B(O)O)cc3)o2)cc1] | 297.1 | ¹H NMR (400 MHz, CDCl₃) δ 8.09-8.02 (m, 2H), 7.91- 7.78 (m, 4H), 7.06-6.97 (m, 2H), 3.98-3.77 (m, 4H). |
| 113 | 2-(2- chlorophenyl)-5- (1-phenyl-1H- pyrazol-4-yl)- 1,3,4-oxadiazole | 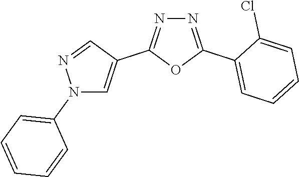 [SMILES: Clc1ccccc1-c1nnc(-c2cnn(-c3ccccc3)c2)o1] | 323.1 | |
| 114 | 2-(quinolin-5-yl)- 5-(p-tolyl)-1,3,4- oxadiazole | 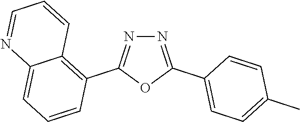 [SMILES: Cc1ccc(-c2nnc(-c3cccc4ncccc34)o2)cc1] | 288.1 | ¹H NMR (399 MHz, CDCl₃) δ 9.25-9.18 (m, 1H), 8.74 (dq, J = 6.2, 1.1 Hz, 1H), 8.59 (dd, J = 6.2, 2.5 Hz, 1H), 8.02-7.92 (m, 2H), 7.84-7.79 (m, 2H), 7.55 (td, J = 8.0, 3.9 Hz, 1H), 7.20 (dd, J = 8.3, 3.2 Hz, 2H), 2.33 (d, J = 2.8 Hz, 3H). ¹³C NMR (100 MHz, CDCl₃) 8 168.78, 165.70, 152.86, 144.93, 141.86, 133.50, 132.93, 132.92, 130.83, 129.88, 128.85, 127.86, 126.96, 126.50, 125.94, 125.92, 118.62, 77.38, 21.52, 21.50. |
| 115 | 2-(2- chlorophenyl)-5- (quinolin-5-yl)- 1,3,4-oxadiazole | 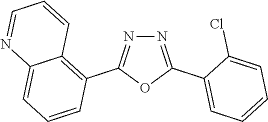 [SMILES: Clc1ccccc1-c1nnc(-c2cccc3ncccc23)o1] | 308.1 | ¹H NMR (399 MHz, CDCl₃) δ 9.33 (d, J = 2.6 Hz, 1H), 8.76 (dd, J = 6.2, 2.5 Hz, 1H), 8.66 (dd, J = 6.2, 2.6 Hz, 1H), 8.44 (dt, J = 6.5, 3.2 Hz, 1H), 8.12 (dt, J = 7.0, 3.0 Hz, 2H), 7.69 (td, J = 7.7, 2.5 Hz, 1H), 7.55 (dt, J = 6.5, 3.1 Hz, 1H), 7.45 (dq, J = 6.5, 3.5 Hz, 2H). ¹³C NMR (100 MHz, CDCl₃) δ 167.57, 164.04, 152.96, 145.07, 133.60, 133.18, 132.69, 131.86, 131.08, 131.05, 130.67, 128.97, 128.75, 127.49, 126.58, 125.91, 118.55, 77.25, 29.68, 1.01. |
| 116 | 2-(thiazol-2-yl)- 5-(p-tolyl)-1,3,4- oxadiazole | 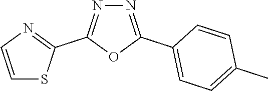 [SMILES: Cc1ccc(-c2nnc(-c3nccs3)o2)cc1] | 244.0 | ¹H NMR (399 MHz, CDCl₃) δ 9.00 (d, J = 3.0 Hz, 1H), 8.58 (d, J = 3.0 Hz, 1H), 8.05- 7.97 (m, 2H), 7.38-7.31 (m, 2H), 2.45 (d, J = 2.8 Hz, 3H). ¹³C NMR (100 MHz, CDCl₃) δ 164.88, 158.58, 156.13, 144.91, 142.87, 129.89, 127.05, 120.40, 77.32, 77.20, 77.00, 76.69, 21.69. |
| 117 | 2-(2- chlorophenyl)-5- (thiazol-2-yl)- 1,3,4-oxadiazole | 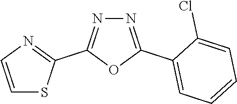 [SMILES: Clc1ccccc1-c1nnc(-c2nccs2)o1] | 264.1 | ¹H NMR (399 MHz, CDCl₃) δ 9.02-8.98 (m, 1H), 8.60- 8.56 (m, 1H), 8.09-7.98 (m, 1H), 7.61-7.56 (m, 1H), 7.51 (td, J = 7.7, 1.8 Hz, 1H), 7.44 (tt, J = 7.5, 1.2 Hz, 1H). |
| 118 | methyl 3-(5-(4- (trifluoromethyl) phenyl)-1,3,4- oxadiazol-2- yl)benzoate | 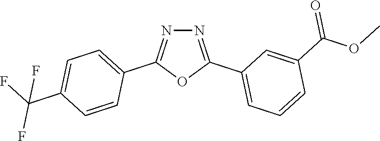 [SMILES: COC(=O)c1cccc(-c2nnc(-c3ccc(C(F)(F)F)cc3)o2)c1] | 349.1 | |
| 119 | methyl 2-(5-(4- (trifluoromethyl) phenyl)-1,3,4- oxadiazol-2- yl)benzoate | 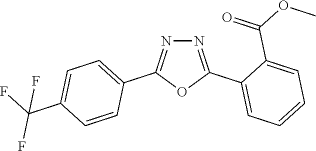 [SMILES: COC(=O)c1ccccc1-c1nnc(-c2ccc(C(F)(F)F)cc2)o1] | 349.1 | ¹H NMR (399 MHz, CDCl₃) δ 8.10-8.05 (m, 1H), 7.94 (td, J = 6.8, 2.1 Hz, 2H), 7.71- 7.62 (m, 2H), 7.57 (dt, J = 8.0, 1.2 Hz, 1H), 7.51-7.45 (m, 1H), 7.42 (tt, J = 7.5, 1.3 Hz, 1H), 3.86 (d, J = 1.1 Hz, 3H). |
| 120 | methyl 4-(5-(4- (trifluoromethyl) phenyl)-1,3,4- oxadiazol-2- yl)benzoate | 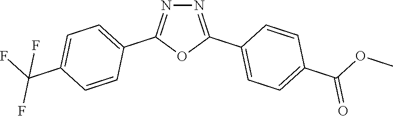 [SMILES: COC(=O)c1ccc(-c2nnc(-c3ccc(C(F)(F)F)cc3)o2)cc1] | 349.1 | ¹H NMR (399 MHz, CDCl₃) δ 8.28 (d, J = 8.1 Hz, 2H), 8.21 (s, 4H), 7.81 (d, J = 8.2 Hz, 2H), 3.97 (d, J = 2.7 Hz, 3H). ¹³C NMR (100 MHz, CDCl₃) δ 166.00, 164.36, 163.85, 133.74, 133.41, 133.15, 130.33, 127.35, 127.28, 126.96, 126.87, 126.26, 126.22, 126.18, 124.87, 122.16, 52.53, 52.50, 1.00. |
| 121 | (4-(5-(4- (trifluoromethyl) phenyl)-1,3,4- oxadiazol-2- yl)phenyl) boronic acid | 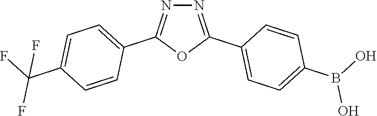 [SMILES: OB(O)c1ccc(-c2nnc(-c3ccc(C(F)(F)F)cc3)o2)cc1] | 335.1 | ¹H NMR (500 MHz, CDCl₃) δ 8.26 (d, J = 8.1 Hz, 2H), 7.92 (dd, J = 8.3, 1.6 Hz, 2H), 7.87 (dd, J = 8.5, 1.6 Hz, 2H), 7.81 (d, J = 8.1 Hz, 2H). |
| 122 | (4-(5-(4-(tert- butyl)phenyl)- 1,3,4-oxadiazol- 2- yl)phenyl) boronic acid | 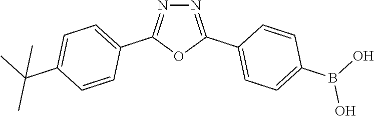 [SMILES: CC(C)(C)c1ccc(-c2nnc(-c3ccc(B(O)O)cc3)o2)cc1] | 323.1 | |
| 123 | 2-(6- methoxypyridin- 2-yl)-5-(3- (trifluoromethyl) phenyl)-1,3,4- oxadiazole | 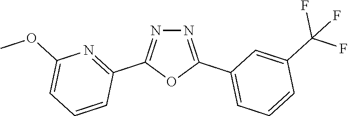 [SMILES: COc1cccc(-c2nnc(-c3cccc(C(F)(F)F)c3)o2)n1] | 322.1 | ¹H NMR (500 MHz, CDCl₃) δ 8.46-8.34 (m, 2H), 7.91- 7.80 (m, 2H), 7.80-7.67 (m, 2H), 6.95 (d, J = 8.2 Hz, 1H), 4.10 (d, J = 1.6 Hz, 3H). |
| 124 | 2-(6- methoxypyridin- 3-yl)-5-(3- (trifluoromethyl) phenyl)-1,3,4- oxadiazole | 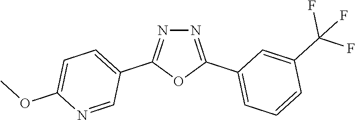 [SMILES: COc1ccc(-c2nnc(-c3cccc(C(F)(F)F)c3)o2)cn1] | 322.1 | |
| 125 | 2-(6- methoxypyridin- 3-yl)-5-(4- (trifluoromethyl) phenyl)-1,3,4- oxadiazole | 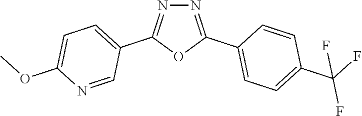 [SMILES: COc1ccc(-c2nnc(-c3ccc(C(F)(F)F)cc3)o2)cn1] | 322.1 | ¹H NMR (399 MHz, CDCl₃) δ 8.76 (s, 1H), 8.27 (dd, J = 9.0, 2.4 Hz, 1H), 8.14 (d, J = 8.1 Hz, 2H), 7.77 (d, J = 8.1 Hz, 2H), 6.90 (d, J = 8.7 Hz, 1H), 4.03 (s, 3H). |
| 126 | 2-(6- methylpyridin-2- yl)-5-(4- (trifluoromethyl) phenyl)-1,3,4- oxadiazole | 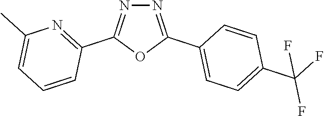 [SMILES: Cc1cccc(-c2nnc(-c3ccc(C(F)(F)F)cc3)o2)n1] | 306.1 | ¹H NMR (399 MHz, cdc13) 8 8.32 (s, 1H), 8.22 (dd, J = 13.7, 7.8 Hz, 2H), 7.76 (t, J = 7.9 Hz, 2H), 7.65 (t, J = 7.8 Hz, 1H), 7.28 (s, 1H), 2.63 (s, 3H). |
| 127 | 2-(6- methylpyridin-2- yl)-5-(3- (trifluoromethyl) phenyl)-1,3,4- oxadiazole | 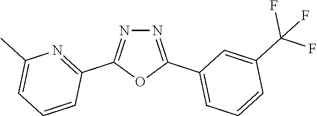 [SMILES: Cc1cccc(-c2nnc(-c3cccc(C(F)(F)F)c3)o2)n1] | 306.1 | ¹H NMR (399 MHz, Chloroform-d) δ 8.45 (s, 1H), 8.40 (d, J = 7.9 Hz, 1H), 8.11 (d, J = 7.7 Hz, 1H), 7.84-7.76 (m, 2H), 7.67 (t, J = 8.0 Hz, 1H), 7.35 (d, J = 7.8 Hz, 1H), 2.70 (d, J = 2.5 Hz, 3H). |
| 128 | 2-(2- chloropyridin-3- yl)-5-(3- (trifluoromethyl) phenyl)-1,3,4- oxadiazole | 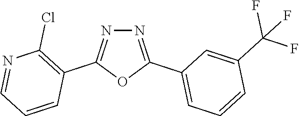 [SMILES: FC(F)(F)c1cccc(-c2nnc(-c3cccnc3Cl)o2)c1] | 326.0 | |
| 129 | 2-(2- chloropyridin-3- yl)-5-(4- (trifluoromethyl) phenyl)-1,3,4- oxadiazole | 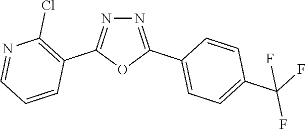 [SMILES: FC(F)(F)c1ccc(-c2nnc(-c3cccnc3Cl)o2)cc1] | 326.0 | |
| 130 | 3-(5-(2- chlorophenyl)- 1,3,4-oxadiazol- 2-yl)benzoic acid | 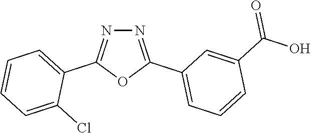 [SMILES: O=C(O)c1cccc(-c2nnc(-c3ccccc3Cl)o2)c1] | 301.1 | |
| 131 | 4-(5-(2- chlorophenyl)- 1,3,4-oxadiazol- 2-yl)benzoic acid | 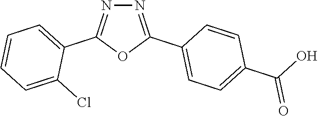 [SMILES: O=C(O)c1ccc(-c2nnc(-c3ccccc3Cl)o2)cc1] | 301.1 | |
| 132 | 4-(5-(naphthalen- 2-ylmethyl)- 1,3,4-oxadiazol- 2-yl)phenol | 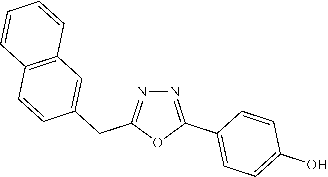 [SMILES: Oc1ccc(-c2nnc(Cc3ccc4ccccc4c3)o2)cc1] | 303.1 | |
| 133 | 2-(4- methylbenzyl)-5- (4-methylthiazol- 2-yl)-1,3,4- oxadiazole | 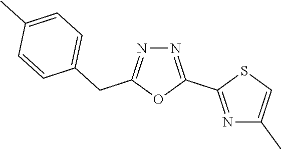 | 272.0 | |
| 134 | 2-(2- chlorophenyl)-5- (pyridin-4-yl)- 1,3,4-oxadiazole | 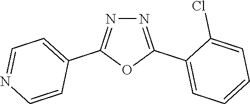 [SMILES: Clc1ccccc1-c1nnc(-c2ccncc2)o1] | 358.1 | ¹H NMR (400 MHz, CDCl₃) δ 8.88 (q, J = 5.8 Hz, 2H), 8.14 (dt, J = 11.8, 6.1 Hz, 1H), 8.03 (dt, J = 10.0, 5.0 Hz, 2H), 7.61 (dt, J = 12.8, 6.4 Hz, 1H), 7.55 (p, J = 6.0, 4.9 Hz, 1H), 7.50-7.43 (m, 1H). |
| 135 | 2-(4- methoxyphenyl)- 5-(pyridin-4-yl)- 1,3,4-oxadiazole | 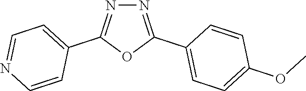 [SMILES: COc1ccc(-c2nnc(-c3ccncc3)o2)cc1] | 254.1 | ¹H NMR (500 MHz, CDCl₃) δ 8.86-8.79 (m, 2H), 8.11- 8.06 (m, 2H), 8.00-7.94 (m, 2H), 7.08-7.01 (m, 2H), 3.90 (s, 3H). |
| 136 | 2-(pyridin-4-yl)- 5-(p-tolyl)-1,3,4- oxadiazole | 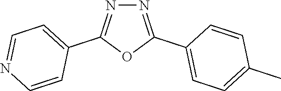 [SMILES: Cc1ccc(-c2nnc(-c3ccncc3)o2)cc1] | 238.1 | ¹H NMR (500 MHz, CDCl₃) δ 8.82 (d, J = 5.2 Hz, 2H), 8.02 (d, J = 7.9 Hz, 2H), 7.98- 7.92 (m, 2H), 7.34 (d, J = 7.9 Hz, 2H), 2.44 (s, 3H). |
| 137 | methyl 3-(5- (pyridin-4-yl)- 1,3,4-oxadiazol- 2-yl)benzoate | 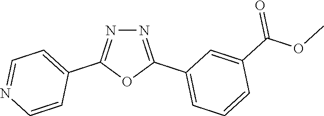 [SMILES: COC(=O)c1cccc(-c2nnc(-c3ccncc3)o2)c1] | 282.1 | ¹H NMR (400 MHz, CDCl₃) δ 8.86 (q, J = 4.5, 3.6 Hz, 2H), 8.76 (dd, J = 6.6, 2.8 Hz, 1H), 8.36 (t, J = 7.5 Hz, 1H), 8.29-8.18 (m, 1H), 8.02 (q, J = 4.5, 3.6 Hz, 2H), 7.65 (q, J = 8.0 Hz, 1H), 3.99 (d, J = 7.2 Hz, 3H). |
| 138 | 5-methoxy-2-(5- (pyridin-4-yl)- 1,3,4-oxadiazol- 2-yl)phenol | 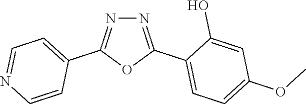 [SMILES: COc1ccc(-c2nnc(-c3ccncc3)o2)c(O)c1] | 270.0 | |
| 139 | 5-methyl-2-(5- (pyridin-4-yl)- 1,3,4-oxadiazol- 2-yl)phenol | 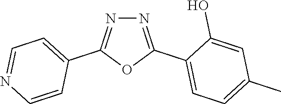 [SMILES: Cc1ccc(-c2nnc(-c3ccncc3)o2)c(O)c1] | 254.1 | |
| 140 | 4-(5-(naphthalen- 2-ylmethyl)- 1,3,4-oxadiazol- 2-yl)phenol | 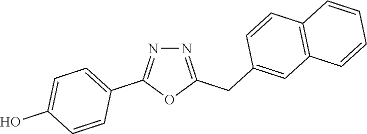 [SMILES: Oc1ccc(-c2nnc(Cc3ccc4ccccc4c3)o2)cc1] | 303.1 | |
| 141 | N-(4-(5-phenyl- 1,3,4-oxadiazol- 2- yl)phenyl) cyclopropane- carboxamide | 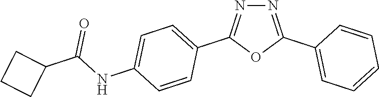 [SMILES: O=C(Nc1ccc(-c2nnc(-c3ccccc3)o2)cc1)C1CCC1] | 306.1 | |
| 142 | N-(4-(5-phenyl- 1,3,4-oxadiazol- 2- yl)phenyl) cyclohexane- carboxamide | 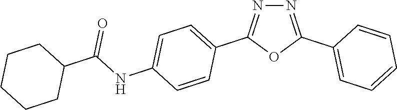 [SMILES: O=C(Nc1ccc(-c2nnc(-c3ccccc3)o2)cc1)C1CCCCC1] | 348.1 | |
| 143 | N-(4-(5-phenyl- 1,3,4-oxadiazol- 2- yl)phenyl) cyclobutane- carboxamide | 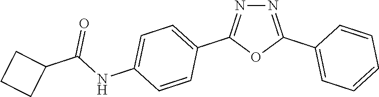 [SMILES: O=C(Nc1ccc(-c2nnc(-c3ccccc3)o2)cc1)C1CCC1] | 320.1 | |
| 144 | S-(5-phenyl- 1,3,4-oxadiazol- 2-yl) benzothioate | 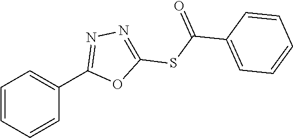 [SMILES: O=C(Sc1nnc(-c2ccccc2)o1)c1ccccc1] | 263.1 | |
| 145 | N-(5-phenyl- 1,3,4-oxadiazol- 2-yl)benzamide | 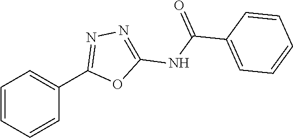 [SMILES: O=C(Nc1nnc(-c2ccccc2)o1)c1ccccc1] | 266.1 | ¹H NMR (500 MHz, CDCl₃) δ 11.98 (s, 1H), 8.29-8.23 (m, 2H), 8.13-8.07 (m, 2H), 7.64 (t, J = 7.4 Hz, 1H), 7.60-7.45 (m, 5H). |
| 146 | 4-methyl-N-(5- phenyl-1,3,4- oxadiazol-2- yl)benzamide | 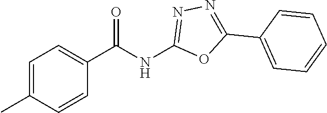 [SMILES: Cc1ccc(C(=O)Nc2nnc(-c3ccccc3)o2)cc1] | 280.1 | ¹H NMR (399 MHz, dmso) δ 7.97-7.91 (m, 4H), 7.48- 7.41 (m, 4H), 7.22 (d, J = 8.0 Hz, 2H), 2.35 (s, 3H). |
| 147 | methyl 2-(5-(3- (trifluoromethyl) phenyl)-1,3,4- oxadiazol-2- yl)benzoate | 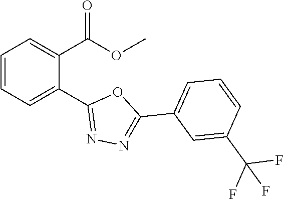 [SMILES: COC(=O)c1ccccc1-c1nnc(-c2cccc(C(F)(F)F)c2)o1] | | |
| 148 | methyl 3-(5-(3- (trifluoromethyl) phenyl)-1,3,4- oxadiazol-2- yl)benzoate | 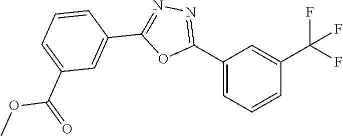 [SMILES: COC(=O)c1cccc(-c2nnc(-c3cccc(C(F)(F)F)c3)o2)c1] | | |
| 149 | methyl 4-(5-(3- (trifluoromethyl) phenyl)-1,3,4- oxadiazol-2- yl)benzoate | 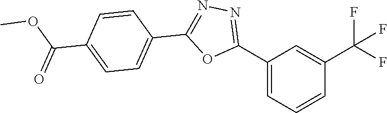 [SMILES: COC(=O)c1ccc(-c2nnc(-c3cccc(C(F)(F)F)c3)o2)cc1] | | |
| 150 | (2-(5-(3- (trifluoromethyl) phenyl)-1,3,4- oxadiazol-2- yl)phenyl) boronic acid | 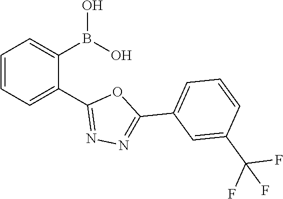 [SMILES: OB(O)c1ccccc1-c1nnc(-c2cccc(C(F)(F)F)c2)o1] | | |
| 151 | (3-(5-(3- (trifluoromethyl) phenyl)-1,3,4- oxadiazol-2- yl)phenyl) boronic acid | 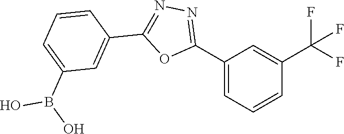 [SMILES: OB(O)c1cccc(-c2nnc(-c3cccc(C(F)(F)F)c3)o2)c1] | | |
| 152 | (4-(5-(3- (trifluoromethyl) phenyl)-1,3,4- oxadiazol-2- yl)phenyl) boronic acid | 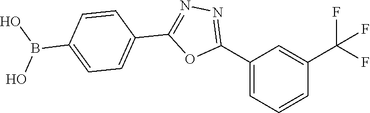 [SMILES: OB(O)c1ccc(-c2nnc(-c3cccc(C(F)(F)F)c3)o2)cc1] | | |
| 153 | (4-(5-(6- (trifluoromethyl) pyridin-3-yl)- 1,3,4-oxadiazol- 2- yl)phenyl) boronic acid | 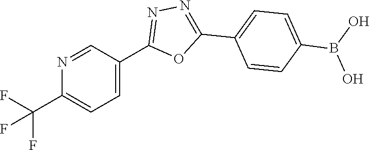 [SMILES: OB(O)c1ccc(-c2nnc(-c3ccc(C(F)(F)F)nc3)o2)cc1] | | |
| 154 | 2,5-Diphenyl- 1,3,4-oxadiazole | 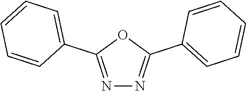 [SMILES: c1ccc(-c2nnc(-c3ccccc3)o2)cc1] | 223.1 | ¹H NMR (500 MHz, CDCl₃) δ 8.23-8.12 (m, 4H), 7.57 (dt, J = 18.7, 6.8 Hz, 6H). |
| 155 | 2-(4- Chlorophenyl)-5- phenyl-1,3,4- oxadiazole | 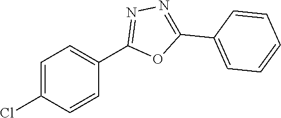 [SMILES: Clc1ccc(-c2nnc(-c3ccccc3)o2)cc1] | 257.1 | ¹H NMR (400 MHz, CDCl₃) δ 8.17-8.07 (m, 4H), 7.55 (dd, J = 13.2, 7.8 Hz, 5H) |
| 156 | 2-Phenyl-5- (thiophen-2-yl)- 1,3,4-oxadiazole | 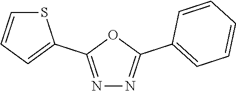 [SMILES: c1ccc(-c2nnc(-c3cccs3)o2)cc1] | 229.1 | ¹H NMR (400 MHz, CDCl₃) δ 8.15-8.10 (m, 2H), 7.85 (dt, J = 3.8, 1.4 Hz, 1H), 7.59 (dt, J = 5.0, 1.4 Hz, 1H), 7.57- 7.55 (m, 2H), 7.55-7.52 (m, 1H), 7.21 (ddd, J = 5.0, 3.7, 1.5 Hz, 1H). |
| 157 | 2,5-bis(4- chlorophenyl)- 1,3,4-oxadiazole | 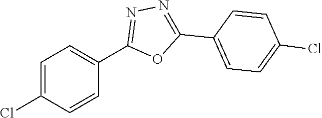 [SMILES: Clc1ccc(-c2nnc(-c3ccc(Cl)cc3)o2)cc1] | 292.1 | ¹H NMR (400 MHz, CDCl₃) δ 8.12-8.06 (m, 4H), 7.56- 7.51 (m, 4H). |
| 158 | 2-Phenyl-5-(o- tolyl)-1,3,4- oxadiazole | 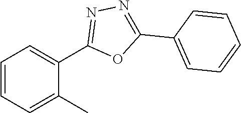 [SMILES: Cc1ccccc1-c1nnc(-c2ccccc2)o1] | 237.1 | ¹H NMR (500 MHz, CDCl₃) δ 8.04-7.99 (m, 2H), 7.93- 7.89 (m, 2H), 7.50 (tt, J = 3.5, 2.4 Hz, 3H), 7.33-7.28 (m, 2H), 2.43 (s, 3H). |
| 159 | 2-(4- Bromophenyl)-5- phenyl-1,3,4- oxadiazole | 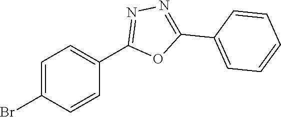 [SMILES: Brc1ccc(-c2nnc(-c3ccccc3)o2)cc1] | 302.1 | ¹H NMR (500 MHz, CDCl₃) δ 8.15 (ddd, J = 8.1, 3.5, 1.7 Hz, 2H), 8.05-7.99 (m, 2H), 7.72-7.66 (m, 2H), 7.56 (dtt, J = 9.6, 6.5, 2.5 Hz, 3H). |
| 160 | (4-(5-(4- hydroxyphenyl)- 1,3,4-oxadiazol- 2- yl)phenyl) boronic acid | 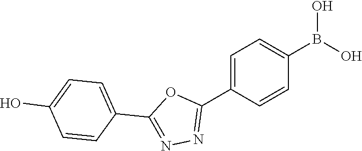 [SMILES: OB(O)c1ccc(-c2nnc(-c3ccc(O)cc3)o2)cc1] | 283.1 | |
| 1001 | 2-(4- methoxyphenyl)- 5-phenyl-1,3,4- thiadiazole | 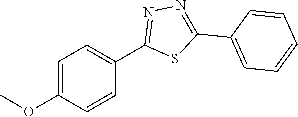 [SMILES: COc1ccc(-c2nnc(-c3ccccc3)s2)cc1] | 269.1 | ¹H NMR (500 MHz, CDCl₃) δ 8.08-8.00 (m, 2H), 7.08 (d, J = 3.4 Hz, 1H), 7.04- 6.98 (m, 2H), 6.21 (d, J = 3.4 Hz, 1H), 3.88 (d, J = 2.2 Hz, 3H), 2.44 (s, 3H). |
| 1002 | 2-phenyl-5-(p- tolyl)-1,3,4- thiadiazole | 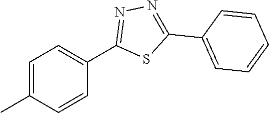 [SMILES: Cc1ccc(-c2nnc(-c3ccccc3)s2)cc1] | 253.1 | ¹H NMR (500 MHz, CDCl₃) δ 8.04-7.99 (m, 2H), 7.93- 7.88 (m, 2H), 7.50 (tt, J = 3.5, 2.4 Hz, 3H), 7.33-7.28 (m, 2H), 2.43 (s, 3H). ¹³C NMR (101 MHz, CDCl₃) 8 168.3, 167.8, 141.6, 131.1, 130.4, 129.9, 129.2, 128.0, 127.9, 127.5, 21.6. |
| 1003 | 2-(2- chlorophenyl)-5- (p-tolyl)-1,3,4- thiadiazole | 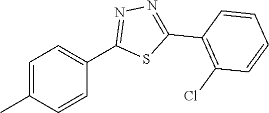 [SMILES: Cc1ccc(-c2nnc(-c3ccccc3Cl)s2)cc1] | 287.1 | ¹H NMR (500 MHz, CDCl₃) δ 8.40-8.34 (m, 1H), 7.97- 7.93 (m, 2H), 7.58-7.53 (m, 1H), 7.47-7.41 (m, 2H), 7.34-7.28 (m, 2H), 2.44 (s, 3H). |
| 1004 | 2,5-diphenyl- 1,3,4-thiadiazole | 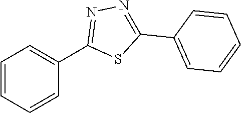 [SMILES: c1ccc(-c2nnc(-c3ccccc3)s2)cc1] | 239.1 | ¹H NMR (500 MHz, CDCl₃) δ 8.23-8.12 (m, 4H), 7.57 (dt, J = 18.7, 6.8 Hz, 6H). |
| 1005 | 2-(2- nitrophenyl)-5- (p-tolyl)-1,3,4- thiadiazole | 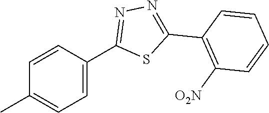 [SMILES: Cc1ccc(-c2nnc(-c3ccccc3[N+](=O)[O-])s2)cc1] | 297 | ¹H NMR (400 MHz, CDCl₃) δ 8.09 (dt, J = 7.5, 1.3 Hz, 1H), 8.06-7.95 (m, 3H), 7.84-7.70 (m, 2H), 7.06- 6.97 (m, 2H), 1.89 (s, 3H). |
| 1006 | 2-cyclohexyl-5- phenyl-1,3,4- thiadiazole | 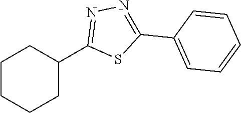 [SMILES: c1ccc(-c2nnc(C3CCCCC3)s2)cc1] | 245.1 | ¹H NMR (500 MHz, CDCl₃) δ 8.0-7.9 (m, 2H), 7.5-7.4 (m, 3H), 3.3-3.1 (m, 1H), 2.24-2.20 (m, 2H), 1.93-1.80 (m, 2H), 1.82-1.76 (m, 1H), 1.66-1.57 (m, 2H), 1.52-1.29 (m, 3H). |
| 1007 | 2-((1S,3S)- adamantan-1-yl)- 5-phenyl-1,3,4- thiadiazole | 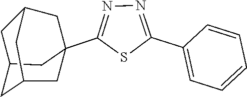 [SMILES: c1ccc(-c2nnc(C34CC5CC(CC(C5)C3)C4)s2)cc1] | 297.1 | ¹H NMR (500 MHz, CDCl₃) δ 7.97-7.91 (m, 2H), 7.48- 7.42 (m, 3H), 2.15 (d, J = 1.2 Hz, 10H), 1.86-1.78 (m, 5H) |
| 1008 | 2-(pyridin-2-yl)- 5-(3- (trifluoromethyl) phenyl)-1,3,4- thiadiazole | 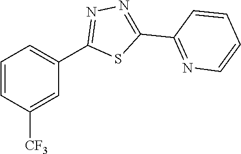 [SMILES: FC(F)(F)c1cccc(-c2nnc(-c3ccccn3)s2)c1] | 308.1 | ¹H NMR (500 MHz, CDCl₃) δ 8.69 (ddd, J = 4.9, 1.7, 1.0 Hz, 1H), 8.41 (dt, J = 7.9, 1.1 Hz, 1H), 8.34 (dp, J = 1.8, 0.8 Hz, 1H), 8.22 (dddd, J = 7.8, 1.8, 1.2, 0.6 Hz, 1H), 7.89 (td, J = 7.8, 1.7 Hz, 1H), 7.82-7.76 (m, 1H), 7.66 (tt, J = 7.8, 0.7 Hz, 1H), 7.43 (ddd, J = 7.6, 4.9, 1.2 Hz, 1H). |
| 1009 | 2-(pyridin-2-yl)- 5-(4- (trifluoromethyl) phenyl)-1,3,4- thiadiazole | 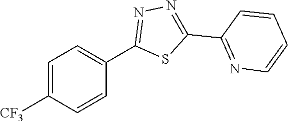 [SMILES: FC(F)(F)c1ccc(-c2nnc(-c3ccccn3)s2)cc1] | 308.1 | ¹H NMR (500 MHz, CDCl₃) δ 8.69 (ddd, J = 4.8, 1.6, 0.8 Hz, 1H), 8.41 (dt, J = 7.9, 1.1 Hz, 1H), 8.20-8.15 (m, 2H), 7.89 (td, J = 7.8, 1.8 Hz, 1H), 7.80-7.76 (m, 2H), 7.45-7.40 (m, 1H). |
| 1010 | 2-phenyl-5- (pyrazin-2-yl)- 1,3,4-thiadiazole | 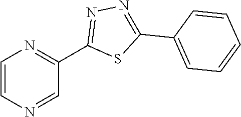 [SMILES: c1ccc(-c2nnc(-c3cnccn3)s2)cc1] | 241.1 | ¹H NMR (500 MHz, CDCl₃) δ 9.65 (d, J = 2.0 Hz, 1H), 8.71-8.65 (m, 2H), 8.09-8.06 (m, 2H), 7.56-7.53 (m, 3H). |
| 1011 | 2-(2- nitrophenyl)-5- (m-tolyl)-1,3,4- thiadiazole | 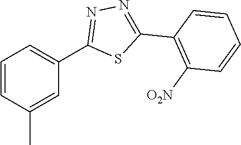 [SMILES: Cc1cccc(-c2nnc(-c3ccccc3[N+](=O)[O-])s2)c1] | 298.1 | ¹H NMR (500 MHz, CDCl₃) δ 8.64 (d, J = 4.7 Hz, 1H), 8.11 (d, J = 8.0 Hz, 1H), 7.91 (d, J = 7.8 Hz, 2H), 7.77- 7.69 (m, 1H), 7.30 (dd, J = 7.8, 4.8 Hz, 1H), 7.14 (d, J = 7.9 Hz, 2H), 2.23 (s, 3H). |
| 1012 | 2-(3- methoxyphenyl)- 5-phenyl-1,3,4- thiadiazole | 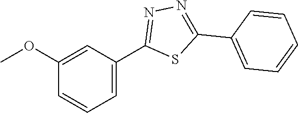 [SMILES: COc1cccc(-c2nnc(-c3ccccc3)s2)c1] | 269.1 | ¹H NMR (500 MHz, CDCl₃) δ 8.05-7.99 (m, 2H), 7.64 (dd, J = 2.6, 1.6 Hz, 1H), 7.55-7.48 (m, 4H), 7.40 (dd, J = 8.2, 7.7 Hz, 1H), 7.05 (ddd, J = 8.3, 2.6, 1.0 Hz, 1H), 3.91 (s, 3H). |
| 1013 | 2-(2- methoxyphenyl)- 5-phenyl-1,3,4- thiadiazole | 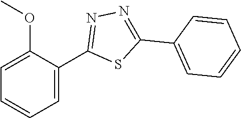 [SMILES: COc1ccccc1-c1nnc(-c2ccccc2)s1] | 269.1 | ¹H NMR (500 MHz, CDCl₃) δ 8.56 (dd, J = 7.9, 1.7 Hz, 1H), 8.10-8.04 (m, 2H), 7.53-7.46 (m, 4H), 7.18- 7.13 (m, 1H), 7.08 (dd, J = 8.4, 1.0 Hz, 1H), 4.06 (s, 3H). |
| 1014 | 2-phenyl-5-(3- (trifluoromethyl) phenyl)-1,3,4- thiadiazole | 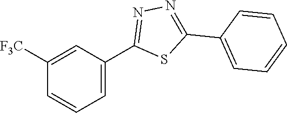 [SMILES: FC(F)(F)c1cccc(-c2nnc(-c3ccccc3)s2)c1] | 307.1 | ¹H NMR (500 MHz, CDCl₃) δ 8.28 (ddt, J = 2.5, 1.8, 0.7 Hz, 1H), 8.22 (dddd, J = 7.8, 1.7, 1.2, 0.6 Hz, 1H), 8.06- 8.00 (m, 2H), 7.79-7.74 (m, 1H), 7.65 (tt, J = 7.8, 0.7 Hz, 1H), 7.57-7.48 (m, 3H). |
| 1015 | 2-phenyl-5-(o- tolyl)-1,3,4- thiadiazole | 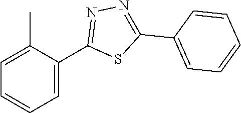 [SMILES: Cc1ccccc1-c1nnc(-c2ccccc2)s1] | 253 | ¹H NMR (500 MHz, CDCl₃) δ 8.26 (dq, J = 6.1, 1.7 Hz, 2H), 8.07-8.01 (m, 2H), 7.55 (d, J = 6.1 Hz, 3H), 7.35 (d, J = 7.7 Hz, 2H), 2.46 (s, 3H). |
| 1016 | 2-(4-(tert- butyl)phenyl)-5- (2-nitrophenyl)- 1,3,4-thiadiazole | 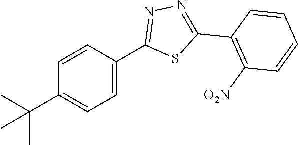 [SMILES: CC(C)(C)c1ccc(-c2nnc(-c3ccccc3[N+](=O)[O-])s2)cc1] | 340.1 | ¹H NMR (500 MHz, CDCl₃) δ 8.03 (dd, J = 8.1, 1.3 Hz, 1H), 7.97-7.93 (m, 2H), 7.84 (dd, J = 7.6, 1.5 Hz, 1H), 7.74 (td, J = 7.6, 1.4 Hz, 1H), 7.71-7.66 (m, 1H), 7.55-7.51 (m, 2H), 1.37 (s, 9H). |
| 1017 | 2-(4- chlorophenyl)-5- phenyl-1,3,4- thiadiazole | 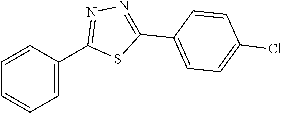 [SMILES: Clc1ccc(-c2nnc(-c3ccccc3)s2)cc1] | 273.1 | ¹H NMR (500 MHz, CDCl₃) δ 7.82-7.77 (m, 2H), 7.76- 7.72 (m, 2H), 7.32-7.24 (m, 5H). |
| 1018 | 2-(4- bromophenyl)-5- (4- methoxyphenyl)- 1,3,4-thiadiazole | 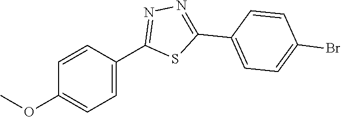 [SMILES: COc1ccc(-c2nnc(-c3ccc(Br)cc3)s2)cc1] | 348.1 | ¹H NMR (400 MHz, CDCl₃) δ 7.95 (dd, J = 8.9, 2.2 Hz, 2H), 7.87 (dt, J = 8.9, 2.5 Hz, 2H), 7.63 (dd, J = 8.8, 2.2 Hz, 2H), 7.01 (dd, J = 9.2, 2.2 Hz, 2H), 3.88 (dd, J = 2.7, 1.0 Hz, 3H). |
| 1019 | 2-(furan-2-yl)-5- (4- methoxyphenyl)- 1,3,4-thiadiazole | 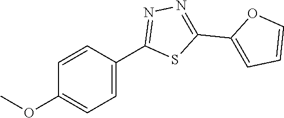 [SMILES: COc1ccc(-c2nnc(-c3ccco3)s2)cc1] | 259 | ¹H NMR (500 MHz, CDCl₃) δ 8.23-8.15 (m, 2H), 7.88- 7.84 (m, 1H), 7.45 (dd, J = 3.5, 0.7 Hz, 1H), 7.28-7.22 (m, 2H), 6.85 (dd, J = 3.5, 1.8 Hz, 1H), 4.13 (s, 3H). |
| 1020 | 2-(4- methoxyphenyl)- 5-(5- methylfuran-2- yl)-1,3,4- thiadiazole | 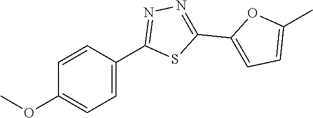 | 273.1 | ¹H NMR (400 MHz, CDCl₃) δ 7.92-7.88 (m, 2H), 7.05 (d, J = 3.4 Hz, 1H), 7.00- 6.94 (m, 3H), 6.16 (d, J = 3.4 Hz, 1H), 3.85 (d, J = 1.1 Hz, 3H), 2.40 (s, 3H). ¹³C NMR (100 MHz, CDCl₃) δ 166.40, 161.79, 157.68, 155.42, 143.99, 130.29, 129.35, 122.66, 114.85, 114.50, 114.46, 112.70, 108.84, 108.80, 55.45, 55.41, 13.83, 13.80. |
| 1021 | 2-(4- methoxyphenyl)- 5-(2- nitrophenyl)- 1,3,4-thiadiazole | 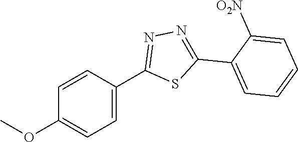 [SMILES: COc1ccc(-c2nnc(-c3ccccc3[N+](=O)[O-])s2)cc1] | 314.1 | ¹H NMR (500 MHz, CDCl₃) δ 8.29-8.23 (m, 1H), 8.22- 8.15 (m, 2H), 8.05 (ddq, J = 6.2, 3.7, 1.2 Hz, 1H), 7.98 (ddt, J = 4.9, 2.7, 1.3 Hz, 1H), 7.95-7.89 (m, 1H), 7.28-7.22 (m, 2H), 4.13 (dd, J = 3.8, 1.6 Hz, 3H). |
| 1022 | 2-(4- methoxyphenyl)- 5-(thiophen-2- yl)-1,3,4- thiadiazole | 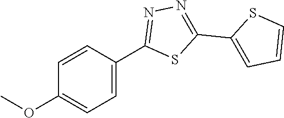 [SMILES: COc1ccc(-c2nnc(-c3cccs3)s2)cc1] | 275 | ¹H NMR (399 MHz, CDCl₃) δ 7.93 (dd, J = 9.0, 0.9 Hz, 2H), 7.58-7.55 (m, 1H), 7.49 (dt, J = 5.1, 1.0 Hz, 1H), 7.14 (ddd, J = 5.0, 3.7, 0.9 Hz, 1H), 7.02-6.97 (m, 2H), 3.88 (d, J = 0.9 Hz, 3H). |
| 1023 | 2-(3-methoxy-2- nitrophenyl)-5- phenyl-1,3,4- thiadiazole | 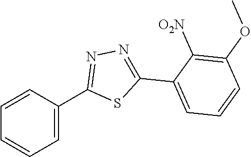 [SMILES: COc1cccc(-c2nnc(-c3ccccc3)s2)c1[N+](=O)[O-]] | 314 | ¹H NMR (400 MHz, CDCl₃) δ 8.10 (dt, J = 7.6, 1.4 Hz, 1H), 8.05 (dt, J = 7.9, 1.3 Hz, 1H), 7.98-7.94 (m, 2H), 7.78 (dtt, J = 22.0, 7.6, 1.4 Hz, 2H), 7.36-7.31 (m, 2H), 3.89 (s, 3H). |
| 1024 | 2-(2- chlorophenyl)-5- (4- methoxyphenyl)- 1,3,4-thiadiazole | 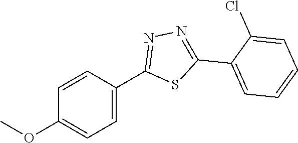 [SMILES: COc1ccc(-c2nnc(-c3ccccc3Cl)s2)cc1] | 303.1 | ¹H NMR (399 MHz, CDCl₃) δ 8.32 (dd, J = 6.1, 3.6 Hz, 1H), 7.96 (d, J = 8.8 Hz, 2H), 7.50 (dd, J = 5.9, 3.5 Hz, 1H), 7.39 (dd, J = 6.0, 3.5 Hz, 2H), 6.98 (d, J = 8.8 Hz, 2H), 3.85 (s, 3H). |
| 1025 | 2-(quinolin-4-yl)- 5-(p-tolyl)-1,3,4- thiadiazole | 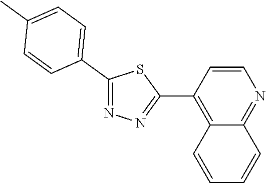 [SMILES: Cc1ccc(-c2nnc(-c3ccnc4ccccc34)s2)cc1] | 304.1 | ¹H NMR (399 MHz, CDCl₃) δ 9.04 (d, J = 4.4 Hz, 1H), 8.88 (d, J = 8.5 Hz, 1H), 8.22 (d, J = 8.4 Hz, 1H), 7.96 (dd, J = 8.1, 1.4 Hz, 2H), 7.82 (ddt, J = 8.3, 6.8, 1.4 Hz, 1H), 7.74 (dd, J = 4.4, 1.2 Hz, 1H), 7.72-7.67 (m, 1H), 7.34 (d, J = 7.8 Hz, 2H), 2.45 (s, 3H). |
| 1026 | 5-methoxy-2-(5- phenyl-1,3,4- thiadiazol-2- yl)aniline | 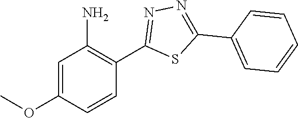 [SMILES: COc1ccc(-c2nnc(-c3ccccc3)s2)c(N)c1] | 28 | ¹H NMR (400 MHz, CDCl₃) δ 8.18-8.12 (m, 2H), 7.78 (d, J = 7.8 Hz, 1H), 7.58- 7.52 (m, 3H), 7.21 (d, J = 7.1 Hz, 1H), 6.75 (t, J = 7.6 Hz, 1H), 5.97 (br s, 2H), 3.56 (s, 3H). |
| 1027 | 2-(5-(4-(tert- butyl)phenyl)- 1,3,4-thiadiazol- 2-yl)aniline | 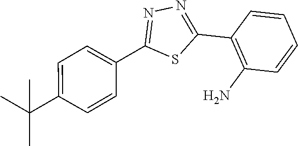 [SMILES: CC(C)(C)c1ccc(-c2nnc(-c3ccccc3N)s2)cc1] | 310.1 | ¹H NMR (500 MHz, CDCl₃) δ 7.97-7.91 (m, 2H), 7.57- 7.49 (m, 3H), 7.22 (ddd, J = 8.4, 7.1, 1.5 Hz, 1H), 6.80 (dd, J = 8.2, 1.1 Hz, 1H), 6.74 (ddd, J = 8.1, 7.2, 1.2 Hz, 1H), 1.37 (s, 9H). |
| 1028 | 2-(2- nitrobenzyl)-5- (4- (trifluoromethyl) phenyl)-1,3,4- thiadiazole | 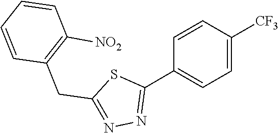 [SMILES: O=[N+]([O-])c1ccccc1Cc1nnc(-c2ccc(C(F)(F)F)cc2)s1] | 366.1 | ¹H NMR (500 MHz, CDCl₃) δ 8.10 (dd, J = 8.2, 1.3 Hz, 1H), 8.06-8.01 (m, 2H), 7.71 (d, J = 8.2 Hz, 2H), 7.68- 7.58 (m, 2H), 7.50 (ddd, J = 8.4, 7.0, 1.8 Hz, 1H), 4.81 (s, 2H). |
| 1029 | 2-(5- methylfuran-2- yl)-5-(p-tolyl)- 1,3,4-thiadiazole | 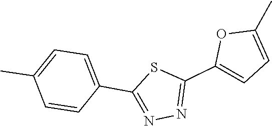 | 257.1 | ¹H NMR (500 MHz, CDCl₃) δ 7.90-7.83 (m, 2H), 7.31- 7.27 (m, 2H), 7.08 (dd, J = 3.3, 0.6 Hz, 1H), 6.19 (dq, J = 3.2, 1.0 Hz, 1H), 2.42 (d, J = 0.7 Hz, 6H). |
| 1030 | 4-(5-phenyl- 1,3,4-thiadiazol- 2-yl)aniline | 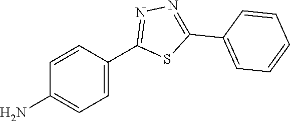 [SMILES: Nc1ccc(-c2nnc(-c3ccccc3)s2)cc1] | 254 | ¹H NMR (400 MHz, CDCl₃) 7.99 (ddt, J = 4.8, 2.4, 1.0 Hz, 2H), 7.82 (dd, J = 8.7, 0.8 Hz, 2H), 7.51-7.45 (m, 3H), 6.76-6.71 (m, 2H), 4.01 (s, 2H). |
| 1031 | 2-(thiophen-2- yl)-5-(p-tolyl)- 1,3,4-thiadiazole | 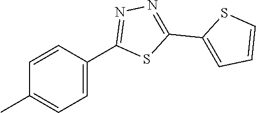 [SMILES: Cc1ccc(-c2nnc(-c3cccs3)s2)cc1] | 259.1 | ¹H NMR (399 MHz, CDCl₃) δ 7.87 (d, J = 8.0 Hz, 2H), 7.58 (dt, J = 3.7, 1.0 Hz, 1H), 7.51-7.47 (m, 1H), 7.29 (d, J = 7.9 Hz, 2H), 7.14 (ddd, J = 5.2, 3.7, 0.8 Hz, 1H), 2.42 (s, 3H). |
| 1032 | N-(5-phenyl- 1,3,4-thiadiazol- 2-yl)benzamide | 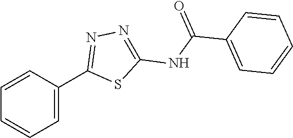 [SMILES: O=C(Nc1nnc(-c2ccccc2)s1)c1ccccc1] | 282.1 | ¹H NMR (500 MHz, CDCl₃) δ 8.13-8.02 (m, 4H), 7.57 (dt, J = 18.7, 6.8 Hz, 6H). |
| 1033 | N-(5-(4- methoxyphenyl)- 1,3,4-thiadiazol- 2-yl)benzamide | 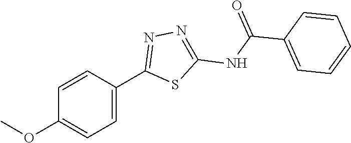 [SMILES: COc1ccc(-c2nnc(NC(=O)c3ccccc3)s2)cc1] | 312.1 | ¹H NMR (399 MHz, dmso) δ 7.97-7.91 (m, 4H), 7.48- 7.41 (m, 4H), 7.22 (d, J = 8.0 Hz, 2H), 3.89 (s, 3H). |
| 1034 | N-(5-(2- chlorophenyl)- 1,3,4-thiadiazol- 2-yl)-4- methylbenzamide | 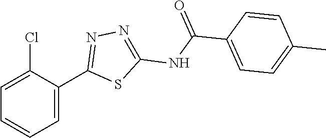 [SMILES: Cc1ccc(C(=O)Nc2nnc(-c3ccccc3Cl)s2)cc1] | 330.1 | ¹H NMR (399 MHz, dmso) δ 7.97-7.91 (m, 4H), 7.48- 7.41 (m, 4H), 7.22 (d, J = 8.0 Hz, 1H), 2.35 (s, 3H). |
| 1035 | 2-(5-methoxy-2- nitrophenyl)-5- phenyl-1,3,4- thiadiazole | 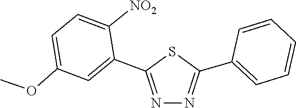 [SMILES: COc1ccc([N+](=O)[O-])c(-c2nnc(-c3ccccc3)s2)c1] | 314 | ¹H NMR (400 MHz, CDCl₃) δ 8.18 (d, J = 9.1 Hz, 1H), 8.05-8.02 (m, 2H), 7.57- 7.51 (m, 3H), 7.27 (d, J = 2.8 Hz, 1H), 7.16-7.12 (m, 1H), 3.97 (s, 3H). |
| 1036 | 2-(4-methoxy-2- nitrophenyl)-5- phenyl-1,3,4- thiadiazole | 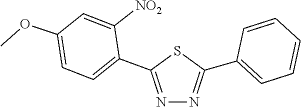 [SMILES: COc1ccc(-c2nnc(-c3ccccc3)s2)c([N+](=O)[O-])c1] | 314 | ¹H NMR (400 MHz, CDCl₃) δ 8.04-8.01 (m, 2H), 7.79 (d, J = 8.6 Hz, 1H), 7.55- 7.51 (m, 4H), 7.27-7.24 (m, 1H), 3.98 (s, 3H). |
| 1037 | 2-(4-bromo-2- nitrophenyl)-5- phenyl-1,3,4- thiadiazole | 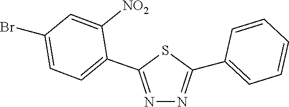 [SMILES: O=[N+]([O-])c1cc(Br)ccc1-c1nnc(-c2ccccc2)s1] | 363 | ¹H NMR (400 MHz, CDCl₃) δ 8.18 (d, J = 1.9 Hz, 1H), 8.04-8.01 (m, 2H), 7.92- 7.87 (m, 1H), 7.76 (d, J = 8.2 Hz, 1H), 7.56-7.51 (m, 3H). |
| 1038 | 2-benzyl-5- phenyl-1,3,4- thiadiazole | 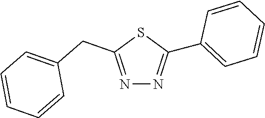 [SMILES: c1ccc(Cc2nnc(-c3ccccc3)s2)cc1] | 253.1 | |
| 1039 | 2-([1,1′- biphenyl]-4- ylmethyl)-5- phenyl-1,3,4- thiadiazole | 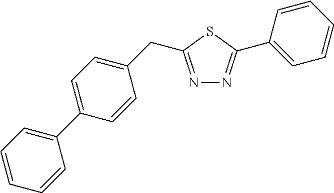 [SMILES: c1ccc(-c2ccc(Cc3nnc(-c4ccccc4)s3)cc2)cc1] | 329.1 | |
| 1040 | 2-(2- chlorophenyl)-5- (o-tolyl)-1,3,4- thiadiazole | 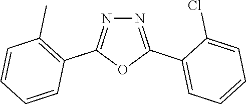 [SMILES: Cc1ccccc1-c1nnc(-c2ccccc2Cl)o1] | 314.1 | ¹H NMR (399 MHz, CDCl₃) δ 8.41 (dd, J = 6.0, 3.6 Hz, 1H), 7.77 (d, J = 7.7 Hz, 1H), 7.56 (dd, J = 5.9, 3.5 Hz, 1H), 7.48-7.42 (m, 2H), 7.42-7.30 (m, 3H), 2.66 (s, 3H). |
| 1041 | 2-(2- chlorophenyl)-5- (m-tolyl)-1,3,4- thiadiazole | 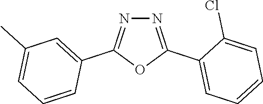 [SMILES: Cc1cccc(-c2nnc(-c3ccccc3Cl)o2)c1] | 314.1 | ¹H NMR (399 MHz, dmso) δ 8.23 (dt, J = 7.9, 2.1 Hz, 1H), 7.98-7.85 (m, 2H), 7.74 (dt, J = 8.7, 1.7 Hz, 1H), 7.66- 7.56 (m, 2H), 7.50-7.38 (m, 2H), 2.42 (s, 3H). ¹³C NMR (100 MHz, dmso) δ 169.23, 162.87, 139.08, 132.52, 132.26, 131.54, 131.03, 130.76, 129.46, 129.20, 128.23, 128.06, 124.88, 20.80. |
| 1042 | 2-(quinolin-2-yl)- 5-(p-tolyl)-1,3,4- thiadiazole | 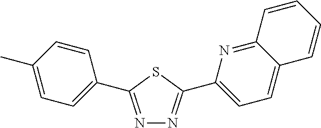 [SMILES: Cc1ccc(-c2nnc(-c3ccc4ccccc4n3)s2)cc1] | 304.1 | ¹H NMR (500 MHz, CDCl₃) δ 9.31 (d, J = 8.5 Hz, 1H), 9.10 (d, J = 4.5 Hz, 1H), 8.23 (d, J = 8.4 Hz, 1H), 8.16 (d, J = 8.5 Hz, 2H), 8.08 (d, J = 4.4 Hz, 1H), 7.85 (ddd, J = 8.4, 6.9, 1.5 Hz, 1H), 7.79- 7.73 (m, 1H), 7.08 (d, J = 8.6 Hz, 2H), 2.46 (s, 3H). |
| 1043 | 2-(naphthalen-1- yl)-5-(p-tolyl)- 1,3,4-thiadiazole | 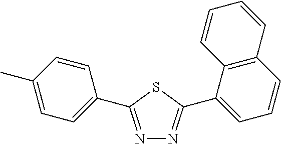 [SMILES: Cc1ccc(-c2nnc(-c3cccc4ccccc34)s2)cc1] | 303.1 | ¹H NMR (399 MHz, dmso) δ 8.70 (d, J = 8.1 Hz, 1H), 8.19 (d, J = 8.3 Hz, 1H), 8.10 (d, J = 7.7 Hz, 1H), 8.01- 7.96 (m, 3H), 7.69 (q, J = 7.1 Hz, 3H), 7.43 (d, J = 7.8 Hz, 2H), 2.42 (s, 3H). |
| 1044 | 2-(naphthalen-2- yl)-5-(p-tolyl)- 1,3,4-thiadiazole | 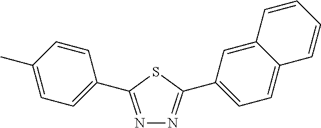 [SMILES: Cc1ccc(-c2nnc(-c3ccc4ccccc4c3)s2)cc1] | 303.1 | ¹H NMR (399 MHz, dmso) δ 8.60 (s, 1H), 8.17-8.08 (m, 3H), 8.04-7.99 (m, 1H), 7.93 (dd, J = 8.3, 2.3 Hz, 2H), 7.63 (dt, J = 4.7, 2.3 Hz, 2H), 7.40 (dd, J = 8.1, 2.4 Hz, 2H), 2.40 (d, J = 2.2 Hz, 3H). |
| 1045 | 2-(3- chlorophenyl)-5- (p-tolyl)-1,3,4- thiadiazole | 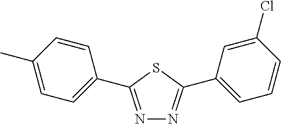 [SMILES: Cc1ccc(-c2nnc(-c3cccc(Cl)c3)s2)cc1] | 287.1 | ¹H NMR (399 MHz, CDCl₃) δ 7.41 (t, J = 1.7 Hz, 1H), 7.29 (d, J = 8.0 Hz, 3H), 6.88- 6.80 (m, 1H), 6.69 (dd, J = 8.0, 4.3 Hz, 3H), 1.83-1.82 (m, 3H). |
| 1046 | 4-(5-(naphthalen- 2-yl)-1,3,4- thiadiazol-2- yl)phenol | 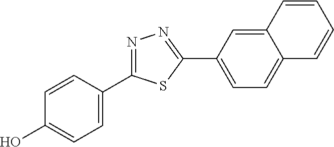 [SMILES: Oc1ccc(-c2nnc(-c3ccc4ccccc4c3)s2)cc1] | 305.1 | ¹H NMR (500 MHz, CDCl₃) δ 8.390-8.21 (m, 5H), 7.91- 7.76 (m, 2H), 7.63 (t, J = 7.5 Hz, 1H), 7.56 (t, J = 7.6 Hz, 3H). |
| 1047 | 4-(5-(quinolin-2- yl)-1,3,4- thiadiazol-2- yl)phenol | 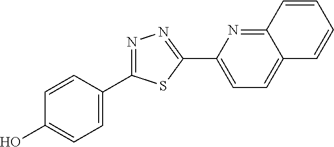 [SMILES: Oc1ccc(-c2nnc(-c3ccc4ccccc4n3)s2)cc1] | 306.1 | ¹H NMR (399 MHz, CDCl₃) δ 8.61 (s, 1H), 8.21 (d, J = 8.8 Hz, 1H), 8.10 (d, J = 8.0 Hz, 2H), 7.98 (dd, J = 8.3 Hz, 1H), 7.90 (s, 1H), 7.62- 7.55 (m, 2H), 7.01 (d, J = 8.3 Hz, 2H). |
| 1048 | 2-(4- chlorophenyl)-5- (p-tolyl)-1,3,4- thiadiazole | 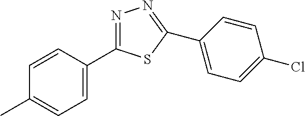 [SMILES: Cc1ccc(-c2nnc(-c3ccc(Cl)cc3)s2)cc1] | 287.1 | ¹H NMR (399 MHz, CDCl₃) δ 7.96-7.92 (m, 2H), 7.91- 7.87 (m, 2H), 7.49-7.45 (m, 2H), 7.30 (d, J = 8.0 Hz, 2H), 2.43 (s, 3H). |
| 1049 | methyl 2-(5-(2- chlorophenyl)- 1,3,4-thiadiazol- 2-yl)benzoate | 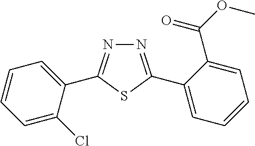 [SMILES: COC(=O)c1ccccc1-c1nnc(-c2ccccc2Cl)s1] | 331.1 | ¹H NMR (400 MHz, CDCl₃) δ 8.09 (dt, J = 7.5, 1.3 Hz, 1H), 8.06-7.95 (m, 3H), 7.84-7.70 (m, 2H), 7.06- 6.97 (m, 2H), 3.89 (s, 3H). |
| 1050 | methyl 3-(5-(2- chlorophenyl)- 1,3,4-thiadiazol- 2-yl)benzoate | 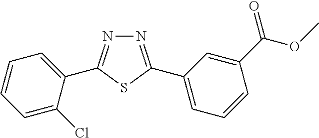 [SMILES: COC(=O)c1cccc(-c2nnc(-c3ccccc3Cl)s2)c1] | 331.1 | ¹H NMR (399 MHz, CDCl₃) δ 8.64 (q, J = 1.5 Hz, 1H), 8.38 (dt, J = 7.8, 3.2 Hz, 1H), 8.31 (dt, J = 7.8, 1.5 Hz, 1H), 8.17 (dt, J = 7.8, 1.4 Hz, 1H), 7.60 (t, J = 7.8 Hz, 1H), 7.57- 7.53 (m, 1H), 7.47-7.40 (m, 2H), 3.97 (d, J = 1.0 Hz, 3H). |
| 1051 | methyl 4-(5-(2- chlorophenyl)- 1,3,4-thiadiazol- 2-yl)benzoate | 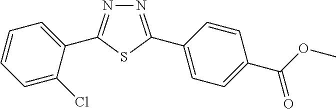 [SMILES: COC(=O)c1ccc(-c2nnc(-c3ccccc3Cl)s2)cc1] | 331.1 | ¹H NMR (399 MHz, CDCl₃) δ 8.24-8.19 (m, 1H), 8.00- 7.93 (m, 4H), 7.40-7.35 (m, 1H), 7.30-7.24 (m, 2H), 3.78 (d, J = 0.9 Hz, 3H). |
| 1052 | 2-(4- butoxyphenyl)-5- (2-chlorophenyl)- 1,3,4-thiadiazole | 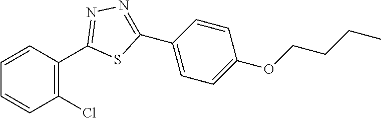 [SMILES: CCCCOc1ccc(-c2nnc(-c3ccccc3Cl)s2)cc1] | 345.1 | ¹H NMR (399 MHz, dmso) δ 8.20 (d, J = 7.6 Hz, 1H), 7.99 (dd, J = 8.7, 1.8 Hz, 2H), 7.73 (d, J = 7.8 Hz, 1H), 7.60 (dt, J = 16.1, 7.4 Hz, 2H), 7.12 (d, J = 8.4 Hz, 2H), 4.07 (t, J = 6.4 Hz, 2H), 1.73 (p, J = 6.9 Hz, 2H), 1.45 (h, J = 7.4 Hz, 2H), 0.94 (dd, J= 8.3, 6.7 Hz, 3H). ¹³C NMR (100 MHz, dmso) δ 168.97, 158.14, 142.50, 129.47, 128.38, 115.41, 67.62, 40.15, 39.94, 39.73, 39.60, 39.52, 39.31, 39.10, 38.89, 30.65, 18.73, 13.73. |
| 1053 | 2-(1-phenyl-1H- pyrazol-4-yl)-5- (p-tolyl)-1,3,4- thiadiazole | 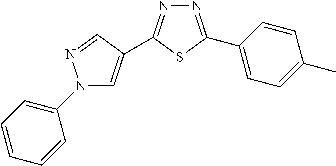 [SMILES: Cc1ccc(-c2nnc(-c3cnn(-c4ccccc4)c3)s2)cc1] | 319.1 | ¹H NMR (399 MHz, dmso) δ 9.31 (s, 1H), 8.38 (s, 1H), 7.96 (d, J = 8.0 Hz, 2H), 7.89 (d, J = 7.8 Hz, 2H), 7.56 (t, J = 7.7 Hz, 2H), 7.39 (d, J = 7.6 Hz, 3H), 2.39 (s, 3H). |
| 1054 | (4-(5-(2- chlorophenyl)- 1,3,4-thiadiazol- 2- yl)phenyl) boronic acid | 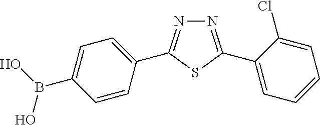 [SMILES: OB(O)c1ccc(-c2nnc(-c3ccccc3Cl)s2)cc1] | 317.1 | ¹H NMR (500 MHz, CDCl₃) δ 8.01 (dt, J = 7.7, 1.5 Hz, 1H), 7.69 (tdd, J = 8.8, 7.7, 1.2 Hz, 4H), 7.49 (dt, J = 8.0, 1.3 Hz, 1H), 7.41 (tt, J = 7.9, 1.5 Hz, 1H), 7.22 (ddt, J = 8.8, 7.5, 1.3 Hz, 1H). |
| 1055 | (4-(5-(p-tolyl)- 1,3,4-thiadiazol- 2- yl)phenyl) boronic acid | 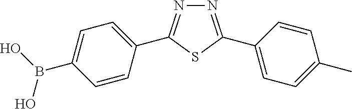 [SMILES: Cc1ccc(-c2nnc(-c3ccc(B(O)O)cc3)s2)cc1] | 297.1 | ¹H NMR (500 MHz, CDCl₃) δ 8.01 (q, J = 3.7 Hz, 2H), 7.93-7.86 (m, 2H), 7.50 (q, J = 3.5 Hz, 3H), 7.30 (d, J = 7.0 Hz, 2H), 2.43 (d, J = 3.3 Hz, 3H). |
| 1056 | (4-(5-(4- methoxyphenyl)- 1,3,4-thiadiazol- 2- yl)phenyl) boronic acid | 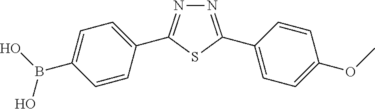 [SMILES: COc1ccc(-c2nnc(-c3ccc(B(O)O)cc3)s2)cc1] | 313.1 | ¹H NMR (500 MHz, CDCl₃) δ 8.13-8.05 (m, 3H), 7.58 (dt, J = 8.1, 1.2 Hz, 1H), 7.51- 7.40 (m, 2H), 7.09-7.01 (m, 2H), 3.90 (d, J = 1.0 Hz, 3H). |
| 1057 | 2-(2- chlorophenyl)-5- (1-phenyl-1H- pyrazol-4-yl)- 1,3,4-thiadiazole | 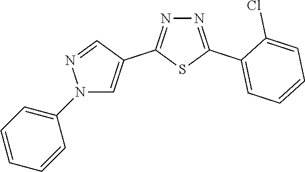 [SMILES: Clc1ccccc1-c1nnc(-c2cnn(-c3ccccc3)c2)s1] | 339.1 | |
| 1058 | 2-(quinolin-5-yl)- 5-(p-tolyl)-1,3,4- thiadiazole | 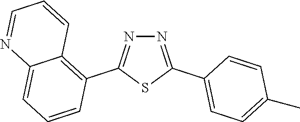 [SMILES: Cc1ccc(-c2nnc(-c3cccc4ncccc34)s2)cc1] | 304.1 | ¹H NMR (399 MHz, CDCl₃) δ 9.25-9.18 (m, 1H), 8.74 (dq, J = 6.2, 1.1 Hz, 1H), 8.59 (dd, J = 6.2, 2.5 Hz, 1H), 8.02-7.92 (m, 2H), 7.84-7.79 (m, 2H), 7.55 (td, J = 8.0, 3.9 Hz, 1H), 7.20 (dd, J = 8.3, 3.2 Hz, 2H), 2.33 (d, J = 2.8 Hz, 3H). ¹³C NMR (100 MHz, CDCl₃) 8 168.78, 165.70, 152.86, 144.93, 141.86, 133.50, 132.93, 132.92, 130.83, 129.88, 128.85, 127.86, 126.96, 126.50, 125.94, 125.92, 118.62, 77.38, 21.52, 21.50. |
| 1059 | 2-(2- chlorophenyl)-5- (quinolin-5-yl)- 1,3,4-thiadiazole | 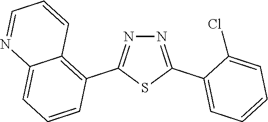 [SMILES: Clc1ccccc1-c1nnc(-c2cccc3ncccc23)s1] | 324 | ¹H NMR (399 MHz, CDCl₃) δ 9.33 (d, J = 2.6 Hz, 1H), 8.76 (dd, J = 6.2, 2.5 Hz, 1H), 8.66 (dd, J = 6.2, 2.6 Hz, 1H), 8.44 (dt, J = 6.5, 3.2 Hz, 1H), 8.12 (dt, J = 7.0, 3.0 Hz, 2H), 7.69 (td, J = 7.7, 2.5 Hz, 1H), 7.55 (dt, J = 6.5, 3.1 Hz, 1H), 7.45 (dq, J = 6.5, 3.5 Hz, 2H). ¹³C NMR (100 MHz, CDCl₃) δ 167.57, 164.04, 152.96, 145.07, 133.60, 133.18, 132.69, 131.86, 131.08, 131.05, 130.67, 128.97, 128.75, 127.49, 126.58, 125.91, 118.55, 77.25, 29.68, 1.01. |
| 1060 | 2-(thiazol-2-yl)- 5-(p-tolyl)-1,3,4- thiadiazole | 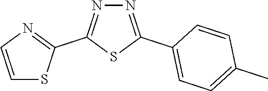 [SMILES: Cc1ccc(-c2nnc(-c3nccs3)s2)cc1] | 260.1 | ¹H NMR (399 MHz, CDCl₃) δ 8.91 (d, J = 3.1 Hz, 1H), 8.32 (d, J = 3.1 Hz, 1H), 7.89- 7.83 (m, 2H), 7.27 (m, 1H), 2.42 (d, J = 3.2 Hz, 3H). ¹³C NMR (100 MHz, CDCl₃) δ 168.42, 158.12, 155.46, 144.44, 142.14, 129.97, 128.60, 127.96, 126.81, 77.23, 21.55. |
| 1061 | 2-(2- chlorophenyl)-5- (thiazol-2-yl)- 1,3,4-thiadiazole | 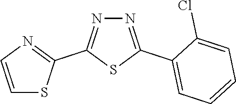 [SMILES: Clc1ccccc1-c1nnc(-c2nccs2)s1] | 280.1 | ¹H NMR (400 MHz, CDCl₃) δ 8.31-8.24 (m, 2H), 7.85- 7.79 (m, 2H), 7.74-7.69 (m, 1H), 7.31 (dt, J = 8.6, 4.2 Hz, 1H), 6.67 (ddp, J = 5.8, 4.1, 1.9 Hz, 1H). |
| 1062 | 2-(quinolin-5-yl)- 5-(4- (trifluoromethyl) phenyl)-1,3,4- thiadiazole | 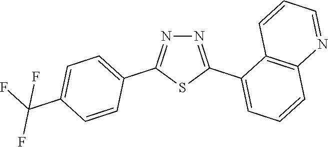 [SMILES: FC(F)(F)c1ccc(-c2nnc(-c3cccc4ncccc34)s2)cc1] | 358.1 | ¹H NMR (399 MHz, CDCl₃) δ 9.27 (d, J = 2.4 Hz, 1H), 8.73 (dd, J = 6.2, 2.3 Hz, 1H), 8.62 (dd, J = 6.2, 2.3 Hz, 1H), 8.13-7.99 (m, 4H), 7.75-7.68 (m, 2H), 7.62 (td, J = 7.8, 2.3 Hz, 1H). ¹³C NMR (100 MHz, CDCl₃) δ 167.03, 152.97, 145.15, 133.77, 133.02, 132.93, 132.90, 132.70, 131.33, 128.86, 128.23, 126.52, 126.27, 126.23, 126.19, 126.16, 125.54, 124.93, 122.22, 118.38, 77.30. |
| 1063 | 2-(thiazol-2-yl)- 5-(4- (trifluoromethyl) phenyl)-1,3,4- thiadiazole | 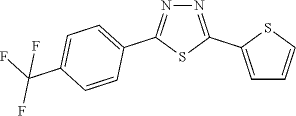 [SMILES: FC(F)(F)c1ccc(-c2nnc(-c3cccs3)s2)cc1] | 314.1 | ¹H NMR (399 MHz, CDCl₃) δ 8.97 (s, 1H), 8.39 (s, 1H), 8.13 (d, J = 8.1 Hz, 2H), 7.79 (d, J = 8.1 Hz, 2H). |
| 1064 | methyl 3-(5-(4- (trifluoromethyl) phenyl)-1,3,4- thiadiazol-2- yl)benzoate | 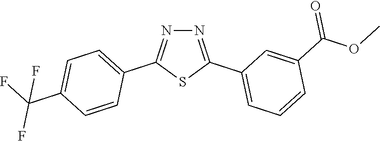 [SMILES: COC(=O)c1cccc(-c2nnc(-c3ccc(C(F)(F)F)cc3)s2)c1] | 365.1 | ¹H NMR (500 MHz, CDCl₃) δ 8.11-8.03 (m, 3H), 7.58 (dt, J = 8.1, 1.2 Hz, 1H), 7.51- 7.40 (m, 2H), 7.09-7.01 (m, 2H), 3.80 (d, J = 1.0 Hz, 3H). |
| 1065 | methyl 2-(5-(4- (trifluoromethyl) phenyl)-1,3,4- thiadiazol-2- yl)benzoate | 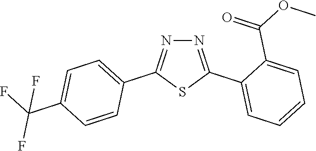 [SMILES: COC(=O)c1ccccc1-c1nnc(-c2ccc(C(F)(F)F)cc2)s1] | 365.1 | ¹H NMR (500 MHz, CDCl₃) δ 8.21 (d, J = 151.9 Hz, 6H), 7.02 (s, 2H), 3.94 (d, J = 69.7 Hz, 3H). |
| 1066 | methyl 4-(5-(4- (trifluoromethyl) phenyl)-1,3,4- thiadiazol-2- yl)benzoate | 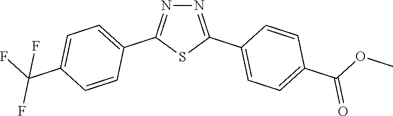 [SMILES: COC(=O)c1ccc(-c2nnc(-c3ccc(C(F)(F)F)cc3)s2)cc1] | 365.1 | ¹H NMR (399 MHz, CDCl₃) δ 8.17 (dd, J = 11.4, 8.2 Hz, 4H), 8.11 (d, J = 8.1 Hz, 2H), 7.79 (d, J = 8.1 Hz, 2H), 3.97 (s, 3H). |
| 1067 | (4-(5-(4- (trifluoromethyl) phenyl)-1,3,4- thiadiazol-2- yl)phenyl) boronic acid | 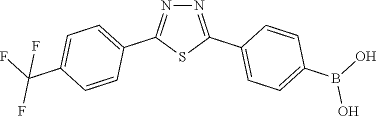 [SMILES: OB(O)c1ccc(-c2nnc(-c3ccc(C(F)(F)F)cc3)s2)cc1] | 351 | ¹H NMR (400 MHz, CDCl₃) δ 8.20 (t, J = 7.4 Hz, 2H), 8.11-8.03 (m, 2H), 7.88- 7.78 (m, 4H). |
| 1068 | (4-(5-(4-(tert- butyl)phenyl)- 1,3,4-thiadiazol- 2- yl)phenyl) boronic acid | 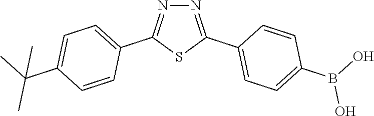 [SMILES: CC(C)(C)c1ccc(-c2nnc(-c3ccc(B(O)O)cc3)s2)cc1] | 339.1 | |
| 1069 | 2-(6- methoxypyridin- 2-yl)-5-(3- (trifluoromethyl) phenyl)-1,3,4- thiadiazole | 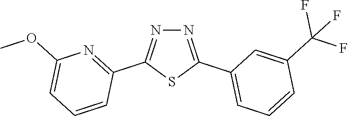 [SMILES: COc1cccc(-c2nnc(-c3cccc(C(F)(F)F)c3)s2)n1] | 338.1 | |
| 1070 | 2-(6- methoxypyridin- 3-yl)-5-(3- (trifluoromethyl) phenyl)-1,3,4- thiadiazole | 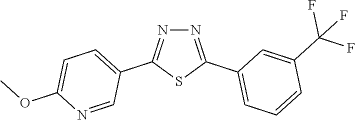 [SMILES: COc1ccc(-c2nnc(-c3cccc(C(F)(F)F)c3)s2)cn1] | 338.1 | |
| 1071 | 2-(6- methoxypyridin- 3-yl)-5-(4- (trifluoromethyl) phenyl)-1,3,4- thiadiazole | 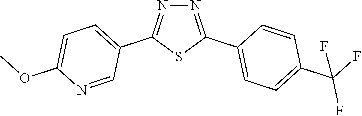 [SMILES: COc1ccc(-c2nnc(-c3ccc(C(F)(F)F)cc3)s2)cn1] | 338.1 | |
| 1072 | 2-(6- methylpyridin-2- yl)-5-(4- (trifluoromethyl) phenyl)-1,3,4- thiadiazole | 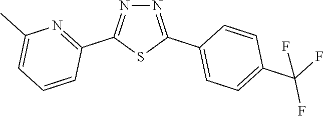 [SMILES: Cc1cccc(-c2nnc(-c3ccc(C(F)(F)F)cc3)s2)n1] | 322 | ¹H NMR (400 MHz, CDCl₃) δ 8.01-7.93 (m, 2H), 7.81 (dd, J = 3.7, 1.2 Hz, 1H), 7.54 (dt, J = 4.9, 1.0 Hz, 1H), 7.35-7.32 (m, 2H), 7.20 (ddd, J = 4.4, 3.6, 0.7 Hz, 1H), 2.45 (s, 3H). |
| 1073 | 2-(6- methylpyridin-2- yl)-5-(3- (trifluoromethyl) phenyl)-1,3,4- thiadiazole | 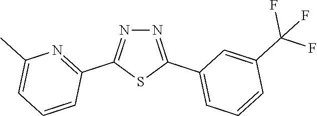 [SMILES: Cc1cccc(-c2nnc(-c3cccc(C(F)(F)F)c3)s2)n1] | 322 | ¹H NMR (500 MHz, CDCl₃) δ 8.44-8.39 (m, 2H), 8.35 (d, J = 8.9 Hz, 2H), 8.06 (d, J = 8.0 Hz, 2H), 7.38 (d, J = 7.9 Hz, 1H), 2.48 (s, 3H). |
| 1074 | 2-(2- chloropyridin-3- yl)-5-(3- (trifluoromethyl) phenyl)-1,3,4- thiadiazole | 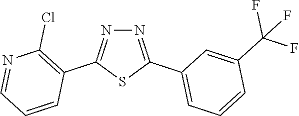 [SMILES: FC(F)(F)c1cccc(-c2nnc(-c3cccnc3Cl)s2)c1] | 342.1 | ¹H NMR (400 MHz, CDCl₃) δ 8.41-8.38 (m, 1H), 8.35 (dd, J = 7.8, 1.5 Hz, 1H), 7.85-7.81 (m, 1H), 7.72- 7.67 (m, 2H), 7.32-7.29 (m, 1H), 6.66 (dd, J = 3.5, 1.7 Hz, 1H). |
| 1075 | 2-(2- chloropyridin-3- yl)-5-(4- (trifluoromethyl) phenyl)-1,3,4- thiadiazole | 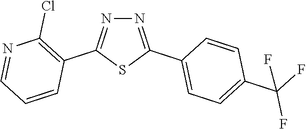 [SMILES: FC(F)(F)c1ccc(-c2nnc(-c3cccnc3Cl)s2)cc1] | 342.1 | |
| 1076 | 2-(1H-imidazol- 2-yl)-5-(p-tolyl)- 1,3,4-thiadiazole | 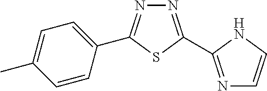 [SMILES: Cc1ccc(-c2nnc(-c3ncc[nH]3)s2)cc1] | 243.1 | ¹H NMR (399 MHz, CDCl₃) δ 9.00 (d, J = 3.0 Hz, 1H), 8.58 (d, J = 3.0 Hz, 1H), 8.05- 7.97 (m, 2H), 7.38-7.31 (m, 2H), 6.67 (d, 1H), 2.45 (d, J = 2.8 Hz, 3H). |
| 1077 | 2-(1-methyl-1H- imidazol-2-yl)-5- (p-tolyl)-1,3,4- thiadiazole | 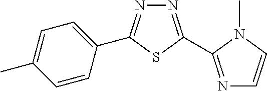 [SMILES: Cc1ccc(-c2nnc(-c3nccn3C)s2)cc1] | 257 | |
| 1078 | 2-(1H-imidazol- 4-yl)-5-(p-tolyl)- 1,3,4-thiadiazole | 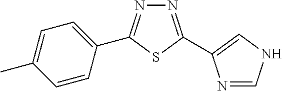 [SMILES: Cc1ccc(-c2nnc(-c3c[nH]cn3)s2)cc1] | 243.1 | ¹H NMR (500 MHz, CDCl₃) δ 8.02 (d, J = 8.0 Hz, 2H), 7.68 (d, J = 1.8 Hz, 1H), 7.34 (d, J = 7.9 Hz, 2H), 7.23 (d, J = 3.5 Hz, 1H), 6.63 (dd, J = 3.5, 1.7 Hz, 1H), 2.45 (s, 3H). |
| 1079 | 2-(4- methylthiazol-2- yl)-5-(p-tolyl)- 1,3,4-thiadiazole | 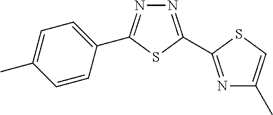 | 274 | ¹H NMR (500 MHz, CDCl₃) δ 8.02 (d, J = 8.0 Hz, 2H), 7.68 (d, J = 1.8 Hz, 1H), 7.34 (d, J = 7.9 Hz, 2H), 7.23 (d, J = 3.5 Hz, 1H), 6.63 (dd, J = 3.5, 1.7 Hz, 1H), 2.45 (s, 3H). |
| 1080 | 2-(4- methylthiazol-5- yl)-5-(p-tolyl)- 1,3,4-thiadiazole | 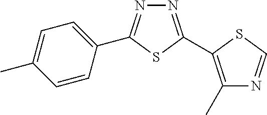 [SMILES: Cc1ccc(-c2nnc(-c3scnc3C)s2)cc1] | 274 | ¹H NMR (500 MHz, CDCl₃) δ 8.09-8.00 (m, 2H), 7.08 (d, J = 3.4 Hz, 1H), 7.04- 6.98 (m, 2H), 3.28 (d, J = 2.2 Hz, 3H), 2.34 (s, 3H). |
| 1081 | 4-(5-(p-tolyl)- 1,3,4-thiadiazol- 2-yl)oxazole | 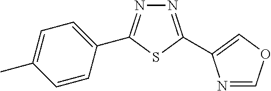 [SMILES: Cc1ccc(-c2nnc(-c3cocn3)s2)cc1] | 244 | |
| 1082 | 2-(thiazol-5-yl)- 5-(p-tolyl)-1,3,4- thiadiazole | 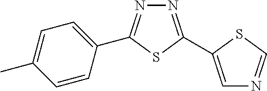 [SMILES: Cc1ccc(-c2nnc(-c3cncs3)s2)cc1] | 260 | ¹H NMR (399 MHz, CDCl₃) δ 8.95 (s, 1H), 8.33 (s, 1H), 7.86 (d, J = 7.9 Hz, 2H), 7.29 (d, J = 7.8 Hz, 2H), 2.42 (s, 3H). ¹³C NMR (100 MHz, CDCl₃) δ 168.53, 158.03, 155.69, 144.17, 142.19, 129.98, 128.66, 127.96, 126.72, 29.69, 21.56, 1.01. |
| 1083 | 2-(pyrimidin-5- yl)-5-(p-tolyl)- 1,3,4-thiadiazole | 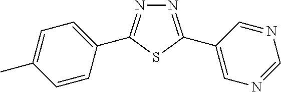 [SMILES: Cc1ccc(-c2nnc(-c3cncnc3)s2)cc1] | 255 | |
| 1084 | 2-(4-methyl-1H- imidazol-5-yl)-5- (p-tolyl)-1,3,4- thiadiazole | 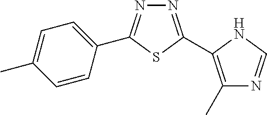 [SMILES: Cc1ccc(-c2nnc(-c3[nH]cnc3C)s2)cc1] | 257 | ¹H NMR (500 MHz, CDCl₃) δ 8.04-7.97 (m, 2H), 7.32 (d, J = 7.9 Hz, 2H), 7.10 (d, J = 3.4 Hz, 1H), 6.22 (d, J = 3.4 Hz, 1H), 2.45 (s, 3H), 2.43 (s, 3H). |
| 1085 | 2-(4,5- dimethylthiazol- 2-yl)-5-(p-tolyl)- 1,3,4-thiadiazole | 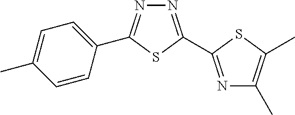 | 288.1 | ¹H NMR (500 MHz, CDCl₃) δ 7.93-7.78 (m, 2H), 7.29 (d, J = 7.8 Hz, 2H), 2.44 (s, 3H), 2.41 (d, J = 5.1 Hz, 6H). |
| 1086 | 2-(3-methyl-1H- pyrazol-4-yl)-5- (p-tolyl)-1,3,4- thiadiazole | 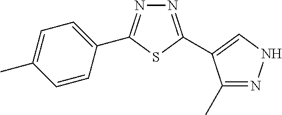 [SMILES: Cc1ccc(-c2nnc(-c3c[nH]nc3C)s2)cc1] | 257.1 | |
| 1087 | 2-(quinoxalin-2- yl)-5-(p-tolyl)- 1,3,4-thiadiazole | 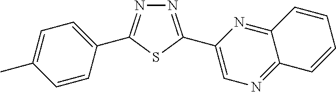 [SMILES: Cc1ccc(-c2nnc(-c3cnc4ccccc4n3)s2)cc1] | 305.1 | ¹H NMR (500 MHz, CDCl₃). 8 8.41-8.33 (m, 2H), 8.32- 8.26 (m, 1H), 8.19-8.16 (m, 2H), 7.90 (dd, J = 8.1, 1.4 Hz, 1H), 7.82 (ddd, J = 8.4, 6.9, 1.5 Hz, 1H), 7.65 (ddd, J = 8.1, 6.8, 1.2 Hz, 1H), 7.36 (d, J = 7.9 Hz, 1H), 2.46 (s, 3H) |
| 1088 | 4-amino-N-(5-(2- chlorophenyl)- 1,3,4-thiadiazol- 2-yl)benzamide | 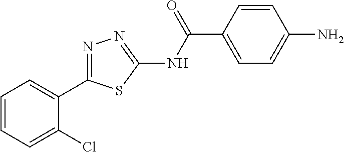 [SMILES: Nc1ccc(C(=O)Nc2nnc(-c3ccccc3Cl)s2)cc1] | 331.1 | |

### Example 2—Biological Activity of the ERR Modulating Compounds

HEK293 cells were maintained in Dulbecco's modified Eagles medium (DMEM) supplemented with 10% fetal bovine serum at 37° C. under 500 CO₂. Cells were plated in 96-well plates at a density of 2.5×10⁴ cells/well and transiently transfected using Lipofectamine 2000 (Invitrogen) according to manufacturer's instructions. Cells were transfected with ERRE reporter construct and pcDNA3.1 ERRα or ERRγ. Twenty-four-hours post-transfection, the cells were treated with vehicle or compound (various concentrations) for 24 hours (4 wells per dose). Luciferase activity was measured using the One-Glo Tox luciferase reporter assay system (Promega) and EC₅₀s were calculated using GraphPad Prism software.

TABLE 4

| | | EC₅₀ (ERRα) | EC₅₀ |
|---|---|---|---|
| Entry | Structure | μM | (ERRγ) μM |
| 1 | 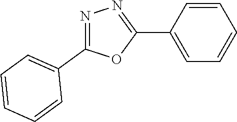 [SMILES: c1ccc(-c2nnc(-c3ccccc3)o2)cc1] | * | * |
| 2 | 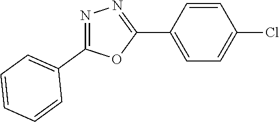 [SMILES: Clc1ccc(-c2nnc(-c3ccccc3)o2)cc1] | * | * |
| 3 | 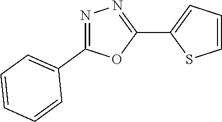 [SMILES: c1ccc(-c2nnc(-c3cccs3)o2)cc1] | * | * |
| 4 | 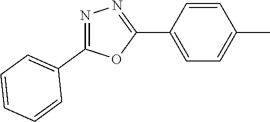 [SMILES: Cc1ccc(-c2nnc(-c3ccccc3)o2)cc1] | * | * |
| 5 | 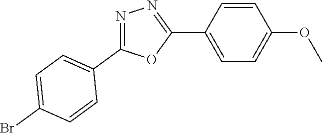 [SMILES: COc1ccc(-c2nnc(-c3ccc(Br)cc3)o2)cc1] | * | * |
| 6 | 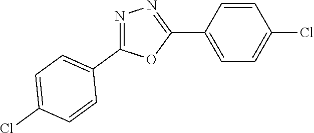 [SMILES: Clc1ccc(-c2nnc(-c3ccc(Cl)cc3)o2)cc1] | * | * |
| 7 | 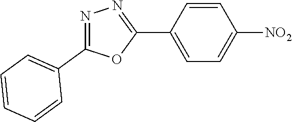 [SMILES: O=[N+]([O-])c1ccc(-c2nnc(-c3ccccc3)o2)cc1] | * | * |
| 8 | 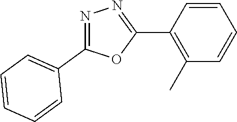 [SMILES: Cc1ccccc1-c1nnc(-c2ccccc2)o1] | * | * |
| 9 | 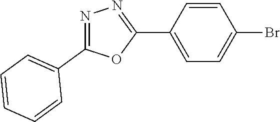 [SMILES: Brc1ccc(-c2nnc(-c3ccccc3)o2)cc1] | * | * |
| 10 | 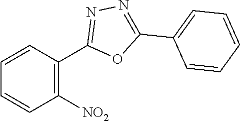 [SMILES: O=[N+]([O-])c1ccccc1-c1nnc(-c2ccccc2)o1] | ** | ** |
| 11 | 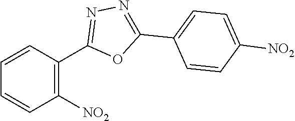 [SMILES: O=[N+]([O-])c1ccc(-c2nnc(-c3ccccc3[N+](=O)[O-])o2)cc1] | ** | ** |
| 12 | 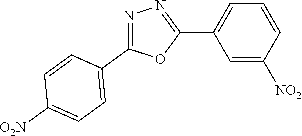 [SMILES: O=[N+]([O-])c1ccc(-c2nnc(-c3cccc([N+](=O)[O-])c3)o2)cc1] | ** | ** |
| 13 | 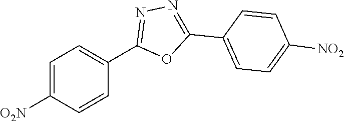 [SMILES: O=[N+]([O-])c1ccc(-c2nnc(-c3ccc([N+](=O)[O-])cc3)o2)cc1] | ** | ** |
| 14 | 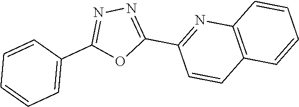 [SMILES: c1ccc(-c2nnc(-c3ccc4ccccc4n3)o2)cc1] | *** | *** |
| 15 | 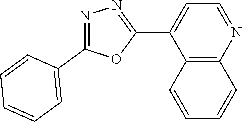 [SMILES: c1ccc(-c2nnc(-c3ccnc4ccccc34)o2)cc1] | i.a. | i.a. |
| 16 | 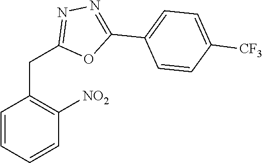 [SMILES: O=[N+]([O-])c1ccccc1Cc1nnc(-c2ccc(C(F)(F)F)cc2)o1] | * | * |
| 17 | 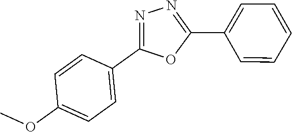 [SMILES: COc1ccc(-c2nnc(-c3ccccc3)o2)cc1] | * | * |
| 18 | 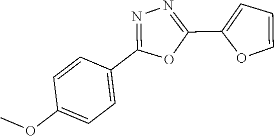 [SMILES: COc1ccc(-c2nnc(-c3ccco3)o2)cc1] | * | * |
| 19 | 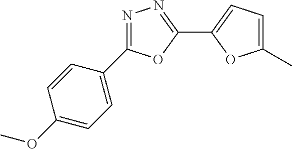 | * | * |
| 20 | 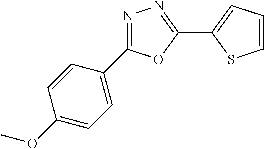 [SMILES: COc1ccc(-c2nnc(-c3cccs3)o2)cc1] | * | * |
| 21 | 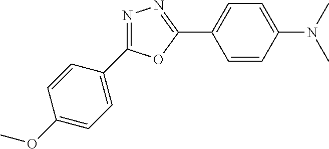 | ** | ** |
| 22 | 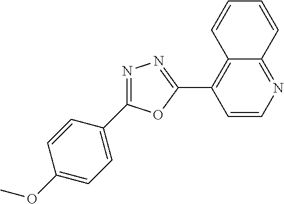 [SMILES: COc1ccc(-c2nnc(-c3ccnc4ccccc34)o2)cc1] | * | * |
| 23 | 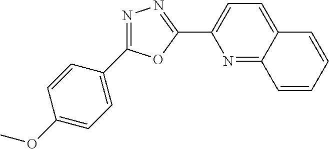 [SMILES: COc1ccc(-c2nnc(-c3ccc4ccccc4n3)o2)cc1] | * | * |
| 24 | 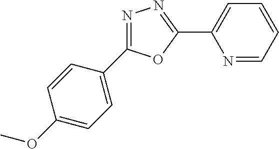 [SMILES: COc1ccc(-c2nnc(-c3ccccn3)o2)cc1] | i.a. | i.a. |
| 25 | 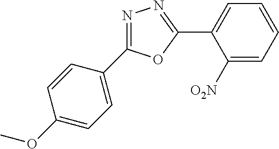 [SMILES: COc1ccc(-c2nnc(-c3ccccc3[N+](=O)[O-])o2)cc1] | * | * |
| 26 | 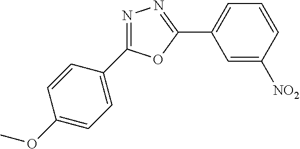 [SMILES: COc1ccc(-c2nnc(-c3cccc([N+](=O)[O-])c3)o2)cc1] | ** | * |
| 27 | 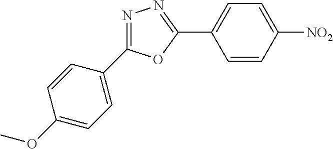 [SMILES: COc1ccc(-c2nnc(-c3ccc([N+](=O)[O-])cc3)o2)cc1] | N.T. | N.T. |
| 28 | 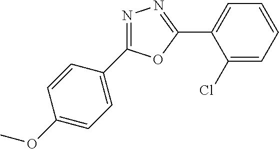 [SMILES: COc1ccc(-c2nnc(-c3ccccc3Cl)o2)cc1] | * | * |
| 29 | 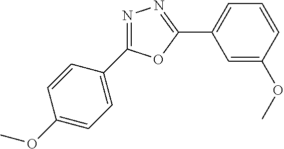 [SMILES: COc1ccc(-c2nnc(-c3cccc(OC)c3)o2)cc1] | * | * |
| 30 | 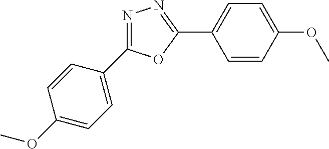 | * | * |
| 31 | 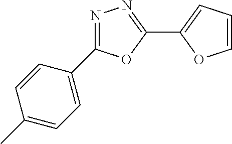 [SMILES: Cc1ccc(-c2nnc(-c3ccco3)o2)cc1] | * | * |
| 32 | 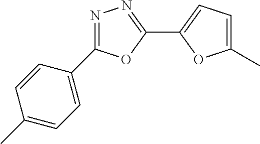 | * | * |
| 33 | 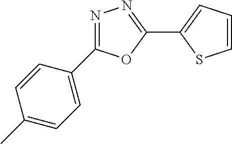 [SMILES: Cc1ccc(-c2nnc(-c3cccs3)o2)cc1] | * | * |
| 34 | 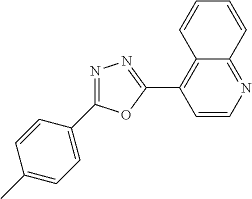 [SMILES: Cc1ccc(-c2nnc(-c3ccnc4ccccc34)o2)cc1] | * | * |
| 35 | 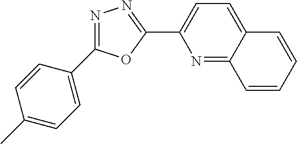 [SMILES: Cc1ccc(-c2nnc(-c3ccc4ccccc4n3)o2)cc1] | N.T. | N.T. |
| 36 | 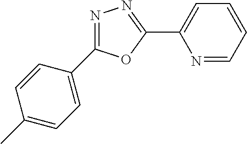 [SMILES: Cc1ccc(-c2nnc(-c3ccccn3)o2)cc1] | * | * |
| 37 | 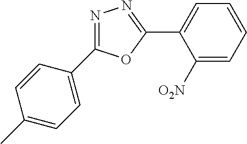 [SMILES: Cc1ccc(-c2nnc(-c3ccccc3[N+](=O)[O-])o2)cc1] | * | * |
| 38 | 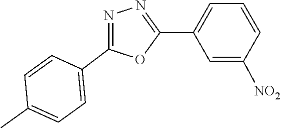 [SMILES: Cc1ccc(-c2nnc(-c3cccc([N+](=O)[O-])c3)o2)cc1] | * | * |
| 39 | 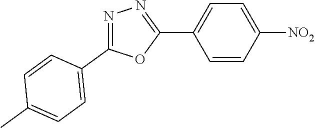 [SMILES: Cc1ccc(-c2nnc(-c3ccc([N+](=O)[O-])cc3)o2)cc1] | * | * |
| 40 | 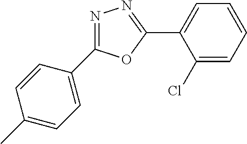 [SMILES: Cc1ccc(-c2nnc(-c3ccccc3Cl)o2)cc1] | * | * |
| 41 | 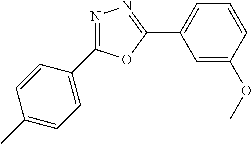 [SMILES: COc1cccc(-c2nnc(-c3ccc(C)cc3)o2)c1] | * | * |
| 42 | 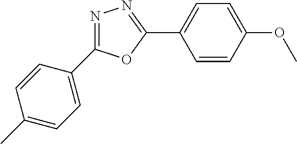 | * | * |
| 43 | 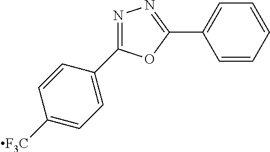 | ** | ** |
| 44 | 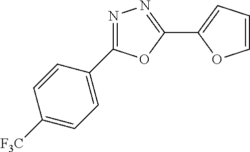 [SMILES: FC(F)(F)c1ccc(-c2nnc(-c3ccco3)o2)cc1] | ** | ** |
| 45 | 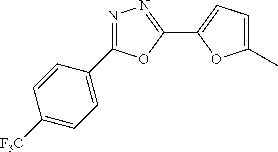 [SMILES: Cc1ccc(-c2nnc(-c3ccc(C(F)(F)F)cc3)o2)o1] | ** | *** |
| 46 | 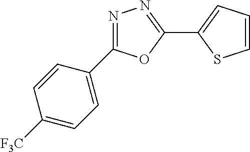 [SMILES: FC(F)(F)c1ccc(-c2nnc(-c3cccs3)o2)cc1] | ** | ** |
| 47 | 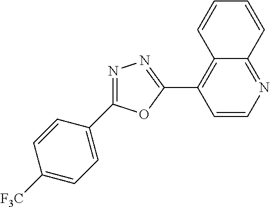 [SMILES: FC(F)(F)c1ccc(-c2nnc(-c3ccnc4ccccc34)o2)cc1] | *** | *** |
| 48 | 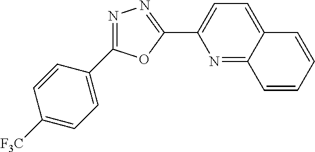 [SMILES: FC(F)(F)c1ccc(-c2nnc(-c3ccc4ccccc4n3)o2)cc1] | i.a. | i.a. |
| 49 | 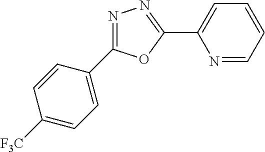 [SMILES: FC(F)(F)c1ccc(-c2nnc(-c3ccccn3)o2)cc1] | * | * |
| 50 | 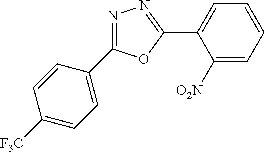 [SMILES: O=[N+]([O-])c1ccccc1-c1nnc(-c2ccc(C(F)(F)F)cc2)o1] | ** | ** |
| 51 | 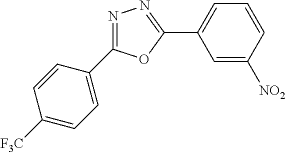 [SMILES: O=[N+]([O-])c1cccc(-c2nnc(-c3ccc(C(F)(F)F)cc3)o2)c1] | i.a. | i.a. |
| 52 | 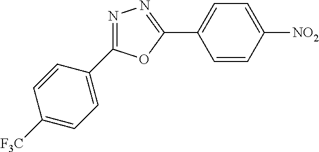 [SMILES: O=[N+]([O-])c1ccc(-c2nnc(-c3ccc(C(F)(F)F)cc3)o2)cc1] | *** | *** |
| 53 | 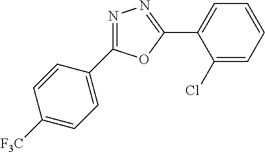 [SMILES: FC(F)(F)c1ccc(-c2nnc(-c3ccccc3Cl)o2)cc1] | * | ** |
| 54 | 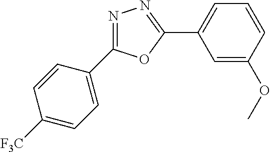 [SMILES: COc1cccc(-c2nnc(-c3ccc(C(F)(F)F)cc3)o2)c1] | *** | *** |
| 55 | 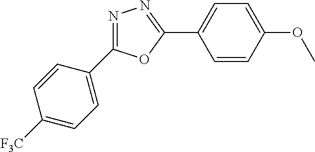 [SMILES: COc1ccc(-c2nnc(-c3ccc(C(F)(F)F)cc3)o2)cc1] | i.a. | i.a. |
| 56 | 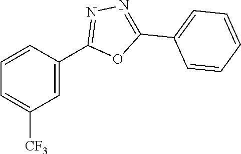 [SMILES: FC(F)(F)c1cccc(-c2nnc(-c3ccccc3)o2)c1] | * | * |
| 57 | 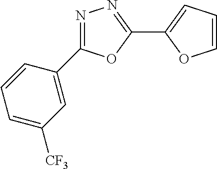 [SMILES: FC(F)(F)c1cccc(-c2nnc(-c3ccco3)o2)c1] | * | ** |
| 58 | 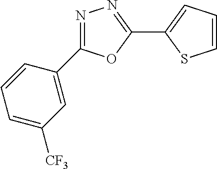 [SMILES: FC(F)(F)c1cccc(-c2nnc(-c3cccs3)o2)c1] | * | * |
| 59 | 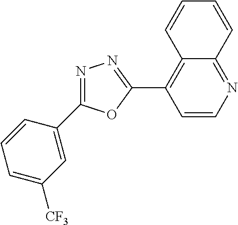 [SMILES: FC(F)(F)c1cccc(-c2nnc(-c3ccnc4ccccc34)o2)c1] | * | ** |
| 60 | 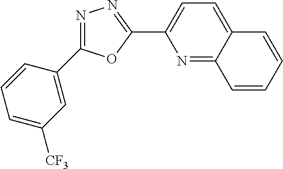 [SMILES: FC(F)(F)c1cccc(-c2nnc(-c3ccc4ccccc4n3)o2)c1] | ** | ** |
| 61 | 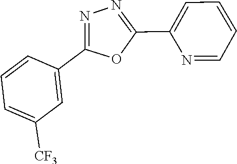 [SMILES: FC(F)(F)c1cccc(-c2nnc(-c3ccccn3)o2)c1] | * | * |
| 62 | 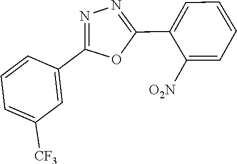 [SMILES: O=[N+]([O-])c1ccccc1-c1nnc(-c2cccc(C(F)(F)F)c2)o1] | ** | ** |
| 63 | 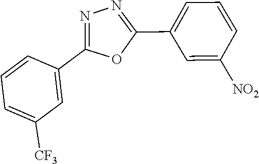 [SMILES: O=[N+]([O-])c1cccc(-c2nnc(-c3cccc(C(F)(F)F)c3)o2)c1] | ** | *** |
| 64 | 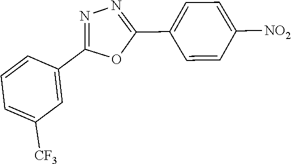 [SMILES: O=[N+]([O-])c1ccc(-c2nnc(-c3cccc(C(F)(F)F)c3)o2)cc1] | N.T. | *** |
| 65 | 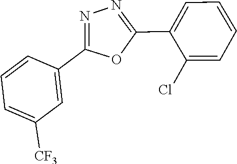 [SMILES: FC(F)(F)c1cccc(-c2nnc(-c3ccccc3Cl)o2)c1] | ** | *** |
| 66 | 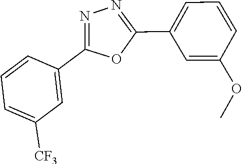 [SMILES: COc1cccc(-c2nnc(-c3cccc(C(F)(F)F)c3)o2)c1] | ** | ** |
| 67 | 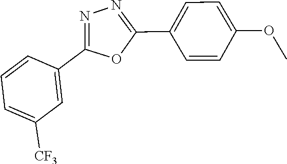 [SMILES: COc1ccc(-c2nnc(-c3cccc(C(F)(F)F)c3)o2)cc1] | ** | *** |
| 68 | 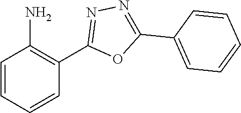 [SMILES: Nc1ccccc1-c1nnc(-c2ccccc2)o1] | ** | ** |
| 69 | 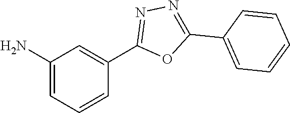 [SMILES: Nc1cccc(-c2nnc(-c3ccccc3)o2)c1] | * | * |
| 70 | 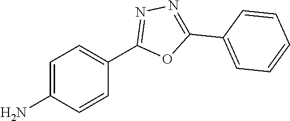 [SMILES: Nc1ccc(-c2nnc(-c3ccccc3)o2)cc1] | * | * |
| 71 | 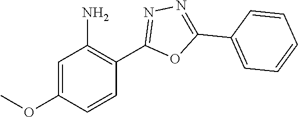 [SMILES: COc1ccc(-c2nnc(-c3ccccc3)o2)c(N)c1] | * | * |
| 72 | 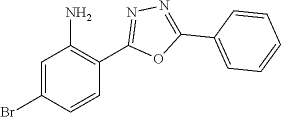 [SMILES: Nc1cc(Br)ccc1-c1nnc(-c2ccccc2)o1] | * | ** |
| 73 | 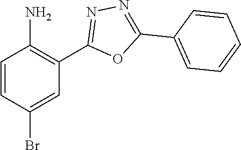 [SMILES: Nc1ccc(Br)cc1-c1nnc(-c2ccccc2)o1] | i.a. | i.a. |
| 74 | 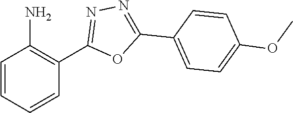 [SMILES: COc1ccc(-c2nnc(-c3ccccc3N)o2)cc1] | * | * |
| 75 | 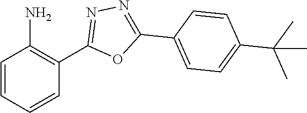 [SMILES: CC(C)(C)c1ccc(-c2nnc(-c3ccccc3N)o2)cc1] | * | * |
| 76 | 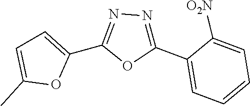 [SMILES: Cc1ccc(-c2nnc(-c3ccccc3[N+](=O)[O-])o2)o1] | ** | ** |
| 77 | 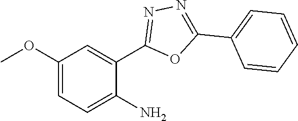 [SMILES: COc1ccc(N)c(-c2nnc(-c3ccccc3)o2)c1] | N.T. | N.T. |
| 78 | 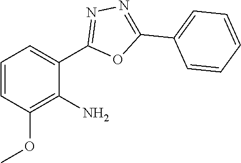 [SMILES: COc1cccc(-c2nnc(-c3ccccc3)o2)c1N] | N.T. | N.T. |
| 79 | 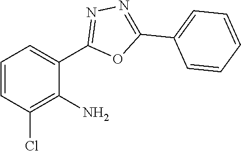 [SMILES: Nc1c(Cl)cccc1-c1nnc(-c2ccccc2)o1] | N.T. | N.T. |
| 80 | 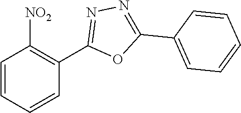 [SMILES: O=[N+]([O-])c1ccccc1-c1nnc(-c2ccccc2)o1] | ** | ** |
| 81 | 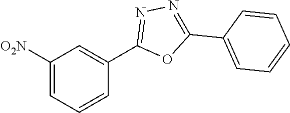 [SMILES: O=[N+]([O-])c1cccc(-c2nnc(-c3ccccc3)o2)c1] | ** | ** |
| 82 | 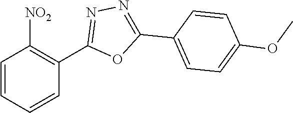 [SMILES: COc1ccc(-c2nnc(-c3ccccc3[N+](=O)[O-])o2)cc1] | * | * |
| 83 | 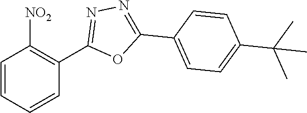 [SMILES: CC(C)(C)c1ccc(-c2nnc(-c3ccccc3[N+](=O)[O-])o2)cc1] | * | * |
| 84 | 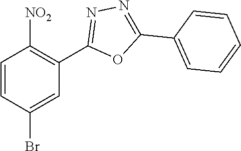 [SMILES: O=[N+]([O-])c1ccc(Br)cc1-c1nnc(-c2ccccc2)o1] | *** | *** |
| 85 | 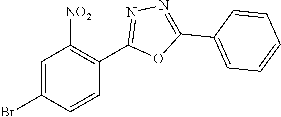 [SMILES: O=[N+]([O-])c1cc(Br)ccc1-c1nnc(-c2ccccc2)o1] | N.T. | N.T. |
| 86 | 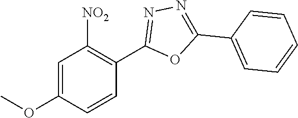 [SMILES: COc1ccc(-c2nnc(-c3ccccc3)o2)c([N+](=O)[O-])c1] | * | * |
| 87 | 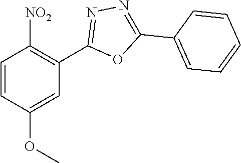 [SMILES: COc1ccc([N+](=O)[O-])c(-c2nnc(-c3ccccc3)o2)c1] | ** | *** |
| 88 | 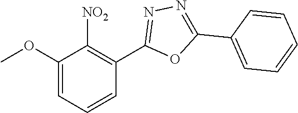 [SMILES: COc1cccc(-c2nnc(-c3ccccc3)o2)c1[N+](=O)[O-]] | * | * |
| 89 | 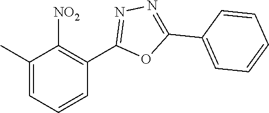 [SMILES: Cc1cccc(-c2nnc(-c3ccccc3)o2)c1[N+](=O)[O-]] | * | * |
| 90 | 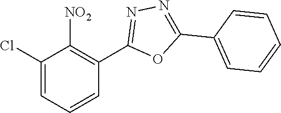 [SMILES: O=[N+]([O-])c1c(Cl)cccc1-c1nnc(-c2ccccc2)o1] | ** | ** |
| 91 | 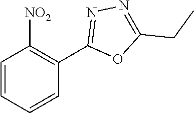 [SMILES: CCc1nnc(-c2ccccc2[N+](=O)[O-])o1] | N.T. | N.T. |
| 92 | 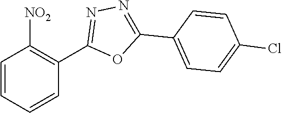 [SMILES: O=[N+]([O-])c1ccccc1-c1nnc(-c2ccc(Cl)cc2)o1] | *** | *** |
| 93 | 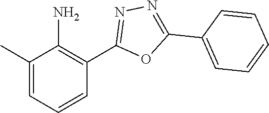 [SMILES: Cc1cccc(-c2nnc(-c3ccccc3)o2)c1N] | N.T. | N.T. |
| 94 | 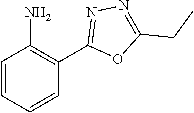 | N.T. | N.T. |
| 95 | 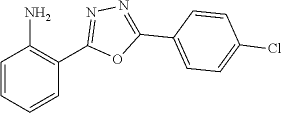 [SMILES: Nc1ccccc1-c1nnc(-c2ccc(Cl)cc2)o1] | N.T. | N.T. |
| 1001 | 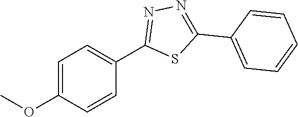 [SMILES: COc1ccc(-c2nnc(-c3ccccc3)s2)cc1] | * | * |
| 1002 | 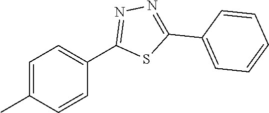 [SMILES: Cc1ccc(-c2nnc(-c3ccccc3)s2)cc1] | * | * |
| 1003 | 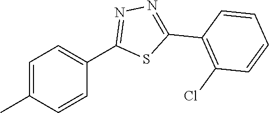 [SMILES: Cc1ccc(-c2nnc(-c3ccccc3Cl)s2)cc1] | * | * |
| 1004 | 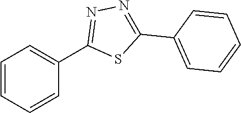 [SMILES: c1ccc(-c2nnc(-c3ccccc3)s2)cc1] | * | * |
| 1005 | 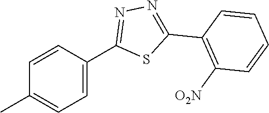 [SMILES: Cc1ccc(-c2nnc(-c3ccccc3[N+](=O)[O-])s2)cc1] | * | * |
| 1006 | 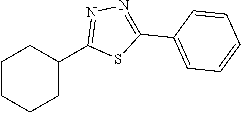 [SMILES: c1ccc(-c2nnc(C3CCCCC3)s2)cc1] | * | * |
| 1007 | 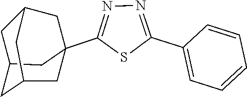 [SMILES: c1ccc(-c2nnc(C34CC5CC(CC(C5)C3)C4)s2)cc1] | * | * |
| 1008 | 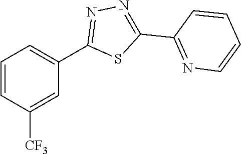 [SMILES: FC(F)(F)c1cccc(-c2nnc(-c3ccccn3)s2)c1] | * | * |
| 1009 | 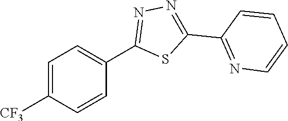 [SMILES: FC(F)(F)c1ccc(-c2nnc(-c3ccccn3)s2)cc1] | * | * |
| 1010 | 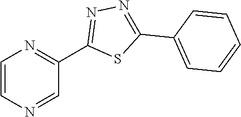 [SMILES: c1ccc(-c2nnc(-c3cnccn3)s2)cc1] | * | * |
| 1011 | 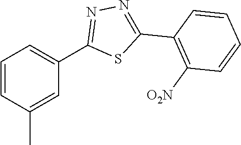 [SMILES: Cc1cccc(-c2nnc(-c3ccccc3[N+](=O)[O-])s2)c1] | N.T. | N.T. |
| 1012 | 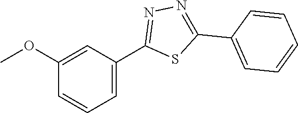 [SMILES: COc1cccc(-c2nnc(-c3ccccc3)s2)c1] | * | * |
| 1013 | 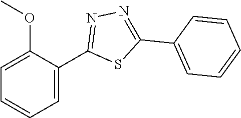 [SMILES: COc1ccccc1-c1nnc(-c2ccccc2)s1] | * | * |
| 1014 | 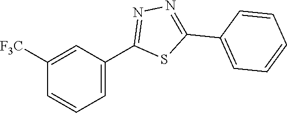 [SMILES: FC(F)(F)c1cccc(-c2nnc(-c3ccccc3)s2)c1] | ** | ** |
| 1015 | 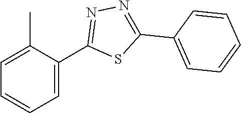 [SMILES: Cc1ccccc1-c1nnc(-c2ccccc2)s1] | * | * |
| 1016 | 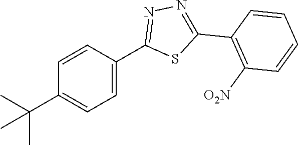 [SMILES: CC(C)(C)c1ccc(-c2nnc(-c3ccccc3[N+](=O)[O-])s2)cc1] | * | * |
| 1017 | 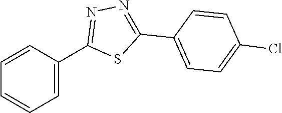 [SMILES: Clc1ccc(-c2nnc(-c3ccccc3)s2)cc1] | * | * |
| 1018 | 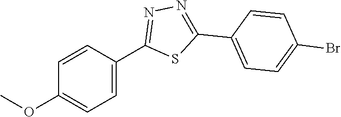 [SMILES: COc1ccc(-c2nnc(-c3ccc(Br)cc3)s2)cc1] | * | ** |
| 1019 | 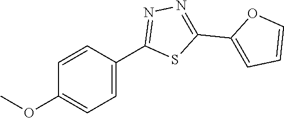 [SMILES: COc1ccc(-c2nnc(-c3ccco3)s2)cc1] | * | * |
| 1020 | 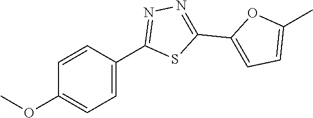 | * | * |
| 1021 | 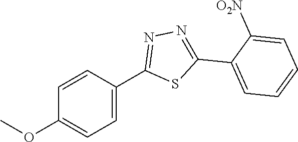 [SMILES: COc1ccc(-c2nnc(-c3ccccc3[N+](=O)[O-])s2)cc1] | * | * |
| 1022 | 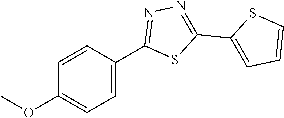 [SMILES: COc1ccc(-c2nnc(-c3cccs3)s2)cc1] | * | * |
| 1023 | 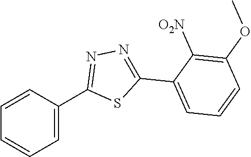 [SMILES: COc1cccc(-c2nnc(-c3ccccc3)s2)c1[N+](=O)[O-]] | * | * |
| 1024 | 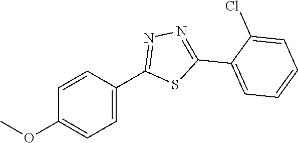 [SMILES: COc1ccc(-c2nnc(-c3ccccc3Cl)s2)cc1] | * | * |
| 1025 | 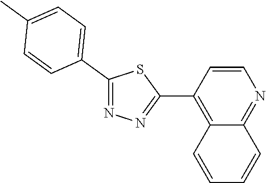 [SMILES: Cc1ccc(-c2nnc(-c3ccnc4ccccc34)s2)cc1] | * | * |
| 1026 | 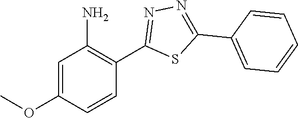 [SMILES: COc1ccc(-c2nnc(-c3ccccc3)s2)c(N)c1] | *** | *** |
| 1027 | 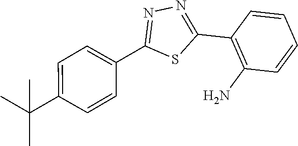 [SMILES: CC(C)(C)c1ccc(-c2nnc(-c3ccccc3N)s2)cc1] | * | * |
| 1028 | 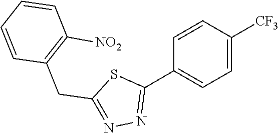 [SMILES: O=[N+]([O-])c1ccccc1Cc1nnc(-c2ccc(C(F)(F)F)cc2)s1] | ** | ** |
| 1029 | 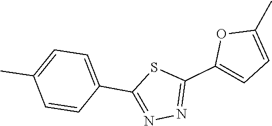 | ** | * |
| 1030 | 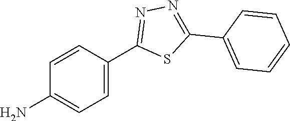 [SMILES: Nc1ccc(-c2nnc(-c3ccccc3)s2)cc1] | * | * |
| 1031 | 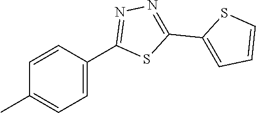 [SMILES: Cc1ccc(-c2nnc(-c3cccs3)s2)cc1] | * | * |
| 1032 | 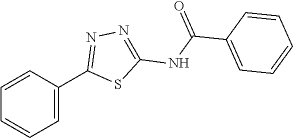 [SMILES: O=C(Nc1nnc(-c2ccccc2)s1)c1ccccc1] | i.a. | i.a. |
| 1033 | 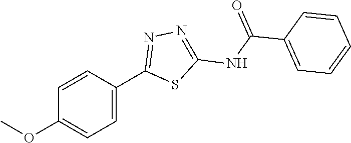 [SMILES: COc1ccc(-c2nnc(NC(=O)c3ccccc3)s2)cc1] | i.a. | i.a. |
| 1034 | 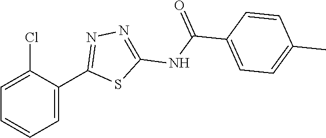 [SMILES: Cc1ccc(C(=O)Nc2nnc(-c3ccccc3Cl)s2)cc1] | i.a. | i.a. |
| 1035 | 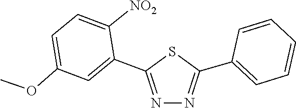 [SMILES: COc1ccc([N+](=O)[O-])c(-c2nnc(-c3ccccc3)s2)c1] | ** | ** |
| 1036 | 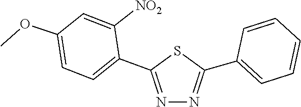 [SMILES: COc1ccc(-c2nnc(-c3ccccc3)s2)c([N+](=O)[O-])c1] | * | * |
| 1037 | 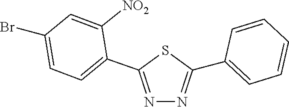 [SMILES: O=[N+]([O-])c1cc(Br)ccc1-c1nnc(-c2ccccc2)s1] | ** | * |
| 1038 | 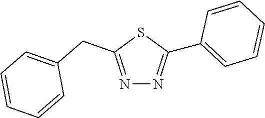 [SMILES: c1ccc(Cc2nnc(-c3ccccc3)s2)cc1] | ** | ** |
| 1039 | 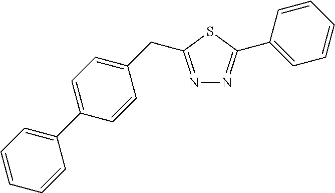 [SMILES: c1ccc(-c2ccc(Cc3nnc(-c4ccccc4)s3)cc2)cc1] | *** | *** |
| 1040 | 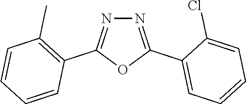 [SMILES: Cc1ccccc1-c1nnc(-c2ccccc2Cl)o1] | ** | * |
| 1041 | 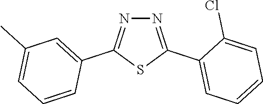 [SMILES: Cc1cccc(-c2nnc(-c3ccccc3Cl)s2)c1] | *** | * |
| 1042 | 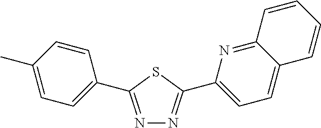 [SMILES: Cc1ccc(-c2nnc(-c3ccc4ccccc4n3)s2)cc1] | *** | * |
| 1043 | 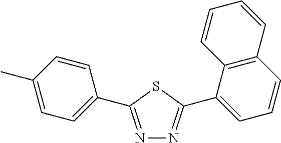 [SMILES: Cc1ccc(-c2nnc(-c3cccc4ccccc34)s2)cc1] | * | * |
| 1044 | 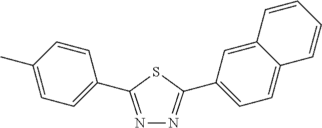 [SMILES: Cc1ccc(-c2nnc(-c3ccc4ccccc4c3)s2)cc1] | *** | * |
| 1045 | 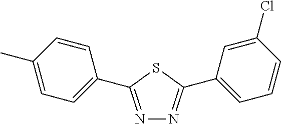 [SMILES: Cc1ccc(-c2nnc(-c3cccc(Cl)c3)s2)cc1] | * | * |
| 1046 | 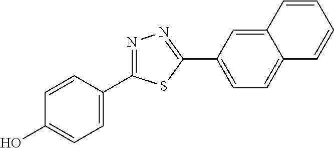 [SMILES: Oc1ccc(-c2nnc(-c3ccc4ccccc4c3)s2)cc1] | * | * |
| 1047 | 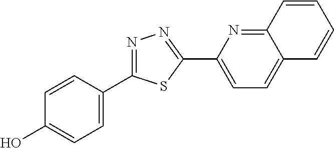 [SMILES: Oc1ccc(-c2nnc(-c3ccc4ccccc4n3)s2)cc1] | i.a. | i.a. |
| 1048 | 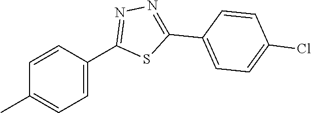 [SMILES: Cc1ccc(-c2nnc(-c3ccc(Cl)cc3)s2)cc1] | * | * |
| 1049 | 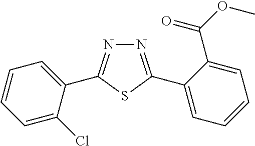 [SMILES: COC(=O)c1ccccc1-c1nnc(-c2ccccc2Cl)s1] | N.T. | N.T. |
| 1050 | 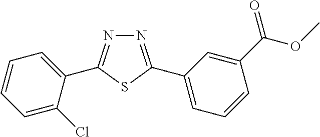 [SMILES: COC(=O)c1cccc(-c2nnc(-c3ccccc3Cl)s2)c1] | * | * |
| 1051 | 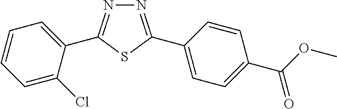 [SMILES: COC(=O)c1ccc(-c2nnc(-c3ccccc3Cl)s2)cc1] | * | * |
| 1052 | 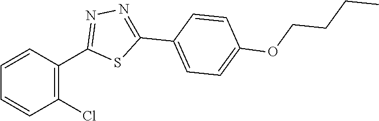 [SMILES: CCCCOc1ccc(-c2nnc(-c3ccccc3Cl)s2)cc1] | * | * |
| 1053 | 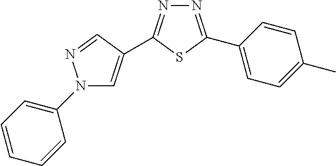 [SMILES: Cc1ccc(-c2nnc(-c3cnn(-c4ccccc4)c3)s2)cc1] | *** | * |
| 1054 | 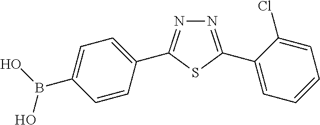 [SMILES: OB(O)c1ccc(-c2nnc(-c3ccccc3Cl)s2)cc1] | N.T. | N.T. |
| 1055 | 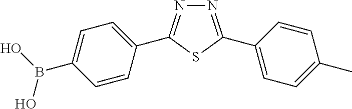 [SMILES: Cc1ccc(-c2nnc(-c3ccc(B(O)O)cc3)s2)cc1] | * | * |
| 1056 | 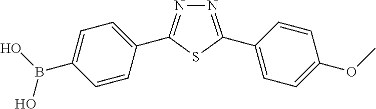 [SMILES: COc1ccc(-c2nnc(-c3ccc(B(O)O)cc3)s2)cc1] | N.T. | N.T. |
| 1057 | 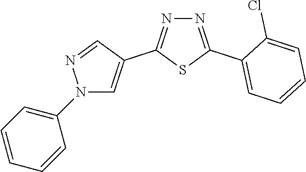 [SMILES: Clc1ccccc1-c1nnc(-c2cnn(-c3ccccc3)c2)s1] | N.T. | N.T. |
| 1058 | 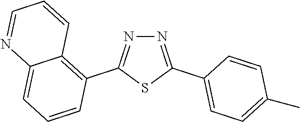 [SMILES: Cc1ccc(-c2nnc(-c3cccc4ncccc34)s2)cc1] | * | * |
| 1059 | 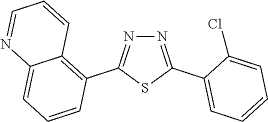 [SMILES: Clc1ccccc1-c1nnc(-c2cccc3ncccc23)s1] | * | * |
| 1060 | 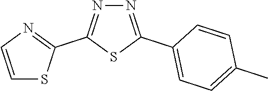 [SMILES: Cc1ccc(-c2nnc(-c3nccs3)s2)cc1] | * | * |
| 1061 | 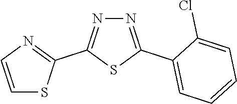 [SMILES: Clc1ccccc1-c1nnc(-c2nccs2)s1] | *** | *** |
| 1062 | 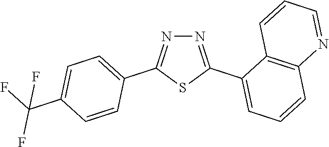 [SMILES: FC(F)(F)c1ccc(-c2nnc(-c3cccc4ncccc34)s2)cc1] | * | * |
| 1063 | 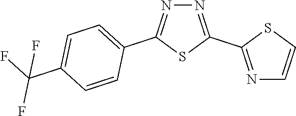 [SMILES: FC(F)(F)c1ccc(-c2nnc(-c3nccs3)s2)cc1] | ** | ** |
| 1064 | 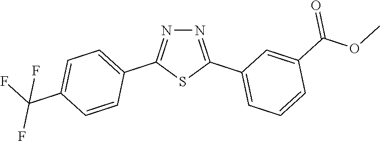 [SMILES: COC(=O)c1cccc(-c2nnc(-c3ccc(C(F)(F)F)cc3)s2)c1] | *** | ** |
| 1065 | 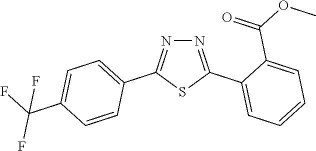 [SMILES: COC(=O)c1ccccc1-c1nnc(-c2ccc(C(F)(F)F)cc2)s1] | N.T. | N.T. |
| 1066 | 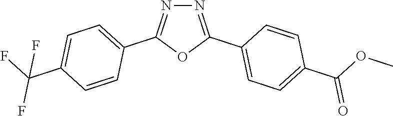 [SMILES: COC(=O)c1ccc(-c2nnc(-c3ccc(C(F)(F)F)cc3)o2)cc1] | ** | ** |
| 1067 | 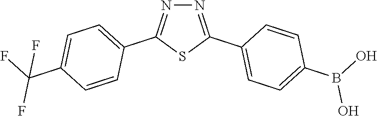 [SMILES: OB(O)c1ccc(-c2nnc(-c3ccc(C(F)(F)F)cc3)s2)cc1] | N.T. | N.T. |
| 1068 | 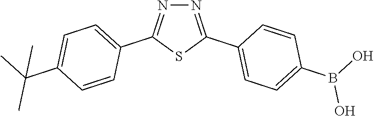 [SMILES: CC(C)(C)c1ccc(-c2nnc(-c3ccc(B(O)O)cc3)s2)cc1] | N.T. | N.T. |
| 1069 | 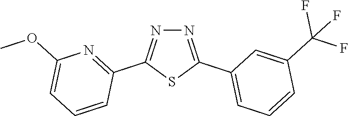 [SMILES: COc1cccc(-c2nnc(-c3cccc(C(F)(F)F)c3)s2)n1] | | |
| 1070 | 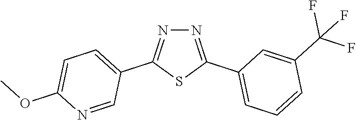 [SMILES: COc1ccc(-c2nnc(-c3cccc(C(F)(F)F)c3)s2)cn1] | N.T. | N.T. |
| 1071 | 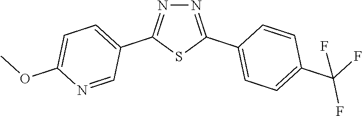 [SMILES: COc1ccc(-c2nnc(-c3ccc(C(F)(F)F)cc3)s2)cn1] | * | ** |
| 1072 | 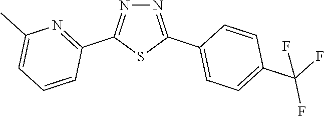 [SMILES: Cc1cccc(-c2nnc(-c3ccc(C(F)(F)F)cc3)s2)n1] | N.T. | N.T. |
| 1073 | 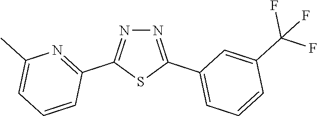 [SMILES: Cc1cccc(-c2nnc(-c3cccc(C(F)(F)F)c3)s2)n1] | *** | ** |
| 1074 | 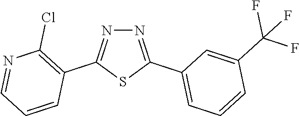 [SMILES: FC(F)(F)c1cccc(-c2nnc(-c3cccnc3Cl)s2)c1] | ** | ** |
| 1075 | 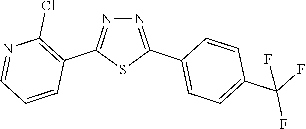 [SMILES: FC(F)(F)c1ccc(-c2nnc(-c3cccnc3Cl)s2)cc1] | N.T. | N.T. |
| 1076 | 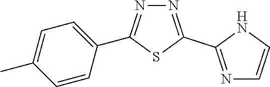 [SMILES: Cc1ccc(-c2nnc(-c3ncc[nH]3)s2)cc1] | | |
| 1077 | 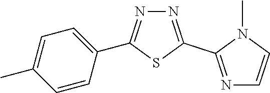 [SMILES: Cc1ccc(-c2nnc(-c3nccn3C)s2)cc1] | N.T. | N.T. |
| 1078 | 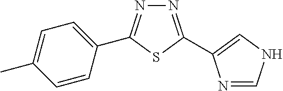 [SMILES: Cc1ccc(-c2nnc(-c3c[nH]cn3)s2)cc1] | N.T. | N.T. |
| 1079 | 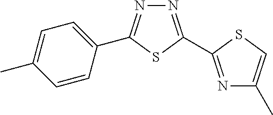 | | |
| 1080 | 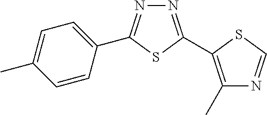 [SMILES: Cc1ccc(-c2nnc(-c3scnc3C)s2)cc1] | * | * |
| 1081 | 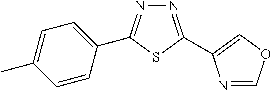 [SMILES: Cc1ccc(-c2nnc(-c3cocn3)s2)cc1] | N.T. | N.T. |
| 1082 | 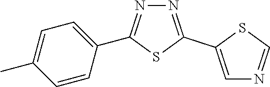 [SMILES: Cc1ccc(-c2nnc(-c3cncs3)s2)cc1] | * | * |
| 1083 | 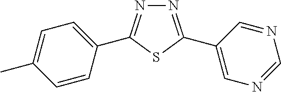 [SMILES: Cc1ccc(-c2nnc(-c3cncnc3)s2)cc1] | N.T. | N.T. |
| 1084 | 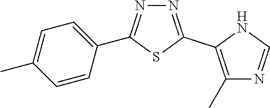 [SMILES: Cc1ccc(-c2nnc(-c3[nH]cnc3C)s2)cc1] | | |
| 1085 | 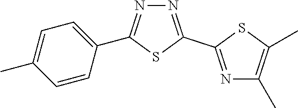 | | |
| 1086 | 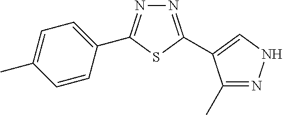 [SMILES: Cc1ccc(-c2nnc(-c3c[nH]nc3C)s2)cc1] | N.T. | N.T. |
| 1087 | 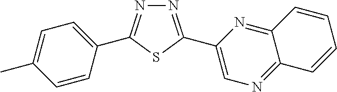 [SMILES: Cc1ccc(-c2nnc(-c3cnc4ccccc4n3)s2)cc1] | | |

n.t. (not tested)
i.a. (inactive)
* <1 μM
** 1-5 μM
*** >5 μM

While the compositions and methods of this disclosure have been described in terms of embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed:

1. A method of modulating the activity of an estrogen related receptor comprising contacting the estrogen related receptor with a compound of the formula: 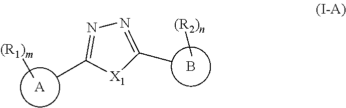
wherein:
X₁ is O or S;
A is C₁₋₆ alkyl-aryl or C₆ aryl;
B is C₁₋₆ alkyl, C₆₋₁₀ cycloalkyl, C₁₋₆ alkyl-C₆₋₁₀ aryl, SC(O)-aryl, C₆₋₁₀ aryl, 5-10 membered heteroaryl, or N(H)C(O)-aryl;
R₁ is H, halo, OH, N(R₃)₂, C₁₋₆ alkyl, C₁₋₆ haloalkyl, C₁₋₆ alkoxy, C(O)C₁₋₆ alkoxy, or B(OH)₂;
R₂ is H, halo, N(R₃)₂, N(R₄)₂, C₁₋₆ alkyl, C₁₋₆ haloalkyl, C₁₋₆ alkoxy, C(O)C₁₋₆ alkoxy, C(O)OH, N(H)C(O)—C₃₋₆ cycloalkyl, or aryl;
R₃ is H or O;
R₄ is O or C₁₋₆ alkyl;
m is 0, 1, or 2; and
n is 0, 1, or 2;
or a pharmaceutically acceptable salt thereof.

2. The method of claim 1, wherein the compound is further defined as: 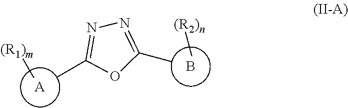
wherein:
A is C₁₋₆ alkyl-aryl or C₆ aryl;
B is C₁₋₆ alkyl, C₁₋₆ alkyl-C₆₋₁₀ aryl, SC(O)-aryl, C₆₋₁₀ aryl, 5-10 membered heteroaryl, or N(H)C(O)-aryl;
R₁ is H, halo, OH, N(R₃)₂, C₁₋₆ alkyl, C₁₋₆ haloalkyl, C₁₋₆ alkoxy, C(O)C₁₋₆ alkoxy, or B(OH)₂;
R₂ is H, halo, N(R₄)₂, C₁₋₆ alkyl, C₁₋₆ haloalkyl, C₁₋₆ alkoxy, C(O)C₁₋₆ alkoxy, C(O)OH, or N(H)C(O)—C₃₋₆ cycloalkyl;
R₃ is H or O;
R₄ is O or C₁₋₆ alkyl;
m is 0, 1, or 2; and
n is 0 or 1;
or a pharmaceutically acceptable salt thereof.

3. The method of claim 1, wherein the compound is further defined as: 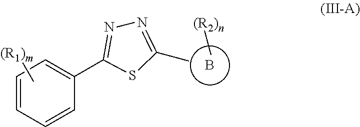
wherein:
B is C₁₋₆ alkyl-aryl, C₆₋₁₀ cycloalkyl, C₆₋₁₀ aryl, 5-10 membered heteroaryl, or N(H)C(O)-aryl;
R₁ is H, halo, OH, N(R₃)₂, C₁₋₆ alkyl, C₁₋₆ haloalkyl, C₁₋₆ alkoxy, or B(OH)₂;
R₂ is H, halo, N(R₃)₂, C₁₋₆ alkyl, C₁₋₆ haloalkyl, C₁₋₆ alkoxy, C(O)C₁₋₆ alkoxy, or aryl;
R₃ is H or O;
m is 0, 1, or 2; and
n is 0, 1, or 2;
or a pharmaceutically acceptable salt thereof.

4. The method of claim 1, wherein A or B is phenyl.

5. The method of claim 1, wherein B is 5-10 membered heteroaryl.

6. The method of claim 1, wherein R₁ is selected from the group consisting of C₁₋₆ alkyl, C₁₋₆ haloalkyl, halo, B(OH)₂, or C₁₋₆ alkoxy.

7. The method of claim 1, wherein R₁ is selected from the group consisting of B(OH)₂, CF₃, CH₃, and OCH₃.

8. The method of claim 1, wherein R₁ is B(OH)₂, Br, C(CH₃)₃, CF₃, CH₃, Cl, NH₂, OCH₃, OH, NO₂, or C(O)OCH₃.

9. The method of claim 1, wherein R₂ is selected from the group consisting of halo N(R₃)₂, C₁₋₆ alkyl, C₁₋₆ haloalkyl, and C₁₋₆ alkoxy.

10. The method of claim 1, wherein R₂ is C(CH₃)₃, C(O)OCH₃, C(O)OH, CF₃, CH₃, Cl, N(CH₃)₂, N(H)C(O)-cyclobutyl, N(H)C(O)-cyclohexyl, N(H)C(O)-cyclopropyl, NO₂, O(CH₂)₃CH₃, OCH₃, OH, Br, NH₂, or phenyl.

11. The method of claim 1, wherein R₃ is O or H.

12. The method of claim 1, wherein R₄ is O or C₁₋₆ alkyl.

13. The method of claim 1, wherein the disease or disorder is selected from the group consisting of diabetes, breast cancer, bone disease, bone resorption, heart failure, obesity, metabolic disease, muscle wasting and other diseases of muscle function, diseases of mitochongrial disfunction, kidney diseases and disfunction, and neurodegenerative diseases including AD, PD, and ALS.
